(19) United States
(12) Patent Application Publication
(10) Pub. No.: US 2022/0265870 A1
(43) Pub. Date: Aug. 25, 2022

(54) MULTIVALENT FIBROBLAST-TARGETED AGENTS AND METHODS OF USE

(71) Applicant: Purdue Research Foundation, West Lafayette, IN (US)

(72) Inventors: Philip Stewart Low et al., West Lafayette, IN (US); Madduri Srinivasarao, West Lafayette, IN (US); Ramesh Mukkamala, West Lafayette, IN (US)

(21) Appl. No.: 17/629,252

(22) PCT Filed: Jul. 22, 2020
(86) PCT No.: PCT/US2020/043141

§ 371 Date: Jan. 21, 2022

Related U.S. Application Data
(60) Provisional application No. 62/877,039, filed on Jul. 22, 2019.
(60) Provisional application No. 62/910,764, filed on Oct. 4, 2019.
(60) Provisional application No. 62/933,655, filed on Nov. 11, 2019.

(51) Int. Cl. A61K 51/04; A61K 47/54; A61K 49/00; A61P 35/00

(52) U.S. Cl. CPC A61K 51/0497; A61K 47/545; A61K 49/0032; A61P 35/00; A61K 49/0052; A61K 51/0446; A61K 49/0043

(57) ABSTRACT

Multivalent ligand-targeted active agents, such as detectable agents or therapeutic agents, for the imaging and treatment, respectively, of fibroblast activation protein (FAP)-positive cancer-associated fibroblasts (CAFs) and activated myofibroblasts in cancers and other fibrotic diseases.

### CROSS-REFERENCE TO RELATED APPLICATIONS

This application claims the benefit of U.S. Provisional Patent Application No. 62/877,039, filed on Jul. 22, 2019, U.S. Provisional Patent Application No. 62/910,764, filed on Oct. 4, 2019, and U.S. Provisional Patent Application No. 62/933,655, filed on Nov. 11, 2019, which are all hereby incorporated by reference in their entireties.

### TECHNICAL FIELD

The present application relates to multivalent compounds of ligands and active agents (e.g., therapeutic agents and imaging agents) that target fibroblasts, including cancer-associated fibroblasts (CAFs). Internalization and residence time of the multivalent compounds are enhanced in tumors and other diseased sites.

### BACKGROUND

The tumor microenvironment (TME), or the environment surrounding a tumor (e.g., surrounding blood vessels, immune cells, fibroblasts, extracellular matrix, etc.), can play a role in the development of cancers. One of the components (e.g., critical components) of the TME are cancer-associated fibroblasts (CAFs). Through the secretion of various cytokines, growth factors, and collagen, the CAFs can support the survival, growth and metastasis of tumors. In order to address the need for new approaches to treating tumors or CAF-related diseases, presented herein are multivalent compounds that target CAFs and/or targets disposed thereon.

CAFs can overexpress fibroblast activation protein (FAP) on their cell surfaces, which can be exploited to deliver drugs and imaging agents for the treatment and detection of cancer, respectively. FAP is a type II, membrane-bound serine protease that cleaves proline-amino acid peptide bonds. FAP-targeted drugs and imaging agents have recently been reported for cancer and other fibrotic diseases. Although FAP ligand-targeted drug and imaging agents are known, their usefulness is limited by their poor internalization and shorter residence time at the diseased site. There remains a need to develop FAP ligand-targeted drugs and imaging agents with increased internalization and longer residence time at the disease site.

### SUMMARY

Provided is a multivalent compound (or conjugate) having the structure (Q-L^{Q})ₘ-Y-L^{X}-X, wherein
each Q-L^{Q} is an arm of the multivalent compound;
m is the number of (Q-L^{Q}) arms in the multivalent compound and is an integer 2, 3, 4, 5, or 6;
Q is a ligand that binds to fibroblast activation protein (FAP) on a target cell;
L^{Q} is a spacer that (i) connects Q to Y and (ii) provides length for the arms of the multivalent compound to reach multiple adjacent FAPs on the target cell;
Y is a multipoint template to which the multiple arms of the multivalent compound connect;
L^{X} is a spacer that connects X to Y; and
X is an active agent.

A multivalent compound can have the structure:

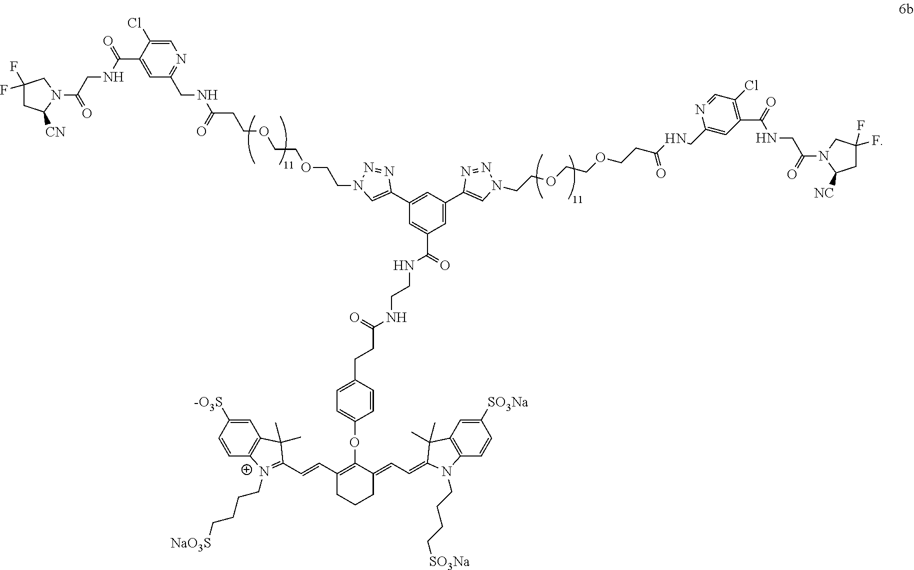

A multivalent compound can have the structure:

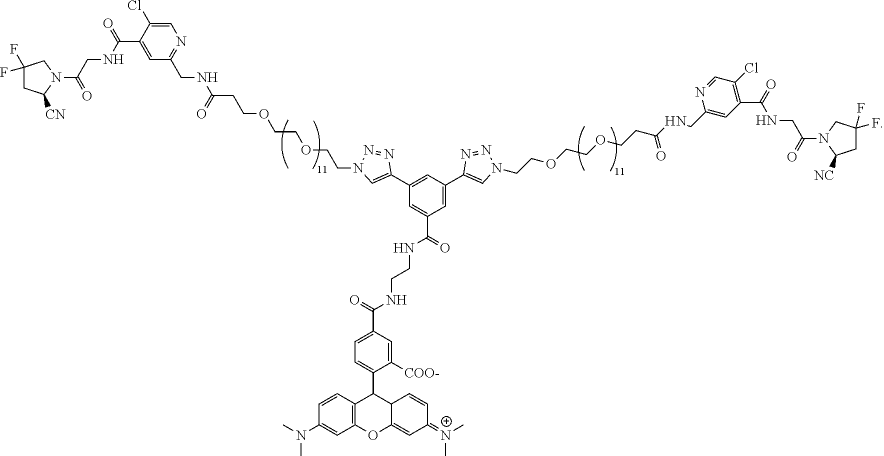

Also provided is a pharmaceutical composition comprising a multivalent compound and a pharmaceutically acceptable carrier.

A method of providing an active agent in proximity to a CAF or FAP-expressing cell is further provided. The method comprises administering a compound to a CAF or a FAP-expressing cell, and the compound is retained within or upon the CAF or FAP-expressing cell for at least 24 hours.

A method of providing an active agent in proximity to a CAF or FAP-expressing cell is still further provided. The method comprises administering a compound to a subject comprising or suspected of comprising a plurality of CAFs or FAP-expressing cells, wherein the compound is retained within the CAFs or FAP-expressing cells for at least 24 hours.

Also provided is a method of detecting a tumor or fibrotic tissue in a subject. The method comprises (i) administering a compound to a subject in need thereof, (ii) detecting the compound within the subject (e.g., optically or radiometrically), and (iii) identifying the tumor or fibrotic tissue in the subject based on the localization of the compound.

A method of treating a tumor or fibrotic tissue in a subject is also provided. The method comprises administering to the subject a therapeutically effective amount of a compound.

### BRIEF DESCRIPTION OF THE DRAWINGS

FIG. 1 is a dimeric space-filling model of fibroblast activation protein (FAP) based on a 2.6 Å resolution crystal structure (PDB code: 1z68) comprised of two identical peptide chains.

FIG. 2 is the depth of the active site of FAP, based on the crystal structure-derived model shown in FIG. 1.

FIG. 3 is an example synthesis of a (Q-L^{Q})₂ multipoint template for delivery of active agents (e.g., therapeutic or imaging agents).

FIG. 4A is an example synthesis of a tert-butyloxycarbonyl (Boc)-protected (Q-L^{Q})₂-Y-L^{X}-Boc multipoint template prior to functionalization with an active agent X.

FIG. 4B is the synthesis of rhodamine compounds, a rhodamine-based active agent, following Boc deprotection and rhodamine coupling.

FIG. 5 is an example synthesis of multivalent conjugate with a S0456-based dye as the active agent.

FIG. 6 shows a liquid chromatography-mass spectrometry (LC-MS) trace of multivalent conjugate 6b.

FIG. 7 shows a liquid chromatography-mass spectrometry (LC-MS) trace of a multivalent conjugate.

FIG. 8A-D show binding studies for (Q-L^{Q})₁-Y-L^{X}-X “mono-FAP” and (Q-L^{Q})₂-Y-L^{X}-X “dual-FAP” multivalent compounds using confocal microscopy. Readings taken at 1 h and 8 h show significant retention in cells for both mono-FAP and dual-FAP compounds after incubating at 37° C. for 1 h. After 8 h incubation, the dual-FAP compound remained clearly visible within cells, while the mono-FAP compound was greatly diminished in detectability under equivalent conditions.

FIG. 9A-C illustrate binding studies for mono-FAP and dual-FAP multivalent compounds using confocal microscopy after 24 h and 48 h incubation. The dual-FAP conjugate was retained in cells for up to 48 h.

FIG. 10A-B illustrate binding studies of dual-FAP multivalent compounds on non-FAP HT1080 cells at 12.5 and 25 nM concentrations, showing that binding of dual-FAP compounds is FAP-specific.

FIG. 11A-D show in vivo imaging of multivalent compound 6b on KB tumor-bearing mice at 18 h, 24 h, and 48 h following administration of multivalent compound 6b.

FIG. 12A-C show in vivo imaging of multivalent compound 6b on KB tumor-bearing mice at 72 h, 96 h, and 114 h following administration of multivalent conjugate 6b.

FIG. 13A-B illustrate the biodistribution of dual-FAP multivalent compound 6b at 114 h post-injection.

FIG. 14A-B show a competition experiment in mice between a detectable multivalent compound and a 100-fold excess of unlabeled multivalent compound. In the absence of excess unlabeled compound (targeted), uptake of the detectable compound was substantially greater than in the presence (competition) of excess unlabeled dye. These data indicate that uptake into the tumor is FAP-mediated. Both targeted and competition subjects showed bioaccumulation in the kidneys.

FIG. 15A-G illustrate a time-course study of a mono-FAP multivalent compound over a 48-h period in mice bearing KB tumors. The mouse at left was given the mono-FAP compound alone, whereas the mouse at right was pretreated with 100-fold excess of an unlabeled FAP ligand. Absence of detectable signal in the pretreated mouse indicated a FAP-mediated retention of the conjugate.

FIG. 16A-D show a comparison between dual-FAP and mono-FAP multivalent compounds bearing a detectable S0456 active agent. The dual-FAP compound was retained beyond 48 hours, whereas the mono-FAP conjugate was nearly undetectable 48 h post-injection.

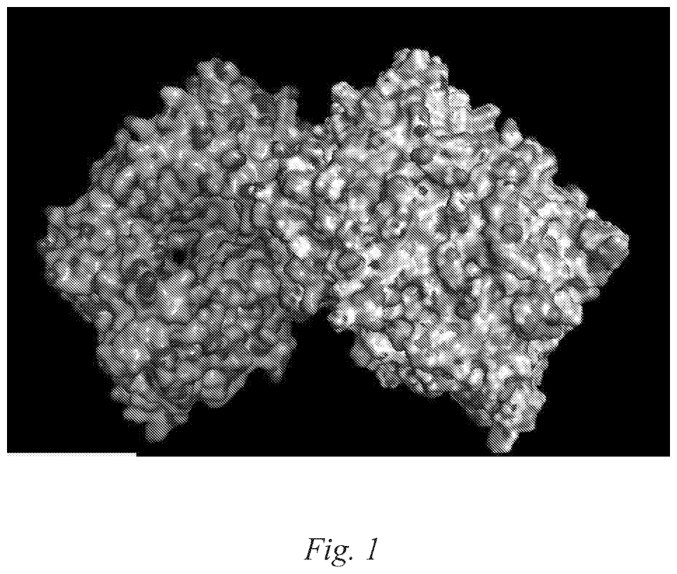

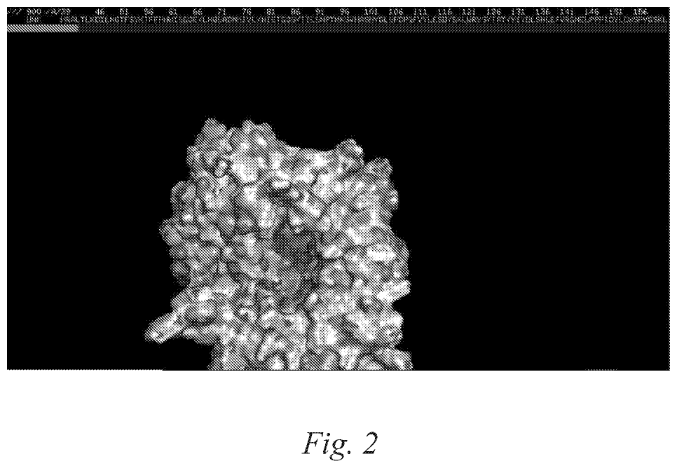

### DETAILED DESCRIPTION

### Definitions

For convenience, before further description, some terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

Some terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer to A only (optionally including elements other than B); or to B only (optionally including elements other than A); or yet, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of”, or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, to at least one, optionally including more than one, A, with no B present (and optionally including elements other than B); or to at least one, optionally including more than one, B, with no A present (and optionally including elements other than A); or yet, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to.

As used herein, the term “administering” includes all means of introducing the compounds and compositions described herein to the host animal, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and/or vehicles.

Unless specifically stated otherwise, the term “about” refers to a range of values plus or minus 10% for percentages and plus or minus 1.0 unit for unit values, for example, about 1.0 refers to a range of values from 0.9 to 1.1.

As used herein, the term “administering” generally refers to any and all means of introducing compounds described herein to the host subject including, but not limited to, by oral, intravenous, intramuscular, subcutaneous, transdermal, inhalation, buccal, ocular, sublingual, vaginal, rectal, and like routes of administration. Compounds described herein may be administered in unit dosage forms and/or compositions containing one or more pharmaceutically acceptable carriers, adjuvants, diluents, excipients, and/or vehicles, and combinations thereof.

Administration of the compounds of the present disclosure as salts may be appropriate. Examples of acceptable salts include, without limitation, alkali metal (for example, sodium, potassium or lithium) or alkaline earth metals (for example, calcium) salts; however, any salt that is generally non-toxic and effective when administered to the subject being treated is acceptable. Similarly, “pharmaceutically acceptable salt” refers to those salts with counter ions which may be used in pharmaceuticals. Such salts may include, without limitation: (1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or (2) salts formed when an acidic proton present in the parent compound either is replaced by a metal ion, e.g., an alkali metal ion, an alkaline earth ion, or an aluminum ion, or coordinates with an organic base such as ethanolamine, diethanolamine, triethanolamine, trimethamine, N-methylglucamine, and the like. Pharmaceutically acceptable salts are well known to those skilled in the art, and any such pharmaceutically acceptable salts may be contemplated in connection with the embodiments described herein.

Acceptable salts may be obtained using standard procedures known in the art, including (without limitation) reacting a sufficiently acidic compound with a suitable base affording a physiologically acceptable anion. Suitable acid addition salts are formed from acids that form non-toxic salts. Illustrative, albeit nonlimiting, examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts of the compounds described herein are formed from bases that form non-toxic salts. Illustrative, albeit nonlimiting, examples include the arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts.

The term “alkyl” denotes a straight chain (i.e. unbranched), branched chain, or a cyclyl arrangement of carbon atoms, or any combination thereof. Alkyl as used herein includes monovalent, divalent, trivalent, or tetravalent radicals. Examples of monovalent hydrocarbon radicals include, without limitation, groups such as methyl, ethyl, n-propyl, isopropyl, cyclopropyl, n-butyl, t-butyl, isobutyl, sec-butyl, cyclobutyl, or homologs and isomers of (e.g., n-pentyl, n-hexyl, n-heptyl, n-octyl, and the like). Examples of divalent radicals include, by way of non-limiting example, methylene, ethylene, propylene, isopropylene, cyclopropylene, and the like. Trivalent and tetravalent alkyls include tri- and tetra-substituted carbon radicals which, upon substitution, form tertiary or quaternary carbon junctions. Alkyl is not limiting to any number of atoms and, unless specifically indicated otherwise, may include a single carbon atom, two carbon atoms, three carbon atoms, four carbon atoms, five carbon atoms, six carbon atoms, or more. Alkyl may be defined within a range, for example, C₁-C₁₀, which indicates anywhere between one and ten carbon atoms are included within the alkyl group. Alkyl may be defined as C₁-C₆, including any orientation of six carbon atoms (e.g., n-hexyl, cyclohexyl, etc.). An alkyl may comprise a plurality of repeating subunits (e.g., polyethylene, polypropylene, etc.). The alkyl can be a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. The alkyl can be optionally substituted with oxo, halogen, —CN, —CF₃, —OH, —OMe, —NH₂, or —NO₂. The alkyl can be optionally substituted with oxo, halogen, —CN, —CF₃, —OH, or —OMe. The alkyl can be optionally substituted with halogen.

The term “alkenyl” denotes a straight chain (i.e. unbranched), branched chain, or a cyclyl arrangement of carbon atoms, or any combination thereof, wherein at least one bond is unsaturated thereby forming a double bond. The group may be in either the cis or trans conformation about the double bond(s) and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. The alkenyl can be a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. An alkenyl can be optionally substituted with oxo, halogen, —CN, —CF₃, —OH, —OMe, —NH₂, or —NO₂. An alkenyl can be optionally substituted with oxo, halogen, —CN, —CF₃, —OH, or —OMe. The alkenyl can be optionally substituted with halogen.

“Alkynyl” refers to an optionally substituted straight-chain or optionally substituted branched-chain hydrocarbon monoradical having one or more carbon-carbon triple-bonds. Examples include, but are not limited to, ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. The alkynyl can be a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. An alkynyl can be optionally substituted with oxo, halogen, —CN, —CF₃, —OH, —OMe, —NH₂, or —NO₂. An alkynyl can be optionally substituted with oxo, halogen, —CN, —CF₃, —OH, or —OMe. The alkynyl can be optionally substituted with halogen.

“Aminoalkyl” refers to an alkyl radical, as defined above, that is substituted by one or more amines. The alkyl can be substituted with one amine. The alkyl can be substituted with one, two, or three amines. Hydroxyalkyl include, for example, aminomethyl, aminoethyl, aminopropyl, aminobutyl, or aminopentyl. The hydroxyalkyl can be aminomethyl.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. The aryl can be a 6- to 10-membered aryl. The aryl can be a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. The aryl can be phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. An aryl can be optionally substituted with halogen, methyl, ethyl, —CN, —CF₃, —OH, —OMe, —NH₂, or —NO₂. An aryl can be optionally substituted with halogen, methyl, ethyl, —CN, —CF₃, —OH, or —OMe. The aryl can be optionally substituted with halogen.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). The cycloalkyl can be a 3- to 6-membered cycloalkyl. The cycloalkyl can be a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Some examples of partially saturated bicyclic cycloalkyls include, by way of non-limiting example, include tetrahydronaphthalene, dihydronaphthalene, indane, indene, and dihydroanthracene. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. A cycloalkyl can be optionally substituted with oxo, halogen, methyl, ethyl, —CN, —CF₃, —OH, —OMe, —NH₂, or —NO₂. A cycloalkyl is can be optionally substituted with oxo, halogen, methyl, ethyl, —CN, —CF₃, —OH, or —OMe. The cycloalkyl can be optionally substituted with halogen.

“Haloalkyl” refers to an alkyl radical, as defined above, that is substituted by one or more halogen atoms. The alkyl is can be substituted with one, two, or three halogen atoms. The alkyl can be substituted with one, two, three, four, five, or six halogens. Haloalkyl includes, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. The haloalkyl can be trifluoromethyl. A divalent alkyl can be substituted with two halogens forming a geminal dihalogen substitution such as —CF₂—, —CCl₂— or the like.

“Halo” or “halogen” refers to bromo, chloro, fluoro or iodo. Halogen can be fluoro or chloro. Halogen can befluoro.

“Heteroalkyl” refers to an alkyl group in which one or more skeletal atoms of the alkyl am selected from an atom other than carbon, e.g., oxygen, nitrogen (e.g., —NH—, —N(alkyl)-), sulfur, or combinations thereof. A heteroalkyl can be attached to the rest of the molecule at a carbon atom of the heteroalkyl. A heteroalkyl can be a C₃-C₆ heteroalkyl wherein the heteroalkyl is comprised of 1 to 5 carbon atoms and one or more atoms other than carbon, e.g., oxygen, nitrogen, sulfur, or combinations thereof. A carbon atom or heteroatom can be optionally oxidized (e.g., —C(O)OCH₂—, —CH₂S(O)₂NHCH₂—, —NHC(O)NHCH₂, —CH₂NHC(O)CH₂). Further examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. A heteroalkyl can be optionally substituted with oxo, halogen, methyl, ethyl, —CN, —CF₃, —OH, —OMe, —NH₂, or —NO₂. A heteroalkyl can be optionally substituted with oxo, halogen, methyl, ethyl, —CN, —CF₃, —OH, or —OMe. The heteroalkyl can be optionally substituted with halogen.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. The heteroaryl can be a 5- to 10-membered heteroaryl. The heteroaryl can be a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. A heteroaryl can be optionally substituted with halogen, methyl, ethyl, —CN, —CF₃, —OH, —OMe, —NH₂, or —NO₂. A heteroaryl is can be optionally substituted with halogen, methyl, ethyl, —CN, —CF₃, —OH, or —OMe. The heteroaryl can be optionally substituted with halogen.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, phosphorous and sulfur. The heterocycloalkyl can comprise 1 or 2 heteroatoms selected from nitrogen and oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₈ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). The heterocycloalkyl can be a 3- to 6-membered heterocycloalkyl. The cycloalkyl can be a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to, the monosaccharides, the disaccharides and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. A heterocycloalkyl can be optionally substituted with oxo, halogen, methyl, ethyl, —CN, —CF₃, —OH, —OMe, —NH₂, or —NO₂. A heterocycloalkyl can be optionally substituted with oxo, halogen, methyl, ethyl, —CN, —CF₃, —OH, or —OMe. The heterocycloalkyl can be optionally substituted with halogen.

A “therapeutically effective amount” or an “effective amount” refers to an amount of a compound administered to a subject (e.g., a mammal, such as a human), either as a single dose or as part of a series of doses, which is effective to produce a desired effect. An effective amount can be an amount required to produce a therapeutic effect. An effective amount can be an amount required to produce an image or other detectable readout. An effective amount can be measured in volume, volume by mass, volume by volume, mass, mass by volume, mass by mass, concentration, radioactivity (e.g., curies, rads, becquerel), or any other metric known in the art.

“Therapeutic agents” comprise any entity capable of producing a desirable physiological response. Therapeutic agents may comprise antifibrotics, anticancer agents, chemotherapeutics, radiotherapeutics, or the like.

“Treatment” of a subject (e.g., a mammal, such as a human) includes any type of intervention used in an attempt to alter the natural course of the subject. Treatment can include administration of a pharmaceutical composition, subsequent to the initiation of a pathologic event or contact with an etiologic agent and includes stabilization of the condition (e.g., condition does not worsen, e.g., cancer does not metastasize and the like) or alleviation of the condition (e.g., reduction in tumor size, remission of cancer, absence of symptoms of autoimmune disease and the like). In other embodiments, treatment also includes prophylactic treatment (e.g., administration of a composition described herein when an individual is suspected to be suffering from a condition described herein).

As used herein, “subject”, “individual” and “patient” are used interchangeably. None of the terms imply that a medical professional is required for the administration of the compounds disclosed herein. Any of these terms refer to a mammal. The mammal can be a human.

The terms “multivalent conjugate,” “conjugate,” “multivalent compound,” and “compound” may be used interchangeably unless specified otherwise.

### Multivalent Compounds

The disclosure relates to a multivalent compound comprising two or more FAP-binding ligands (also referred to as “targeting ligands,” or “Q”) conjugated to a multipoint template. The two or more FAP-binding ligands can be configured to bind the two dimeric chains of FAP in its active form. Activation of the dimeric FAP can lead to internalization of the entire complex. In addition to comprising one or more FAP-binding ligands, the multipoint template can be further functionalized with an active agent (“X”). An active agent can be more advantageously utilized inside a FAP-expressing cell than when disposed in the extracellular space. In alternative embodiments, the compound is not internalized and remains disposed in the extracellular space. A multivalent compound can further comprise one or more spacers. A spacer may, for example, orient the FAP-binding ligands and/or the active agent in a desired conformation or spatial arrangement. A spacer and the FAP-binding ligand or active agent to which the spacer is attached can be referred to as an “arm.” A multivalent compound can have multiple FAP-binding or active agent arms. A multivalent compound can have two, three, four, five, or six FAP-binding arms, alternatively referred to as Q-arms. The spacer conjoining the FAP-binding ligand Q to the multipoint template may be referred to herein as a Q-spacer. In other embodiments, a multivalent compound can comprise one or more active agents (“X”) linked to the multipoint template via a spacer, sometimes referred to herein as X-arms, wherein the spacer itself may be referred to as an X-spacer. One, two, three, or more X-arms can be disposed within a multivalent compound.

A FAP-binding ligand, Q, can be any agent that binds to a fibroblast activation protein (e.g., fibroblast activation protein alpha) with an affinity (e.g., K_{D}, EC₅₀) of 1 micromolar (μM) or greater. It should be noted that greater/higher affinity is associated with a lower dissociation constant (K_{D}) or effective concentration that gives a half-maximal response (EC₅₀). A FAP-binding ligand may have a K_{D} or EC₅₀ of 10 μM or less (e.g., 5 μM, 2 μM, 1 μM, 500 nanomolar (nM), 200 nM, 100 nM, 50 nM, 20 nM, 10 nM, 5 nM, 1 nM, 500 picomolar (pM), 200 pM, 100 pM, etc.). A FAP-binding ligand may have a K_{D} or EC₅₀, of 1 pM or greater (e.g., 2 pM, 5 pM, 10 pM, 20 pM, 50 pM, 100 pM, 200 pM, 500 pM, 1 nM, 2 nM, 5 nM, 10 nM, 20 nM, 50 nM, 100 nM, 200 nM, 500 nM, 1 μM, 2 μM, 5 μM, etc.). Binding of a first FAP-binding ligand can facilitate the binding of a subsequent (e.g., second, third, etc.) FAP-binding ligand. The binding of two or more FAP-binding ligands can be cooperative. The binding of a first FAP-binding ligand can increase the effective concentration locally. The two or more FAP-binding ligands can bind to two chains of a FAP dimer. Three or more FAP-binding ligands can bind to two chains of a FAP trimer. Four or more FAP-binding ligands can bind to four chains of a FAP tetramer. Only one FAP-binding ligand can bind to a target FAP.

A FAP-binding ligand, Q, can be any agent that associates with FAP. By way of non-limiting example, Q can be a molecule (e.g., small molecule, macromolecule, tethered molecule), an amino acid (e.g., a natural amino acid, an unnatural amino acid, a functionalized amino acid), a peptide (e.g., a polypeptide, a natural peptide, an unnatural peptide, a linear peptide, a cyclic peptide, and the like), or any combination thereof. Q can comprise a pyrrolidine derivative. Q can be a cyanopyrrolidine derivative. Q can be a fluoropyrrolidine derivative. Q can comprise a geminal difluorinated pyrrolidine.

Q can have the structure:

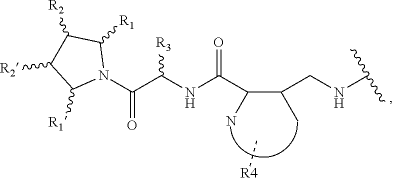

wherein each R¹ and R^{1′} is independently hydrogen, alkyl, aryl, —CN, —COOH, —B(OH)₂, SO₃H, or PO₃H; each R² and R^{2′} is independently hydrogen, halogen, alkyl, aryl, or heteroaryl; R³ is hydrogen, alkyl, alkenyl, aryl, or heteroaryl;

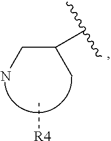

also referred to throughout the disclosure as “Ring A”, is a 3 to 10-membered heterocycle or 5 to 10-membered heteroaryl, wherein each heterocycle or heteroaryl can contain one or more N atoms and is substituted with R⁴; and R⁴ is hydrogen, halogen, alkyl, alkenyl, aryl, or heteroaryl.

Each R² and R^{2′} can independently be hydrogen, halogen, or alkyl. Each R² and R^{2′} can independently be hydrogen, halogen, or alkyl. Each R² and R^{2′} can independently be hydrogen, or halogen. Two R² or two R^{2′} groups can be hydrogen, halogen, alkyl, or haloalkyl. Two R² or two R^{2′} groups can be hydrogen, halogen, or alkyl. Two R² or two R^{2′} groups can be hydrogen. Two R² or two R^{2′} groups can be halogen. Two R² or two R^{2′} groups can be alkyl. Tach R² and R^{2′} can be independently hydrogen, fluorine, or chlorine. Each R² and R^{2′} can be independently hydrogen, fluorine, or chlorine. Each R² and R^{2′} can independently be hydrogen or fluorine. Each R² and R^{2′} can independently be fluorine or chlorine. Two R² or two R^{2′} groups can be hydrogen, fluorine, or chlorine. Two R² groups can be hydrogen. Two R² groups can be fluorine. Two R² groups can be chlorine. Two R^{2′} groups can be hydrogen. Two R^{2′} groups can be fluorine. Two R^{2′} groups can be chlorine.

Each R¹ and R^{1′} can be independently hydrogen, alkyl, aryl, —CN, —COOH, —B(OH)₂, SO₃H, or PO₃H. Each R¹ and R^{1′} can be independently hydrogen, alkyl, aryl, —CN, —COOH, or —B(OH)₂. Each R¹ and R can be independently hydrogen, alkyl, —CN, or —B(OH)₂. Each R¹ and R^{1′} can be independently hydrogen, —CN, or —B(OH)₂. R¹ can be hydrogen and R^{1′} can be —CN or —B(OH)₂. R¹ can be hydrogen and R^{1′} can be —B(OH)₂. R¹ can be hydrogen and R^{1′} can be —CN. R^{1′} can be in the (S) stereochemical configuration. R^{1′} can be in the (R) stereochemical configuration.

R; can be hydrogen, alkyl, haloalkyl, alkenyl, aryl, or heteroaryl. R³ can be hydrogen, alkyl, alkenyl or aryl. R³ can be hydrogen, alkyl, or alkenyl. R³ can be hydrogen or alkyl. R³ can be hydrogen or CH₃. R³ can be CH₃. R³ can be in the (S) stereochemical configuration. R³ can be in the (R) stereochemical configuration. R³ can be hydrogen.

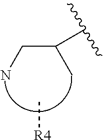

(“Ring A”) can be a 3 to 10-membered heterocycle or 5 to 10-membered heteroaryl, wherein each heterocycle or heteroaryl can contain one or more N atoms and is optionally substituted with R⁴. Ring A can be an optionally substituted 5 to 10-membered heteroaryl. Ring A can be an optionally substituted monocyclic 5 or 6-membered heteroaryl. Ring A can be an optionally substituted bicyclic 9 or 10-membered heteroaryl. Ring A can be an optionally substituted pyridinyl, pyrimidinyl, pyrazinyl, pyridazinyl, triazinyl, quinolinyl, naphthyridinyl, pyridopyrazinyl, pyridopyrimidinyl, tetrahydroquinolinyl, dihydronaphthyridinyl, dihydropyridopyrazinyl, dihydropyridopyrimidinyl, triazolopyridinyl, pyrazolopyridinyl, pyrrolopyridinyl, imidazopyridinyl, indazolyl, indolyl, isoindolyl, oxazolopyridinyl, thiadiazolopyridinyl, pyrrolyl, pyrazolyl, triazolyl, imidazolyl, oxazolyl, isoxazolyl, oxadiazolyl, thiazolyl, isothiazolyl, or thiadiazolyl, Ring A can be pyridinyl, quinolinyl, naphthyridinyl, pyridopyrazinyl, pyridopyrimidinyl, tetrahydroquinolinyl, dihydronaphthyridinyl, dihydropyridopyrazinyl, dihydropyridopyrimidinyl, triazolopyridinyl, pyrazolopyridinyl, pyrrolopyridinyl, imidazopyridinyl, oxazolopyridinyl, thiadiazolopyridinyl, pyrrolyl, pyrazolyl, triazolyl, imidazolyl, oxazolyl, or thiazolyl, Ring A can be selected from the following group of radicals:

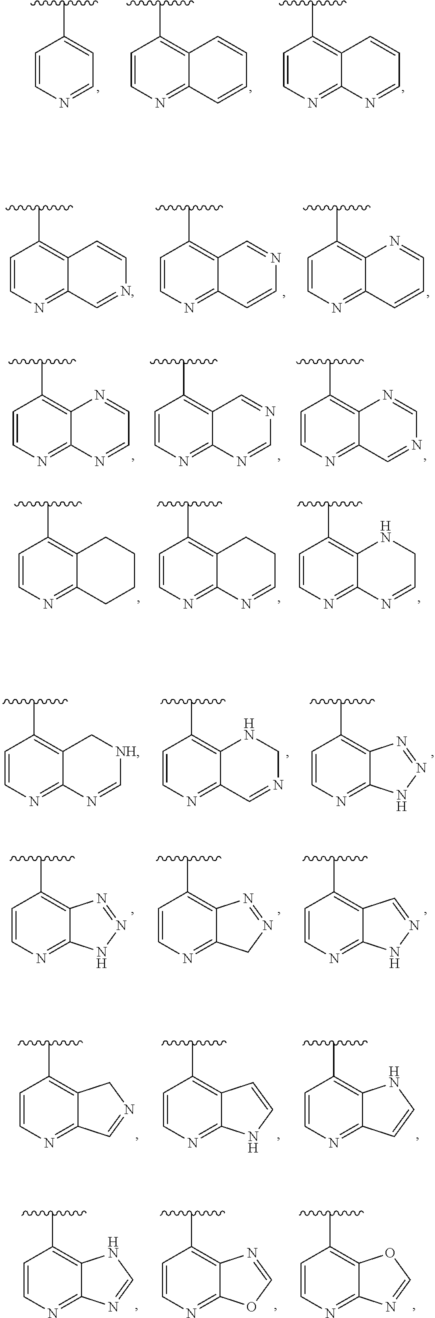

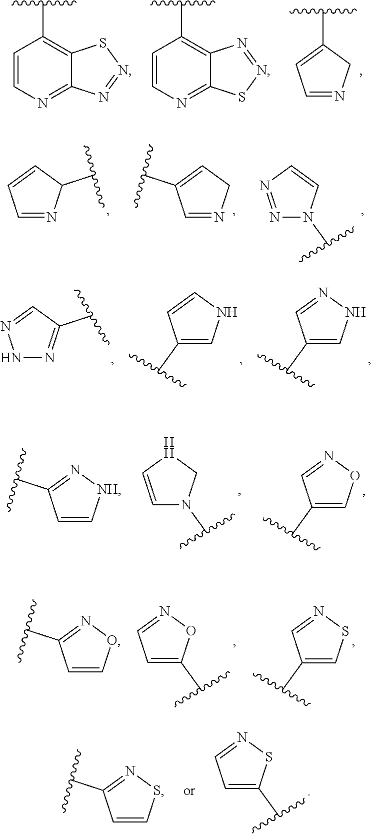

Ring A can be an optionally substituted pyridinyl, pyrimidinyl, pyrazinyl, pyridazinyl, quinolinyl, naphthyridinyl (e.g., 1,8-naphthyridinyl, 1,7-naphthyridinyl, 1,6-naphthyridinyl, 1,5-naphthyridinyl), pyrrolopyridinyl, pyrazolopyridinyl, pyrrolyl, pyrazolyl, triazolyl, or imidazolyl. Ring A can be an optionally substituted pyridinyl, pyrimidinyl, pyrazinyl, pyridazinyl, pyrrolyl, pyrazolyl, triazolyl, or imidazolyl. Ring A is an optionally substituted pyridinyl, pyrimindinyl, pyrazinyl, or pyridazinyl. Ring A is an optionally substituted pyridinyl or pyrimidinyl. Ring A can be an optionally substituted pyrimidinyl. Ring A can be an optionally substituted pyridinyl.

R⁴ can be hydrogen, halogen, alkyl, alkenyl, aryl, or heteroaryl. R⁴ can be hydrogen, halogen, or alkyl. R⁴ can be halogen. R⁴ can be H, F, Cl, Br, I, methyl, ethyl, propyl, isopropyl, cyclopropyl, CF₃, CHF₂, or CH₂F. R⁴ can be H, F, Cl, Br, CH₃, or CF₃. R⁴ can be Cl, CH₃, or CF₃. R⁴ can be Cl or CH₃. R⁴ can be Cl or CF₃. R⁴ can be CH₃ or CF₃. R⁴ can be Cl.

Ring A can be a chloropyridine. Ring A can be a 2-chloropyridine. Ring A can be a 3-chloropyridine. Ring A can be a 4-chloropyridine. Ring A can be a 5-chloropyridine. Ring A can be a 6-chloropyridine.

Q can comprise one or more structures of Table 1.

TABLE 1
FAP-Targeting Ligands Q

| FAP | |
|---|---|
| ligand | Structure |
| Q^{A} | 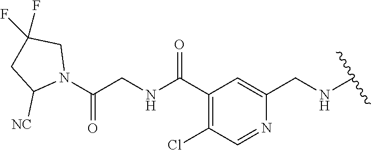 [SMILES: *NCc1cc(C(=O)NCC(=O)N2CC(F)(F)CC2C#N)c(Cl)cn1] |
| Q^{B} | 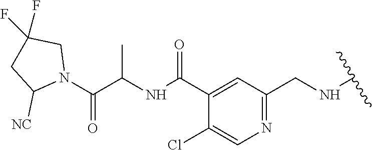 [SMILES: *NCc1cc(C(=O)NC(C)C(=O)N2CC(F)(F)CC2C#N)c(Cl)cn1] |
| Q^{C} | 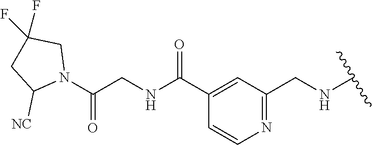 [SMILES: *NCc1cc(C(=O)NCC(=O)N2CC(F)(F)CC2C#N)ccn1] |
| Q^{D} | 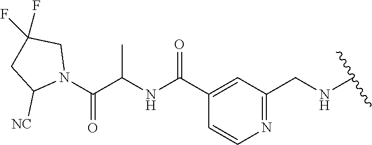 [SMILES: *NCc1cc(C(=O)NC(C)C(=O)N2CC(F)(F)CC2C#N)ccn1] |

A multivalent conjugate can have the same FAP-binding ligand for each Q. Each Q is a different FAP-binding ligand. A multivalent compound can have two identical FAP-binding ligands (Q¹) and one or more additional FAP-binding ligands (Q²). Two Q ligands can be stereoisomers or regioisomers of one another. Each Q can have the same spacer L^{Q}. Each Q can have a different spacer (e.g., L^{Q1}, L^{Q2}, etc.). Two or more Q can have the same spacer L^{Q1} while one or more additional Q can have a different spacer L^{Q2}.

One or more Q can be replaced with W, provided that two or more Q are not W. One or more Q can be replaced with W, provided that two Q are FAP-binding ligands. W can comprise a solubility enhancer or PK/PD modulator. W can comprise a polyethylene glycol (PEG), sugar, peptide, or peptidoglycan. W can comprise a PEG, sugar, peptide, or peptidoglycan for achieving better solubility and PK/PD properties. W can comprise one or more monosaccharide, disaccharide, peptide, peptidoglycan, and/or serum albumin. W can comprise one or more PEG, peptide, peptidoglycan, or serum albumin. W might not comprise a sugar. W might not comprise a monosaccharide, disaccharide, or polysaccharide. W may not comprise a glycan. W can comprise a glycosylated amino acid. W can comprise a glycosylate cysteine. W can comprise a free carboxylic acid. W can comprise a PEG.

A spacer “L” can comprise any stable arrangement of atoms. A spacer comprises one or more L′. Each L′ is independently selected from the group consisting an amide, ester, urea, carbonate, carbamate, disulfide, amino acid, amine, ether, alkyl, alkene, alkyne, heteroalkyl (e.g., polyethylene glycol), cycloakyl, aryl, heterocycloalkyl, heteroaryl, carbohydrate, glycan, peptidoglycan, polypeptide, or any combination thereof. Any spacer can comprise any one or more of the following units: an amide, ester, urea, carbonate, carbamate, disulfide, amino acid, amine, ether, alkyl, alkene, alkyne, heteroalkyl (e.g., PEG), cycloakyl, aryl, heterocycloalkyl, heteroaryl, carbohydrate, glycan, peptidoglycan, polypeptide, or any combination thereof. A spacer L or L′ can comprise a solubility enhancer or PK/PD modulator W. A spacer can comprise a glycosylated amino acid. A spacer can comprise one or more monosaccharide, disaccharide, polysaccharide, glycan, or peptidoglycan. A spacer can comprise a releasable moiety (e.g., a disulfide bond, an ester, or other moieties that can be cleaved in vivo). A spacer can comprise one or more units such as ethylene (e.g., polyethylene), ethylene glycol (e.g., PEG), ethanolamine, ethylenediamine, and the like (e.g., propylene glycol, propanolamine, propylenediamine). A spacer can comprise an oligoethylene, PEG, alkyl chain, oligopeptide, polypeptide, rigid functionality, peptidoglycan, oligoproline, oligopiperidine, or any combination thereof. A spacer can comprise an oligoethylene glycol or a PEG. A spacer can comprise an oligoethylene glycol. A spacer can comprise a PEG. A spacer can comprise an oligopeptide or polypeptide. A spacer can comprise an oligopeptide. A spacer can comprise a polypeptide. A spacer can comprise a peptidoglycan. A spacer might not comprise a glycan. A spacer might not comprise a sugar. A rigid functionality can be an oligoproline or oligopiperidine. A rigid functionality can be an oligoproline. A rigid functionality can be an oligopiperidine. A rigid functionality can be an oligophenyl. A rigid functionality can be an oligoalkyne. An oligoproline or oligopiperidine can have about two up to and including about fifty, about two to about forty, about two to about thirty, about two to about twenty, about two to about fifteen, about two to about ten, or about two to about six repeating units (e.g., prolines or piperidines).

A spacer (e.g., L^{Q} or L^{X}) can comprise one or more of the following units:

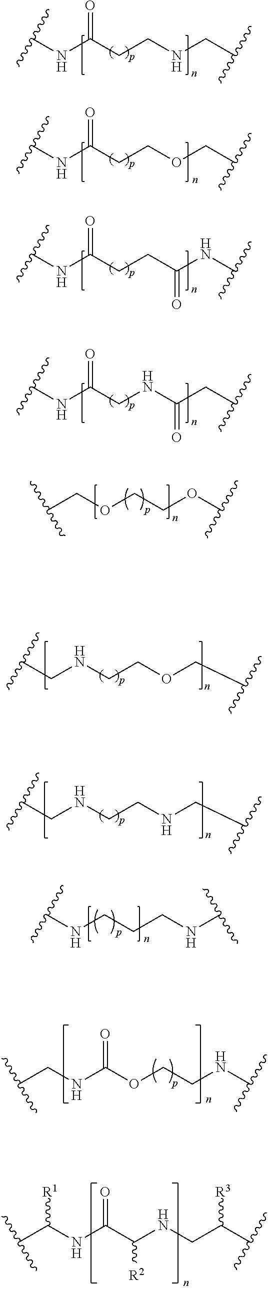

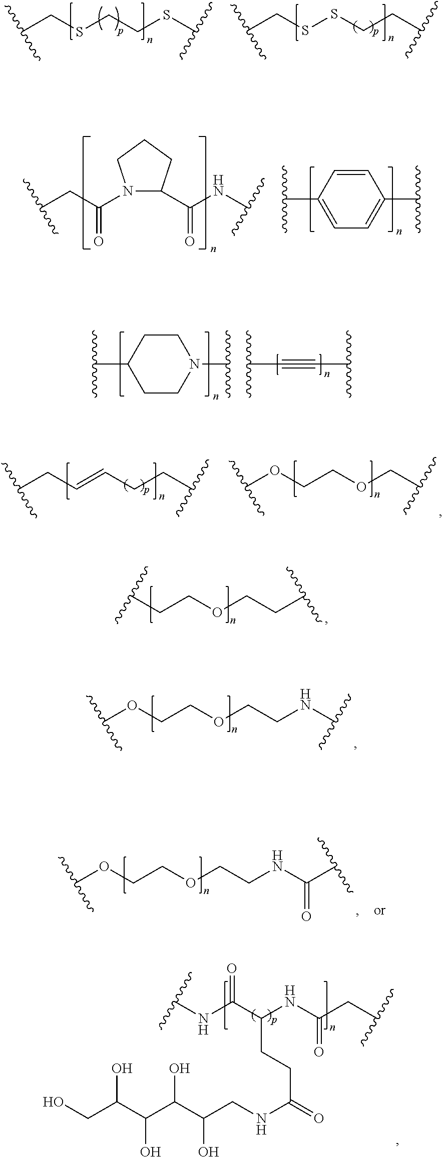

or any combination thereof, where p is an integer between 0 and about 20, and n is an integer between 1 and about 32. A spacer can comprise the structure:

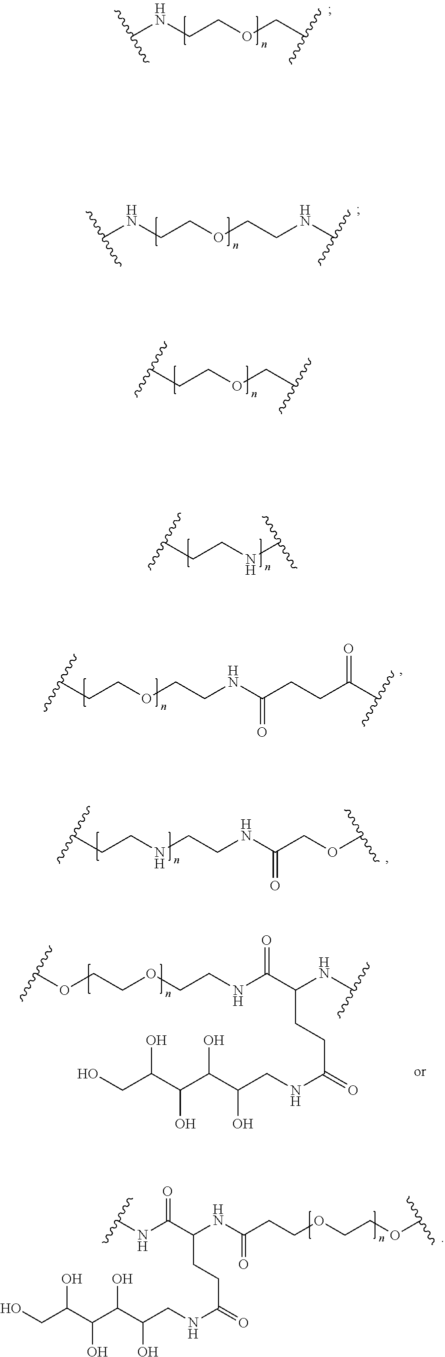

A Q-spacer can comprise one or more structures described in Table 2.

TABLE 2
Q-spacers.

| Q-spacer | Structure |
|---|---|
| L^{QA} | 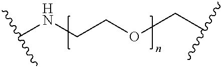 |
| L^{QB} | 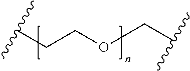 |
| L^{QC} | 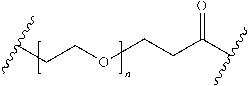 |
| L^{QD} | 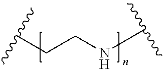 |

A spacer (e.g., L^{X} or L^{Q}) can have a length of 5 angstroms (Å), 10 Å, 15 Å, 20 Å, 50 Å, 100 Å, 200 Å, 300 Å, or more. A spacer can have a length of 300 Å, 200 Å, 100 Å, 50 Å, 20 Å, 15 Å, 10 Å, 5 Å, or less. In the following list, ranges should be understood to be inclusive of the upper and lower limits (e.g., 1 to 3 includes 1, 2, and 3). A spacer can have a length of about 10 to about 300 Å, of about 10 to about 200 Å, of about 10 to about 100 Å, of about 10 to about 50 Å, of about 15 to about 300 Å, of about 15 to about 200 Å, of about 15 to about 150 Å, of about 15 to about 100 Å, of about 20 to about 300 Å, of about 20 to about 200 Å, of about 20 to about 100 Å. A spacer (e.g., L^{Q} or L^{X}), can have a length of about 10 to about 300 Å, of about 10 to about 200 Å, or of about 10 to about 100 Å. A spacer (e.g., L^{Q} or L^{X}) can have a length of about 15 to about 300 Å, of about 15 to about 200 Å, or of about 15 to about 100 Å. A spacer (e.g., L^{Q} or L^{X}) can have a length of about 20 to about 300 Å, of about 20 to about 200 Å, or of about 20 to about 100 Å. A spacer (e.g., L^{Q} or L^{X}) can have a length of about 15 to about 200 Å.

A spacer can orient two or more units (e.g., active agents, targeting ligands, multipoint templates) in a particular orientation or distance. For example, a spacer can separate a targeting ligand and an active agent by a particular distance, or a spacer can orient a targeting ligand and an active agent in a particular spatial arrangement or conformation. A spacer can separate two targeting ligands by a particular distance, or a spacer can orient two targeting ligands in a particular spatial arrangement or conformation. A spacer can separate a targeting ligand or an active agent from the multipoint template by a particular distance, or a spacer can orient a targeting ligand or an active agent in a particular spatial arrangement or conformation in relation to the multipoint template. A spacer can contain a combination of flexible and rigid elements (e.g., a polypeptide spacer, a polyester spacer, an oligopiperidine spacer, an oligoproline spacer). A spacer can also contain conformational restrictions. A spacer can be substantially straight (e.g., a polyalkyne or a polyphenyl spacer). A spacer can have a particular shape (e.g., C-shaped, V-shaped, L-shaped, S-shaped, helical).

A spacer can provide additional functions of utility besides spacing. For example, a spacer may modulate (e.g., increase, decrease, enhance, mitigate, optimize) certain properties of a multivalent compound or a portion thereof. For example, a spacer can modulate physicochemical, pharmacological, pharmacodynamic, pharmacokinetic, biophysical, biological, physical, or commercial properties. A spacer can comprise a trivalent linker, in which the third position of the linker of the ligand-drug compound is the free —COOH of the cysteine in the linker. The —COOH group can be used to attach (e.g., at position W described herein) a PEG compound, a sugar, a peptide, a peptidoglycan, or a serum albumin. A linker can comprise a PEG compound, a peptide, a peptidoglycan, or a serum albumin. A spacer might not comprise a sugar. A spacer may modulate plasma protein binding, membrane permeability, solubility, lipophilicity, polar surface area, total surface area, size, mass, non-covalent bonding (e.g., hydrogen bonding, ionic bonding, Van der Waals interactions), ionization (e.g., acidity, basicity), metabolism, conjugation, excretion, retention, or any combination thereof. A spacer may enhance residence time or internalization by modulating one or more factors (e.g., permeability, lipophilicity, or protein binding). A spacer can comprise one or more groups that imparts a desired effect. For example, a spacer can comprise a substrate to facilitate active transport into a cell. A spacer can reduce excretion by enhancing plasma protein binding. A spacer can contain one or more sugar moieties, or one or more proteins or protein fragments (i.e., peptides, polypeptides). A spacer can comprise one or more carbohydrate moieties (e.g., lipids, fatty acids). A spacer can be glycosylated (e.g., containing one or more monosaccharide, disaccharide, polysaccharide, glycan, or glycogens). A spacer can comprise a glycosylated amino acid.

A spacer L^{X} can comprise a divalent radical as indicated in Table 3, wherein “Y” and “X” denote attachments to a multipoint template and active agent, respectively.

TABLE 3
X-Spacers

| X-spacer | |
|---|---|
| name | Structure |
| L^{XA} | 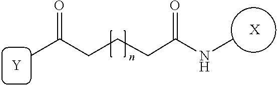 |
| L^{XB} | 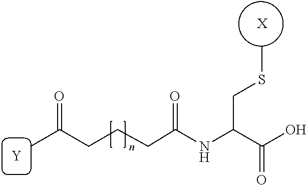 |
| L^{XC} | 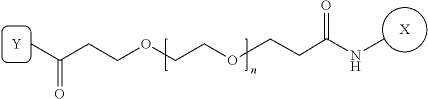 |
| L^{XD} | 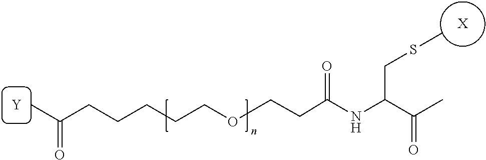 |
| L^{XE} | 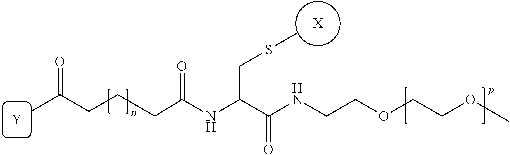 |
| L^{XF} | 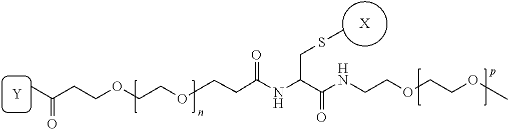 |
| L^{XG} | 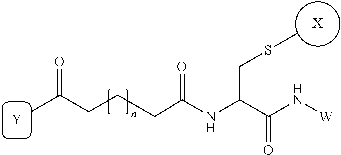 |
| L^{XH} | 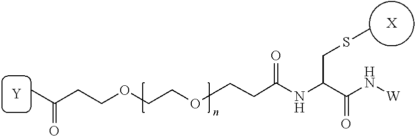 |
| L^{XI} | [symbol] |

[symbol]

indicates data missing or illegible when filed

wherein n and pare integers from 0 up to and including 100, and W comprises one or more monosaccharide, disaccharide, peptide, peptidoglycan, solubility enhancer, PK/PD modulator, or a combination thereof, and X and Y are shown solely to note a connection to X and Y; it should be understood that X and Y are not part of L^{X}.

W can comprise one or more monosaccharide, disaccharide, oligosaccharide, polysaccharide, peptide, peptidoglycan, serum albumin, solubility enhancer, PK/PD modulator, or a combination thereof. W can modulate a pharmacological, pharmacokinetic, pharmacodynamic, or physicochemical property. W can facilitate internalization. W can improve aqueous solubility. W can increase plasma protein binding. W can modulate (e.g., reduce) the compound's excretion, elimination, metabolism, stability (e.g., enzymatic stability, plasma stability), distribution, toxicity, or a combination thereof.

A monosaccharide such as found in W can exist in an equilibrium between its linear and cyclic form. A monosaccharide can be linear. A monosaccharide can be cyclic. A monosaccharide can exist as a D isomer. A monosaccharide can exist as an L isomer. As non-limiting examples, W can comprise one or more monosaccharides selected from the following: ribose, galactose, mannose, glucosefructose, N-acetylglucosamine, N-acetylmuramic acid or derivatives thereof (e.g., cyclic or linear forms, methylated derivatives, acetylated derivatives, phosphorylated derivatives, aminated derivatives, oxidized or reduced derivatives, D or L isomers, isotopes, stereoisomers, regioisomers, tautomers, or combinations thereof).

A disaccharide, oligosaccharide, or polysaccharide, as may be disposed within W, can contain an O-linkage, an N-linkage, a C-linkage, or a combination thereof. A disaccharide, oligosaccharide, or polysaccharide may contain a glycosidic linkage in either an α- or β-orientation. W can comprise an oligosaccharide, a polysaccharide, or a glycan (e.g., a glycoprotein, glycopeptide, glycolipid, glycogen, proteoglycan, peptidoglycan, and the like).

W can comprise an amino acid, a peptide, a polypeptide, or a protein. An amino acid can be a natural amino acid (e.g., alanine (Ala), arginine (Arg), asparagine (Asn), aspartic acid (Asp), cysteine (Cys), glutamic acid (Glu), glutamine (Gln), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val)). Alternatively, an amino acid can be an unnatural or modified amino acid. W can comprise a sugar or sugar derivative covalently attached to the side chain of an amino acid (e.g., a glutamic acid, an aspartic acid).

W can comprise a glycosylated amino acid such as:

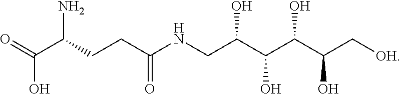 [SMILES: N[C@H](CCC(=O)NC[C@H](O)[C@@H](O)[C@H](O)[C@H](O)CO)C(=O)O]

A peptide or polypeptide can be comprised of a plurality of amino acids, natural and/or unnatural. A peptide (or peptidoglycan) can have about two and about twenty amino acids. An amino acid, a peptide, a polypeptide, or a protein (e.g., such as disposed within or making up W) can have a pharmacological of physicochemical effect that enhances one or more properties of the compound (e.g., modulating solubility, size, permeability, protein binding, target binding, excretion, metabolism, toxicity, distribution, half-life, and/or duration of action). W can be a pharmacokinetic modulator. The pharmacokinetic modulator can be a peptide or protein that can modulate (e.g., enhancing) protein binding. The pharmacokinetic modulator can enhance plasma protein binding. The pharmacokinetic modulator reduces the rate of elimination, excretion, or metabolism. The pharmacokinetic modulator can increase the duration of action of the compound.

A spacer L^{Q} or L^{X}, along with the corresponding targeting ligand Q or active agent X, may be referred to as Q-arms, X-arms, or collectively as “arms.” A multivalent compound having three, four, five, six, seven, eight, nine, or more arms is provided. A multivalent compound can have two Q-arms and one X-arm. A multivalent compound can have three Q-arms and one X-arm. A multivalent compound can have four Q-arms and one X-arm. A multivalent compound can have six Q-arms and one X-arm. A multivalent compound can have two Q-arms and two X-arms. Q can be replaced with W, provided two or more Q are not W (e.g., one Q is W, two Q are FAP-binding ligands).

As described herein, two to six Q-arms and one or more X-arms are conjoined at a juncture “Y” also referred to as a multipoint template. A multipoint template is a molecular construct that can be functionalized (e.g., with Q-arms and X-arms). Such a multipoint template can, by way of non-limiting example, comprise one or more amine, amide, alcohol, ester, acid, alkyne, azide, triazole, heterocycle, boronic acid, halide, electrophile, nucleophile, or additional functional group that participate in conjugation (e.g., via amide coupling, ester synthesis, click chemistry, Suzuki, Negishi, Buchwald, Chan-Lam, Ulman, or other related chemical transformations for joining two groups). A multipoint template can contain a plurality of amines. A multipoint template contains a plurality of amides. A multipoint template can contain a plurality of ethers. A multipoint template Y can comprise a tri-acid-based template, an oligolysine-based template, a Trebler phosphoramidite template, an oligo-hydroxyprolinol-based template, a tris (2-amino-2-(hydroxymethyl)-1,3-propanediol)-based template, a citric acid-based template, a tert-butyl (2-(3,5-diethynylbenzamido)ethyl)carbamate template, or a N-(2-aminoethyl)-3,5-di(1H-1,2,3-triazol-5-yl)benzamide template. A multipoint template Y can comprise a tri-acid-based template, an oligolysine-based template, a Trebler phosphoramidite template, or an oligo-hydroxyprolinol-based template. A multipoint template Y can comprise a tris (2-Amino-2-(hydroxymethyl)-1,3-propanediol)-based template. A multipoint template Y can comprise a structure as described in Table 4.

TABLE 4
Multipoint Templates

| X-spacer | |
|---|---|
| name | Structure |
| Y^{A} | 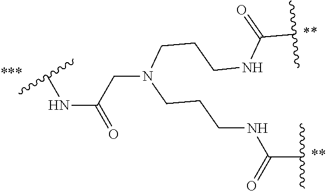 [SMILES: *NC(=O)CN(CCCNC(*)=O)CCCNC(*)=O] |
| Y^{B} | 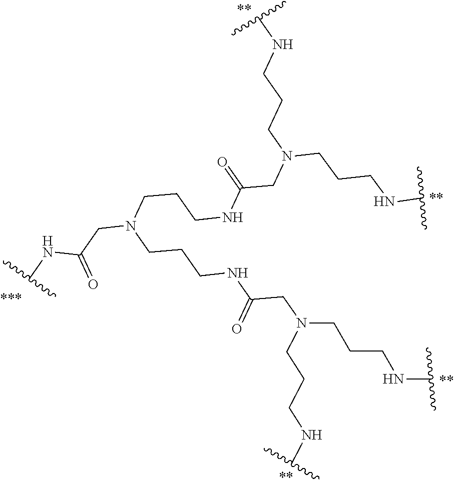 [SMILES: *NCCCN(CCCN*)CC(=O)NCCCN(CCCNC(=O)CN(CCCN*)CCCN*)CC(=O)N*] |
| Y^{C} | 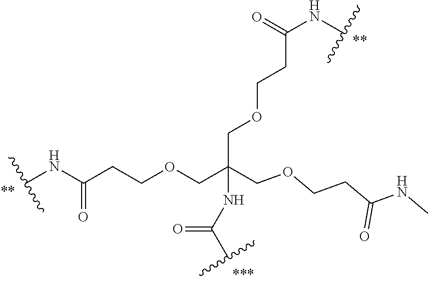 [SMILES: *NC(=O)CCOCC(COCCC(=O)N*)(COCCC(=O)NC)NC(*)=O] |
| Y^{D} | 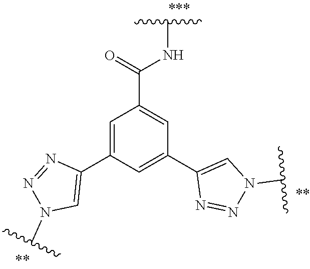 [SMILES: *NC(=O)c1cc(-c2cn(*)nn2)cc(-c2cn(*)nn2)c1] |
| Y^{E} | 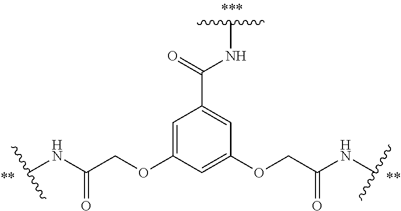 [SMILES: *NC(=O)COc1cc(OCC(=O)N*)cc(C(=O)N*)c1] |
| Y^{F} | 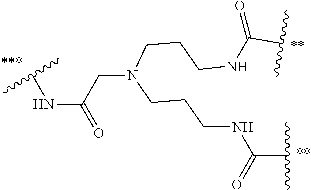 [SMILES: *NC(=O)CN(CCCNC(*)=O)CCCNC(*)=O] |

wherein ** represents attachment between Y and L^{Q}, and *** represents an attachment
between Y and L^{X}.

A multipoint template Y can comprise a di-acid-based template. A multipoint template Y can comprise a tri-acid-based template. A multipoint template Y can comprise a tetra-acid-based template. A multipoint template Y can comprise an oligolysine-based template. A multipoint template Y can comprise a Trebler phosphoramidite template. A multipoint template Y can comprise an oligo-hydroxyprolinol-based template. A multipoint template can contain a different Q in each Q-arm. A multipoint template can contain two or more of the same Q in corresponding two or more Q-arms and at least one additional (i.e., different) Q in the corresponding at least one Q-arm(s). A multipoint template Q is connected to two Q via a Q-spacer comprising a PEG moiety. A multipoint template can be trivalent. A multipoint template can be tetravalent. A multipoint template can be pentavalent. A multipoint template can be hexavalent. A multipoint template or spacer attached thereto can comprises a releasable moiety (e.g., a disulfide bond, an ester) that can be cleaved in vivo.

### Active Agents

As described throughout the specification, a multivalent compound can contain a variety of different active agents (“X”). For example, X can be a detectable agent (e.g., fluorescent dye, a near-infrared (NIR) dye, radio-imaging agent, chelating agent), or a therapeutic agent (e.g., a drug, a photodynamic therapeutic agent, a radiotherapeutic agent, a chemotherapeutic agent, an antifibrotic agent, an anticancer agent, a chelating agent). X can be any entity (e.g., a detectable or therapeutic agent) useful in the detection or treatment of a tumor. X can be effective in both the detection and the treatment of a tumor. X can be utilized to detect or treat a fibrotic tissue. X can be used to treat or detect any cell (e.g., a fibroblast or CAF) expressing fibroblast activation protein (“FAP”). X can be a detectable agent. X can be a therapeutic agent. X can be a fluorescent dye or radio-imaging agent. X can be a photodynamic therapeutic agent. X can be a radiotherapeutic agent. X can reduce or abrogate a fibroblast's ability to synthesize or transport extracellular matrix components (e.g., collagens, elastin, glycosaminoglycans, proteoglycans (e.g., perlecan), and glycoproteins). X can be effective against cancer cells, cancer-associated fibroblasts (CAFs), a tumor microenvironment factor (e.g., a growth factor (e.g., vascular endothelial growth factor (VEGF), basic fibroblast growth factor (bFGF), insulin-like growth factors 1 and 2 (IGF1 and IGF2), transforming growth factor-β (TGF-β), epidermal growth factor (EGF), heparin-binding EGF-like growth factor (HB-EGF), and tumor necrosis factor (TNF)), a hormone, a signaling molecule, an angiogenesis stimulator, a lysyl oxidase (LOX), collagens, elastin, glycosaminoglycans, glycoproteins, or proteoglycans (e.g., perlecan)).

An active agent can be a fluorescent dye. X can be a fluorescent dye with an excitation and/or emission wavelength in the range of 200-1.000 nm, 200-800 nm, 300-1.000 nm, 300-800 nm, 400-1.000 nm, 400-800 nm, 500-1,000 nm, or 500-800 nm. X can be a fluorescent dye with an excitation and/or emission wavelength in the range of 200-1,000 nm. X can be a fluorescent dye with an excitation or emission wavelength in the range of 400-1,000 nm. X can be a fluorescent dye with an excitation or emission wavelength in the range of 400-800 nm. X can be a fluorescent dye with an excitation or emission wavelength in the range of 500-800 nm. X can be a fluorescent dye with an excitation or emission wavelength in the range of 500-700 nm. X can be a fluorescent dye with an excitation or emission wavelength in the range of 650-1.050 nm. X can be a fluorescent dye with an excitation or emission wavelength in the range of 650-850 nm. X can be a fluorescent dye with an excitation or emission wavelength in the range of 650-750 nm. X can be a fluorescent dye with an excitation or emission wavelength in the visible light range (e.g., about 400 to about 800 nm, or about 380 to about 740 nm). X can be a fluorescent dye with an excitation or emission wavelength in the near infrared (“NIR”) range (e.g., about 750 to about 1.400 nm).

A conjugate can include a detectable agent, such as a near infrared (NIR) dye or a radioactive imaging agent. Representative compounds that may be used as detectable agents in accordance with the present teachings include, but are not limited to, dyes (e.g., Rhodamine dyes, cyanine dyes, fluorescein dyes, etc.), positron emission tomography (PET) imaging agents, radiolabeled agents, and the like. Representative examples of Rhodamine dyes include, but are not limited to, Rhodamine B, Rhodamine 6G, Rhodamine 123, and the like. X can be a Rhodamine dye. Examples of cyanine dyes include, but are not limited to, Cy2, Cy3, Cy3B, Cy3.5, Cy5, Cy5.5, Cy7, Cy7.5, sulfo-Cy3, sulfo-Cy5, and sulfo-Cy7. Examples of fluorescein dyes include, but are not limited to, fluorescein, fluorescein maleimide (FM), 5-amino-fluorescein, 6-amino-fluorescein, fluorescein isothiocyanate (FITC), fluorescein amidite (FAM), eosin, calcein, merbromin, erythrosine, NHS-fluorescein, Rose Bengal, DyLight Fluor, Oregon Green, Tokyo Green, Singapore Green, Philadelphia Green, Iodocyanine Green and the like. X can be a cyanine or a fluorescein dye. X can be fluorescein maleimide or FITC.

Representative near infrared dyes that can be used include, but are not limited to, Alexa Fluor® 680, Cy®5.5, DyLight® 680, IRDye® 680LT, Alexa Fluor® 750, Cy®7, DyLight® 750, IRDye® 750, DyLight® 800, IRDye® 800CW, Alexa Fluor® 790, CF®680, CF®680R, CF®750, CF®770, CF®790, CF®800, LS288, IR800, SP054, S0121, KODAK, IRD28, S2076, S0456, and derivatives thereof. X can be a near infrared dye. X can be 50456.

An active agent can be a chelating agent. A chelating agent can be any agent that can bind a metal or ion. A chelating agent can comprise a plurality of amines. A chelating agent can be cyclic and can contain three or more amines. A chelating agent can be selected from the group consisting of: DOTA, NOTA, NOTP, PCTA, DATA^{M}, TRAP, DFO, THP, HBED, DEDPA, TACN, TACN-TM, NODASA, NOTPME, PrP9, TACD, H₃NOKA, TACN-meHP, TACN-HP, TACN-TX, TACN-HB, TACN-TM-Bn, p-NO₂-Bn-NOTA, p-NO₂-Bn-Oxo, p-NO₂-Bn-DOTA, and p-NO₂-Bn-PCTA, X can be DOTA. X can be NOTA. X can be NOTP. X can be PCTA. X can be TACN.

X can comprise a radioisotope. X can comprise a chelating agent bound (e.g., in any suitable manner, such as through chelation) to a radioisotope. A radioisotope can be useful in the detection of a tumor or fibrotic tissue (e.g., via PET). A radioisotope can be useful in the treatment of a tumor or fibrotic tissue (e.g., radiotherapy). \X can comprise (e.g., can contain a chelating agent bound to) ^{99m}Tc, ¹¹¹In, ⁶⁷Ga, 105Rh, ¹²³I, ¹⁴⁷Nd, ¹⁵¹Pm, ¹⁵³Sm, ¹⁵⁹Gd, ¹⁶¹T, ¹⁷¹Er, ¹⁸⁶Re, ¹⁸⁸Re, or ²⁰¹Tl. X can comprise ^{99m}Tc or ¹¹¹In, or a chelated complex (e.g., NOTA, DOTA, and the like) thereof. X can comprise ^{99m}Tc. X can comprise ¹¹¹In. X can comprise ¹⁸F, ⁶⁸Ga, or a chelated complex (e.g., NOTA, DOTA, and the like) thereof. X can comprise ¹⁸F. X can comprise ⁶⁸Ga. X can be bound (e.g., in any suitable manner, such as through chelation) to a radio-imaging agent selected from the group consisting of ^{99m}Tc, ¹¹¹In, ¹⁸F, ⁶⁸Ga, ¹²⁴I, ¹²⁵I, and ¹³¹I.

X can comprise a metal or metal-chelator complex for the treatment of cancer. X can comprise arsenic, antimony, bismuth, gold, lutetium, vanadium, iron, rhodium, titanium, gallium, or platinum, or a combination of any of the aforementioned metals complexed with a chelating agent. X can comprise an arsenic-chelated complex. X can comprise a bismuth-chelated complex. X can comprise a rhodium-chelated complex. X can comprise a gallium-chelated complex. X can comprise a platinum-chelated complex. Any isotope of the aforementioned metals can be utilized in X. X can be useful in radionuclide therapy. X can comprise a radiotherapeutic agent selected from the group consisting of ³²P, ⁸⁹Sr, ⁹⁰Y, ^{117m}Sn, ¹³¹I, ¹⁵³Sm, ¹⁶⁹E, ¹⁷⁷Lu, ¹⁸⁶Re, ¹⁸⁸Re, ¹⁴⁹Tb, ²¹¹At, ²¹²Bi, ²¹³Bi, and ²²⁵Ac. X can comprise a radiotherapeutic agent ⁹⁰Y, ¹⁷⁷Lu, or ²²⁵Ac, or a chelated complex thereof (e.g., chelated by NOTA, DOTA, and the like). X can comprise ⁹⁰Y. X can comprise radiotherapeutic agent that is ¹⁷⁷Lu. X can comprise ²²⁵Ac.

Active agents can be various forms of therapeutics as well. For example, X can be an anti-cancer or anti-fibrotic drug. X can be a therapeutic agent selected from antimitotic agents. DNA alkylators, protein synthesis inhibitors, antimetabolites, and antitumor antibiotics. X can be an antimitotic agent. Non-limiting examples of antimitotic agents include paclitaxel, docetaxel, eribulin, or estramustine. X can be a DNA alkylator. By way of non-limiting example, X may be a DNA alkylator selected from cyclophosphamide, cisplatin, or carboplatin. X can be a protein synthesis inhibitor. As a non-limiting example, X can be a protein synthesis inhibitor selected from the following: rifamycin, linezolid, aminoglycosides, tetracyclines, chloramphenicol, and derivatives thereof. X can be an antimetabolite. Some examples of antimetabolites, such as can be used in X, include, but are not limited to, 5-fluorouracil, 6-mercaptopurine, capecitabine, cytarabine, thioguanine, and derivatives or analogs thereof. X can be an antitumor antibiotic. Non-limiting examples of antitumor antibiotics include tetracyclines, doxorubicin, daunorubicin, dactinomycin, and derivatives or analogs thereof.

X can comprise antibodies, antibody fragments, toxins, siRNAs, miRNAs, shRNAs, and proteolysis-targeting chimeras (PROTACs). X can comprise a small interfering RNA (“siRNA”). X can comprise a microRNA (“miRNA”). X can comprise a short hairpin RNA (“shRNA”).

X can comprise a therapeutic agent selected from inhibitors of fibroblast growth factor receptor (FGFR) isoforms, inhibitors of platelet-derived growth factor receptor (PDGFR) isoforms, inhibitors of vascular endothelial growth factor receptor (VEGFR) isoforms, inhibitors of phosphoinositide 3-kinase (PI3K) isoforms, inhibitors of Rho-associated protein kinase (ROCK), inhibitors of focal adhesion kinase (FAK) isoforms, modulators of SMAD isoforms, modulators of stimulator of interferon genes (STING) isoforms, inhibitors of toll-like receptor (TLR) isoforms (e.g., TLR7), tubulysin isoforms (e.g., tubulysin B), inhibitors of transforming growth factor beta (TGFβ) receptor, modulators of β-catenin/Wnt pathways, and inhibitors of nuclear factor kappa-light-chain-enhancer of activated B cells (NF-κB).

X can comprise a therapeutic agent selected from inhibitors of fibroblast growth factor receptor (FGFR) isoforms, inhibitors of platelet-derived growth factor receptor (PDGFR) isoforms, and inhibitors of vascular endothelial growth factor receptor (VEGFR) isoforms. X can comprise an inhibitor of FGFR. Non-limiting examples of inhibitors of FGFR include ponatinib, dovitinib, rogaratinib, or analogs thereof. X can comprise a PDGFR inhibitor. Non-limiting examples of PDGFR inhibitors include sorafenib, imatinib (and imatinib mesylate), sunitinib, ponatinib, axitlinib, nintedanib, or analogs thereof. X can comprise an inhibitor of VEGFR. Examples of inhibitors of VEGFR include, but are not limited to, regorafenib, sorafenib, and sunitinib.

X can comprise a therapeutic agent selected from inhibitors of phosphoinositide 3-kinase (PI3K) isoforms, inhibitors of ROCK, inhibitors of FAK isoforms, modulators of SMAD and/or TGF-β isoforms, and modulators of STING isoforms.

X can comprise a therapeutic agent that is an inhibitor of PI3K isoforms. X can comprise a therapeutic agent that is an inhibitor of ROCK. X can comprise a ROCK1 inhibitor. X can comprise a ROCK2 inhibitor. X can comprise a FAK inhibitor. Non-limiting examples of FAK inhibitors include defactinib, nitidine, masitinib, and conteltinib.

X can comprise an inhibitor of SMAD and/or TGF-β. By way of non-limiting example, an inhibitor of SMAD and/or TGF-β may be SRI-011381, kartogenin, pirfenidone, (E)-SIS3, or asiaticoside. X can comprise a SMAD and/or TGF-β inhibitor of Table 5, or a radical thereof.

TABLE 5
Inhibitors of SMAD and/or TGF-β.

| Com- | |
|---|---|
| pound | Structure/Description |
| X^{TGFA} | 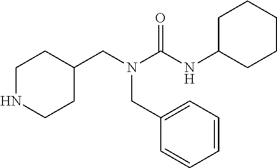 [SMILES: O=C(NC1CCCCC1)N(Cc1ccccc1)CC1CCNCC1] |
| X^{TGFB} | 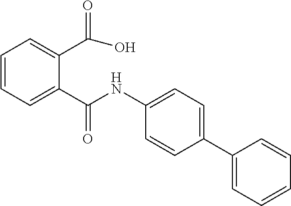 [SMILES: O=C(O)c1ccccc1C(=O)Nc1ccc(-c2ccccc2)cc1] |
| X^{TGFC} | 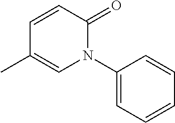 |
| X^{TGFD} | 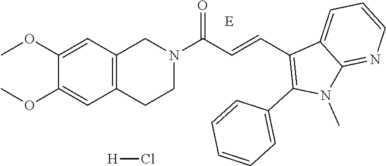 [SMILES: COc1cc2c(cc1OC)CN(C(=O)/C=C/c1c(-c3ccccc3)n(C)c3ncccc13)CC2 ; Cl] |

X can comprise an inhibitor of the STING pathway. Examples of STING pathway inhibitors include, but are not limited to, omaveloxolone (RTA 408), GSK690693, carbonyl cyanide 3-chlorophenylhydrazone, C-178, and C-176. X can comprise a STING inhibitor of Table 6, or a radical thereof.

TABLE 6
Inhibitors of the STING pathway.

| Com- | |
|---|---|
| pound | Structure/Description |
| X^{STINGA} | 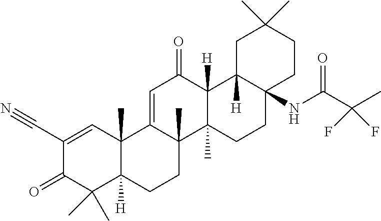 |
| X^{STINGB} | 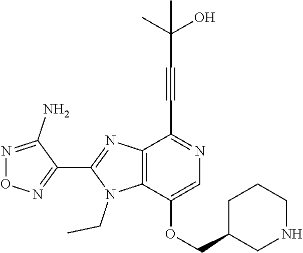 [SMILES: CCn1c(-c2nonc2N)nc2c(C#CC(C)(C)O)ncc(OC[C@H]3CCCNC3)c21] |
| X^{STINGC} | 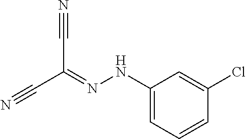 [SMILES: N#CC(C#N)=NNc1cccc(Cl)c1] |
| X^{STINGD} | 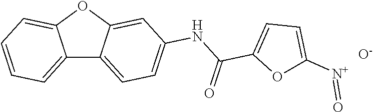 |
| X^{STINGE} | 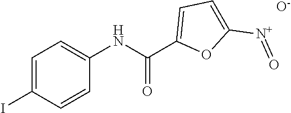 |

X can comprise a therapeutic agent selected from modulators (e.g., activators, agonists, inhibitors, antagonists) of TLR isoforms (e.g., TLR7), tubulysin isoforms (e.g., tubulysin B), inhibitors of TGFβ receptor, modulators of β-catenin/Wnt pathways, and inhibitors of NF-κB. X can comprise a TLR agonist. X can comprise an activator of TLR7. X can comprise a TLR modulator selected from Table 7, or a radical thereof.

TABLE 7
TLR agonists.

| Compound | Structure/Description |
|---|---|
| X^{TLRA} | 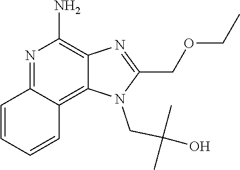 [SMILES: CCOCc1nc2c(N)nc3ccccc3c2n1CC(C)(C)O] |
| X^{TLRB} | 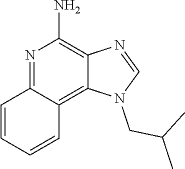 [SMILES: CC(C)Cn1cnc2c(N)nc3ccccc3c21] |
| X^{TLRC} | 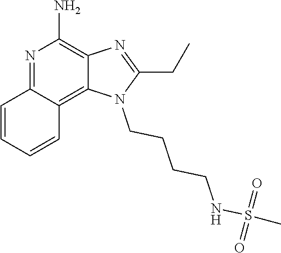 [SMILES: CCc1nc2c(N)nc3ccccc3c2n1CCCCNS(C)(=O)=O] |
| X^{TLRD} | 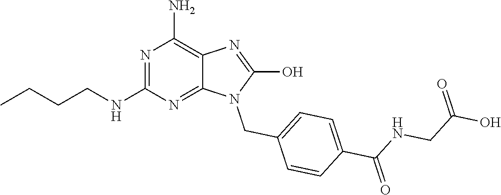 [SMILES: CCCCNc1nc(N)c2nc(O)n(Cc3ccc(C(=O)NCC(=O)O)cc3)c2n1] |
| X^{TLRE} | 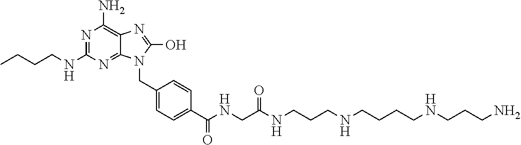 [SMILES: CCCCNc1nc(N)c2nc(O)n(Cc3ccc(C(=O)NCC(=O)NCCCNCCCCNCCCN)cc3)c2n1] |
| X^{TLRF} | 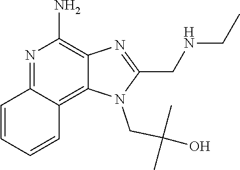 [SMILES: CCNCc1nc2c(N)nc3ccccc3c2n1CC(C)(C)O] |
| X^{TLRG} | 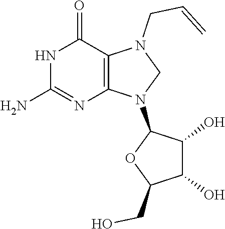 [SMILES: C=CCN1CN([C@@H]2O[C@H](CO)[C@@H](O)[C@H]2O)c2nc(N)[nH]c(=O)c21] |
| X^{TLRH} | 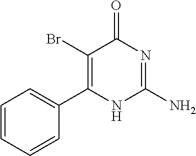 [SMILES: Nc1nc(=O)c(Br)c(-c2ccccc2)[nH]1] |
| X^{TLRI} | 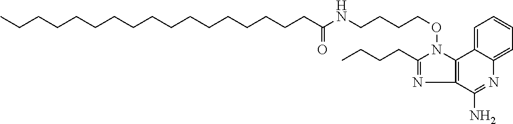 [SMILES: CCCCCCCCCCCCCCCCCC(=O)NCCCCOn1c(CCCC)nc2c(N)nc3ccccc3c21] |
| X^{TLRJ} | 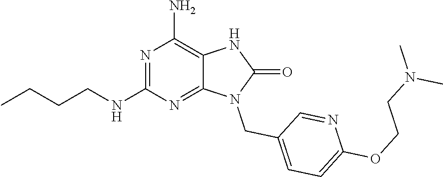 [SMILES: CCCCNc1nc(N)c2[nH]c(=O)n(Cc3ccc(OCCN(C)C)nc3)c2n1] |
| X^{TLRK} | 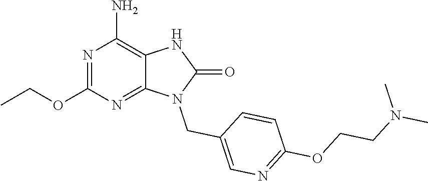 [SMILES: CCOc1nc(N)c2[nH]c(=O)n(Cc3ccc(OCCN(C)C)nc3)c2n1] |
| X^{TLRL} | 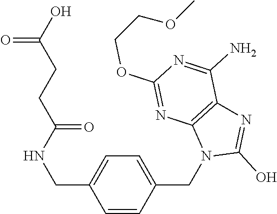 [SMILES: COCCOc1nc(N)c2nc(O)n(Cc3ccc(CNC(=O)CCC(=O)O)cc3)c2n1] |
| X^{TLRM} | 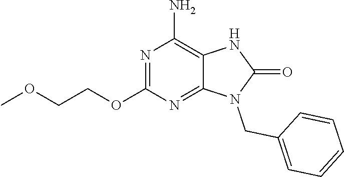 [SMILES: COCCOc1nc(N)c2[nH]c(=O)n(Cc3ccccc3)c2n1] |
| X^{TLRN} | 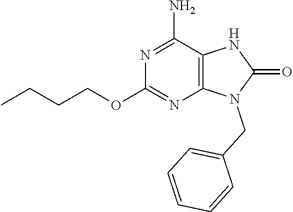 [SMILES: CCCCOc1nc(N)c2[nH]c(=O)n(Cc3ccccc3)c2n1] |
| X^{TLRO} | 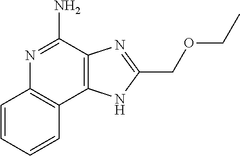 [SMILES: CCOCc1nc2c(N)nc3ccccc3c2[nH]1] |
| X^{TLRP} | 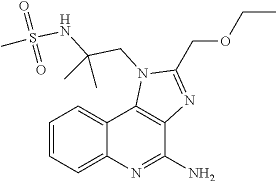 [SMILES: CCOCc1nc2c(N)nc3ccccc3c2n1CC(C)(C)NS(C)(=O)=O] |
| X^{TLRQ} | See, e.g., Lipanov et al., The structure of poly(dA): poly (dT) in a condensed state and in |
| | solution Nucleic Acids Research, 15(14): 5833-5844 (1987). |
| X^{TLRR} | 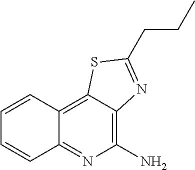 [SMILES: CCCc1nc2c(N)nc3ccccc3c2s1] |
| X^{TLRS} | 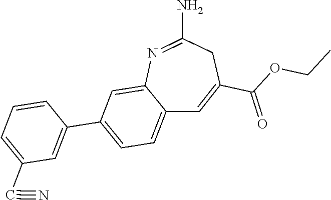 [SMILES: CCOC(=O)C1=Cc2ccc(-c3cccc(C#N)c3)cc2N=C(N)C1] |
| X^{TLRT} | 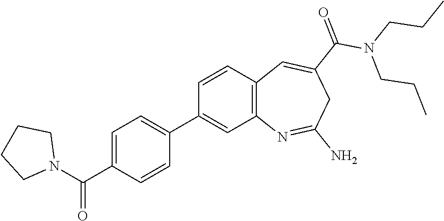 [SMILES: CCCN(CCC)C(=O)C1=Cc2ccc(-c3ccc(C(=O)N4CCCC4)cc3)cc2N=C(N)C1] |
| X^{TLRU} | Short synthetic single-stranded DNA molecules containing unmethylated CpG dinucleotides |
| | in particular sequence contexts (CpG motifs) (CpG ODN) |
| X^{TLRV} | Synthetic oligonucleotide containing unmethylated CpG dinucleotides with potential |
| | immunopotentiating activity (IMO 2005) |
| X^{TLRW} | Short, synthetic, unmethylated CpG oligodeoxynucleotide (CpG ODN) with immunot- |
| | stimulatory activity (1018-ISS) |
| X^{TLRX} | Comprises a strand of inosine poly(I) homopolymer annealed to a strand of cytidine |
| | (poly(I:C)) |
| X^{TLRY} | Poly(C)homopolymer |
| X^{TLRZ} | 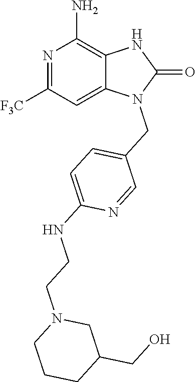 [SMILES: Nc1nc(C(F)(F)F)cc2c1[nH]c(=O)n2Cc1ccc(NCCN2CCCC(CO)C2)nc1] |

X can comprise a tubulysin B. X can comprise a radical of tubulysin B, or a derivative thereof. X can comprise an inhibitor of the Wnt/β-catenin signaling pathway, or a radical thereof. Non-limiting examples of Wnt/β-catenin inhibitors include IWR-1, IWP-2, pyrvinium pamoate, salinomycin, adavivint, and wogonin.

X can comprise an inhibitor of NF-κB, or a radical thereof. X can comprise a structure of Table 8, or a radical thereof.

TABLE 8
Inhibitors of NF-κB.

| Compound | Structure |
|---|---|
| X^{NFKBA} | 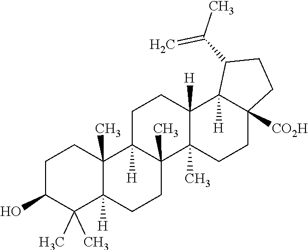 |
| X^{NFKBB} | 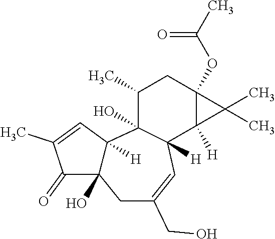 |
| X^{NFKBC} | 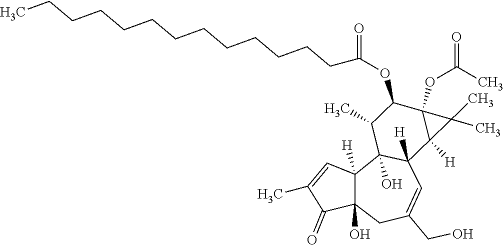 |
| X^{NFKBD} | 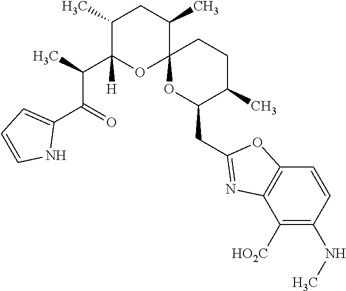 [SMILES: CNc1ccc2oc(C[C@H]3O[C@@]4(CC[C@H]3C)O[C@H]([C@H](C)C(=O)c3ccc[nH]3)[C@H](C)C[C@H]4C)nc2c1C(=O)O] |
| X^{NFKBE} | 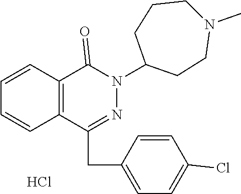 [SMILES: CN1CCCC(n2nc(Cc3ccc(Cl)cc3)c3ccccc3c2=O)CC1 ; Cl] |
| X^{NFKBF} | 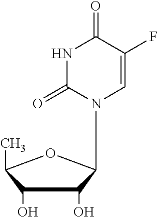 |
| X^{NFKBG} | 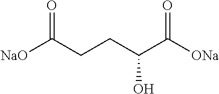 |

A compound or compound of the disclosure can have a structure of Table 9.

TABLE 9
Example compounds

| Com- | | | | | |
|---|---|---|---|---|---|
| pound | Q | L^{Q} | Y | L^{X} | X |
| 1a | Q^{A} | L^{QC}, n = 6 | Y^{F}, m = 2 | L^{XI}, n = 1 | Rhodamine |
| 1b | Q^{A} | L^{QC}, n = 12 | Y^{F}, m = 2 | L^{XI}, n = 1 | Rhodamine |
| 2a | Q^{A} | L^{QC}, n = 6 | Y^{F}, m = 2 | L^{XI}, n = 1 | S0456 |
| 2b | Q^{A} | L^{QC}, n = 12 | Y^{F}, m = 2 | L^{XI}, n = 1 | S0456 |
| 3a | Q^{A} | L^{QC}, n = 6 | Y^{E}, m = 2 | L^{XI}, n = 1 | Rhodamine |
| 3b | Q^{A} | L^{QC}, n = 12 | Y^{E}, m = 2 | L^{XI}, n = 1 | Rhodamine |
| 4a | Q^{A} | L^{QC}, n = 6 | Y^{E}, m = 2 | L^{XI}, n = 1 | S0456 |
| 4b | Q^{A} | L^{QC}, n = 12 | Y^{E}, m = 2 | L^{XI}, n = 1 | S0456 |
| 5a | Q^{A} | L^{QC}, n = 6 | Y^{D} m = 2 | L^{XI}, n = 1 | Rhodamine |
| 5b | Q^{A} | L^{QC}, n = 12 | Y^{D}, m = 2 | L^{XI}, n = 1 | Rhodamine |
| 5c | Q^{D} | L^{QC}, n = 6 | Y^{D}, m = 2 | L^{XI}, n = 1 | Rhodamine |
| 5d | Q^{D} | L^{QC}, n = 12 | Y^{D}, m = 2 | L^{XI}, n = 1 | Rhodamine |
| 6a | Q^{A} | L^{QC}, n = 6 | Y^{D}, m = 2 | L^{XI}, n = 1 | S0456 |
| 6b | Q^{A} | L^{QC}, n = 12 | Y^{D}, m = 2 | L^{XI}, n = 1 | S0456 |
| 6c | Q^{D} | L^{QC}, n = 6 | Y^{D}, m = 2 | L^{XI}, n = 1 | S0456 |
| 6d | Q^{D} | L^{QC}, n = 12 | Y^{D}, m = 2 | L^{XI}, n = 1 | S0456 |
| 7a | Q^{A} | L^{QC}, n = 6 | Y^{D}, m = 2 | L^{XI}, n = 1 | fluorescein |
| 7b | Q^{A} | L^{QC}, n = 12 | Y^{D}, m = 2 | L^{XI}, n = 1 | fluorescein |
| 7c | Q^{D} | L^{QC}, n = 6 | Y^{D}, m = 2 | L^{XI}, n = 1 | fluorescein |
| 7d | Q^{D} | L^{QC}, n = 12 | Y^{D}, m = 2 | L^{XI}, n = 1 | fluorescein |
| 7a | Q^{A} | L^{QC}, n = 6 | Y^{D}, m = 2 | L^{XI}, n = 1 | DOTA |
| 7b | Q^{A} | L^{QC}, n = 12 | Y^{D}, m = 2 | L^{XI}, n = 1 | DOTA |
| 7c | Q^{D} | L^{QC}, n = 6 | Y^{D}, m = 2 | L^{XI}, n = 1 | DOTA |
| 7d | Q^{D} | L^{QC}, n = 12 | Y^{D}, m = 2 | L^{XI}, n = 1 | DOTA |
| 7e | Q^{A} | L^{QC}, n = 18 | Y^{D}, m = 2 | L^{XI}, n = 1 | DOTA |
| 7f | Q^{D} | L^{QC}, n = 18 | Y^{D}, m = 2 | L^{XI}, n = 1 | DOTA |
| 8a | Q^{A} | L^{QC}, n = 6 | Y^{D}, m = 2 | L^{XI}, n = 1 | NOTA |
| 8b | Q^{A} | L^{QC}, n = 12 | Y^{D,} m = 2 | L^{XI}, n = 1 | NOTA |
| 8c | Q^{D} | L^{QC}, n = 6 | Y^{D}, m = 2 | L^{XI}, n = 1 | NOTA |
| 8d | Q^{D} | L^{QC}, n = 12 | Y^{D}, m = 2 | L^{XI}, n = 1 | NOTA |
| 9a | Q^{A} | L^{QC}, n = 6 | Y^{D}, m = 2 | L^{XH}, n = 5 | DOTA |
| | | | | W = glucosamine | |
| 9b | Q^{A} | L^{QC}, n = 12 | Y^{D}, m = 2 | L^{XH}, n = 5 | DOTA |
| | | | | W = glucosamine | |
| 10a | Q^{A} | L^{QC}, n = 12 | Y^{D}, m = 2 | L^{XH}, n = 5 | DOTA |
| | | | | W = PEGn′ | |
| | | | | n′ = 6 | |
| 10b | Q^{A} | L^{QC}, n = 12 | Y^{D}, m = 2 | L^{XI}, n = 5 | DOTA |
| | | | | W = PEGn′ | |
| | | | | n′ = 12 | |

A compound of the disclosure can have one of the following structures:

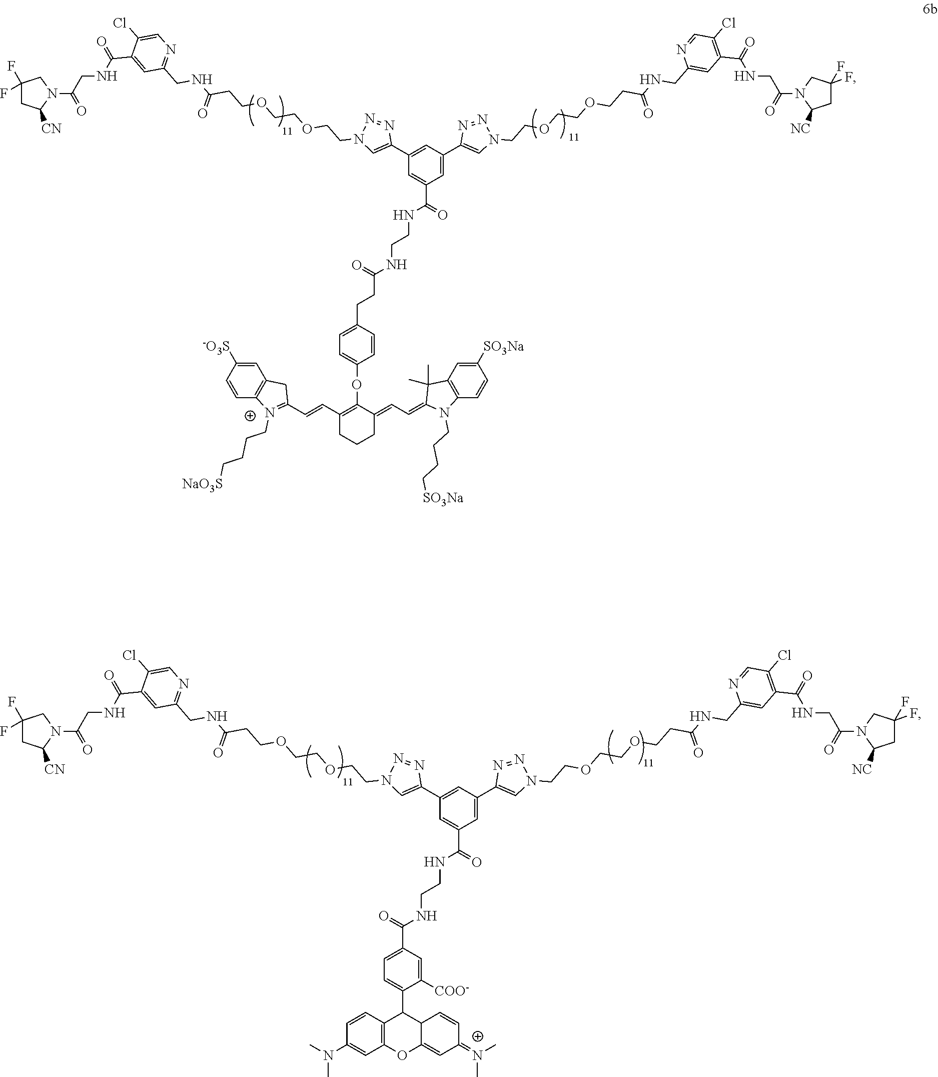

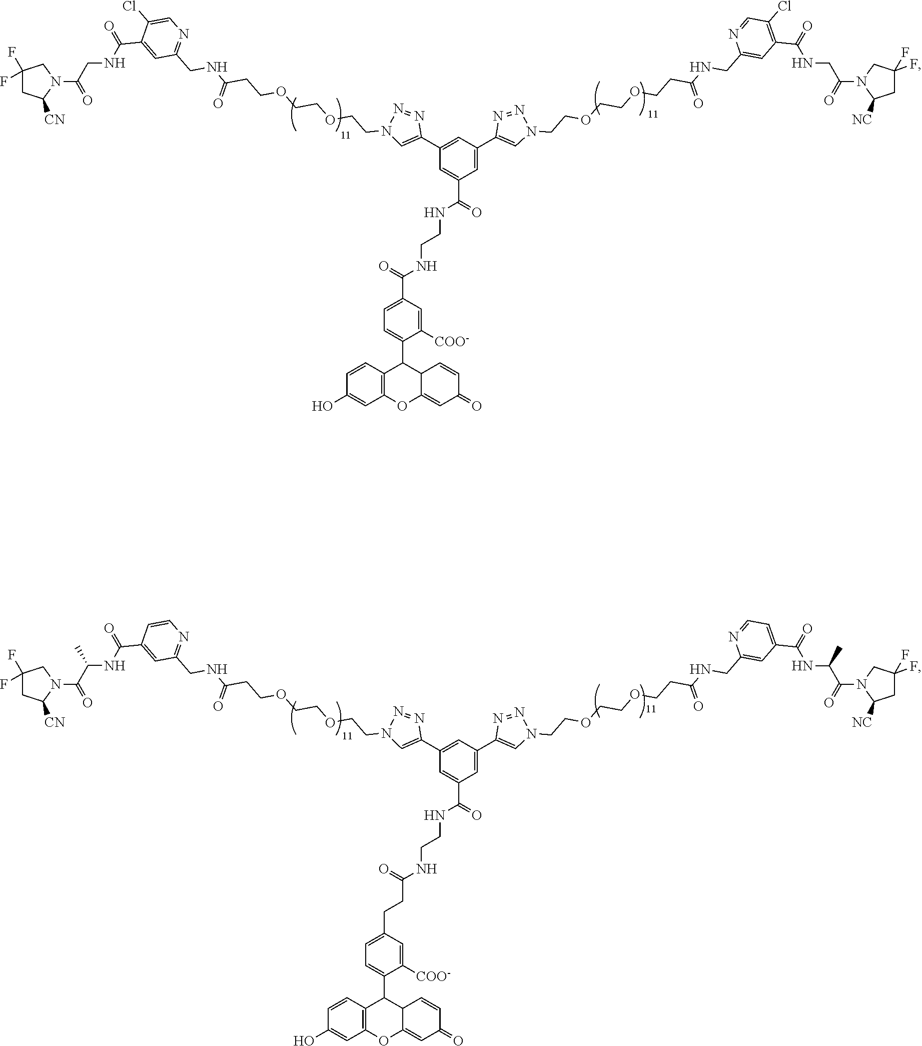

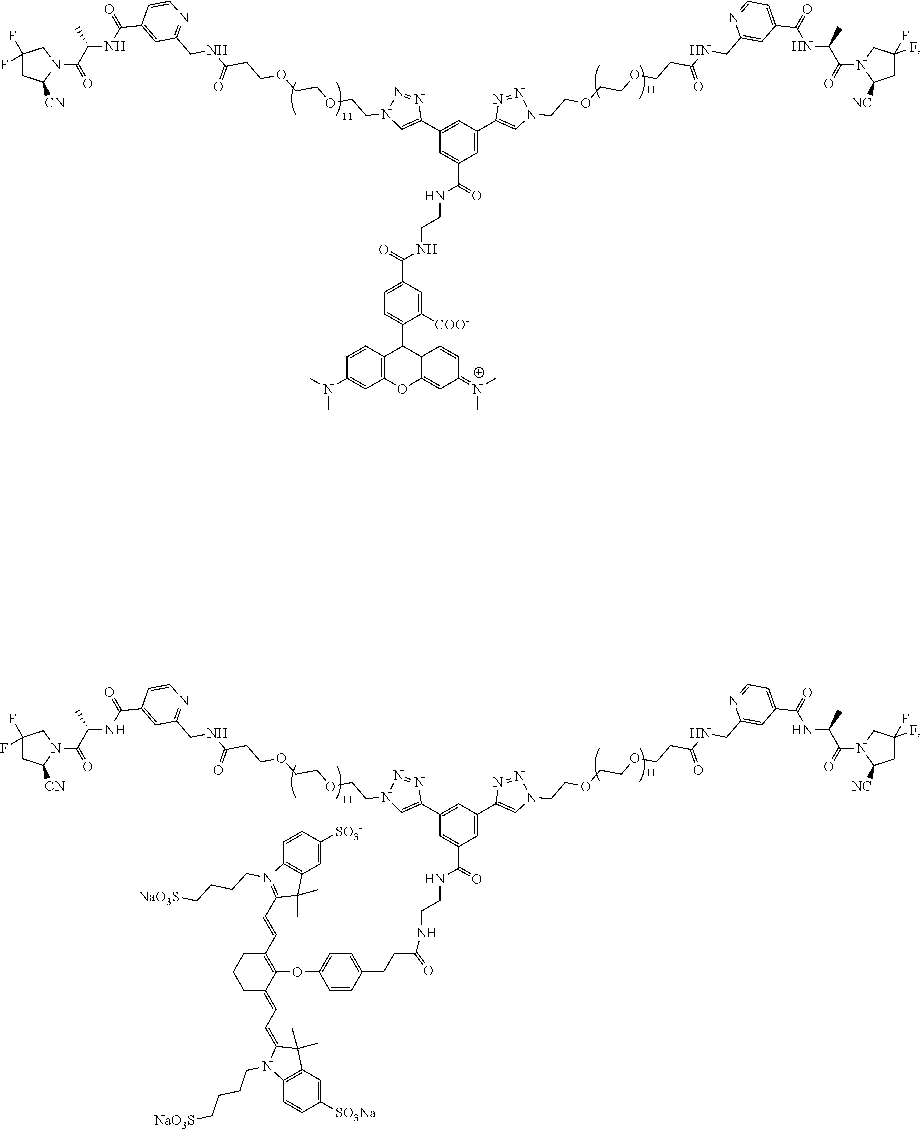

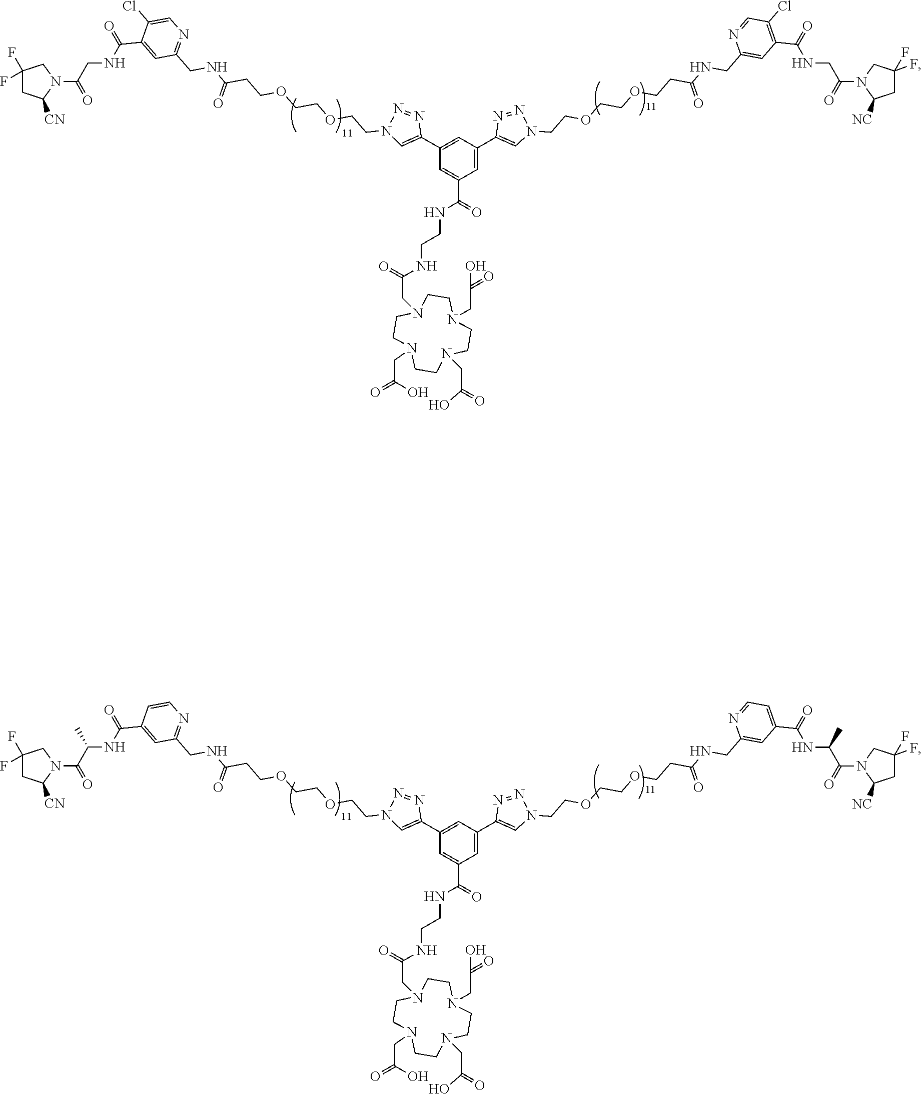

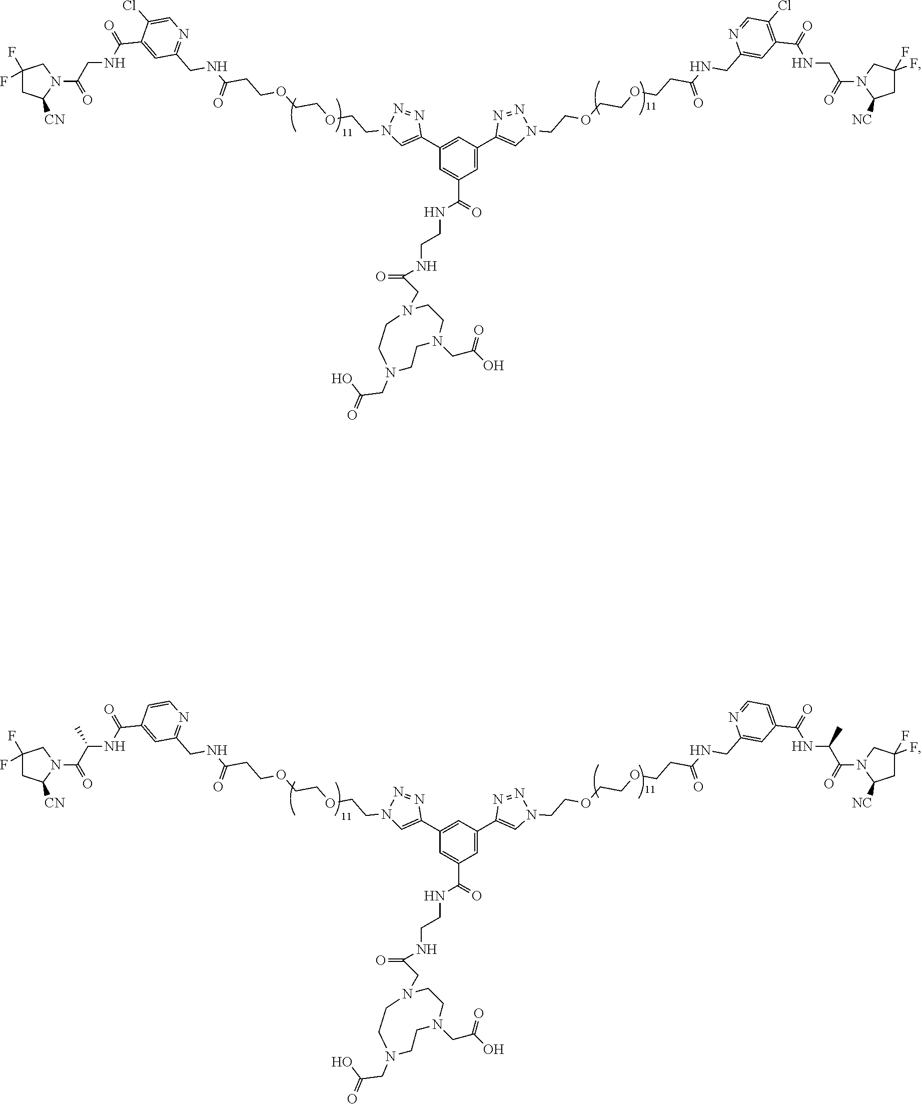

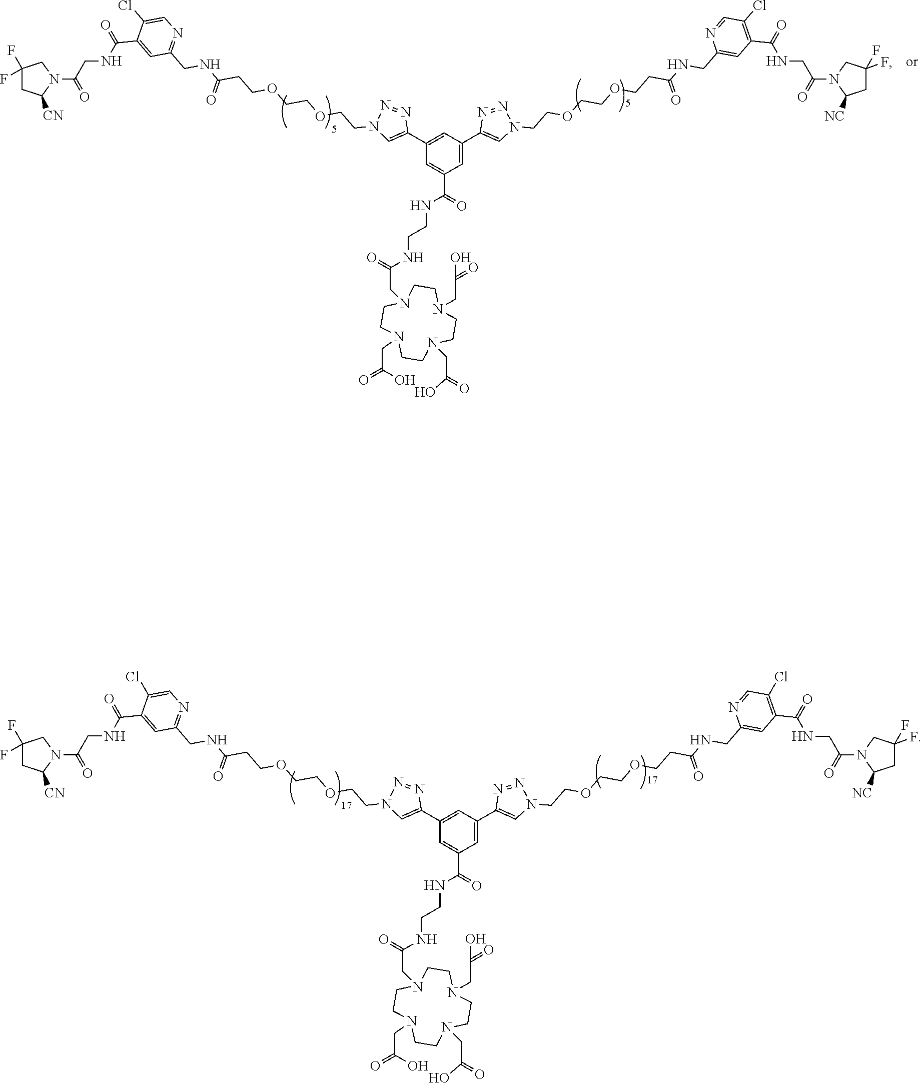

A compound that has the following structure:

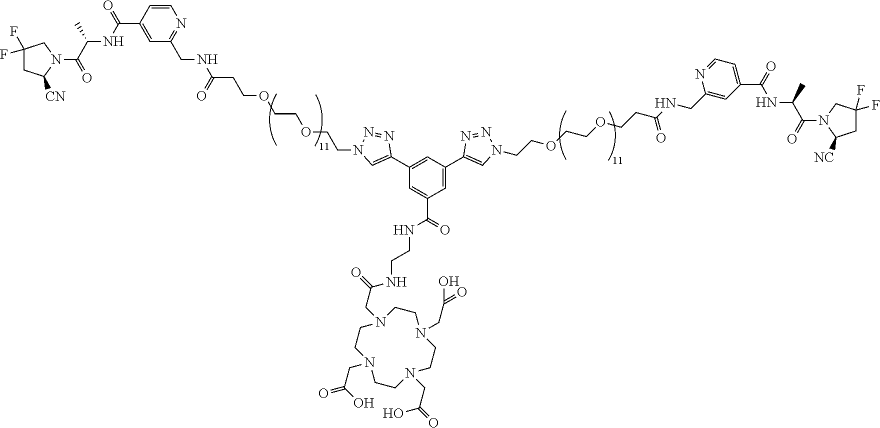

A compound that has the following structure:

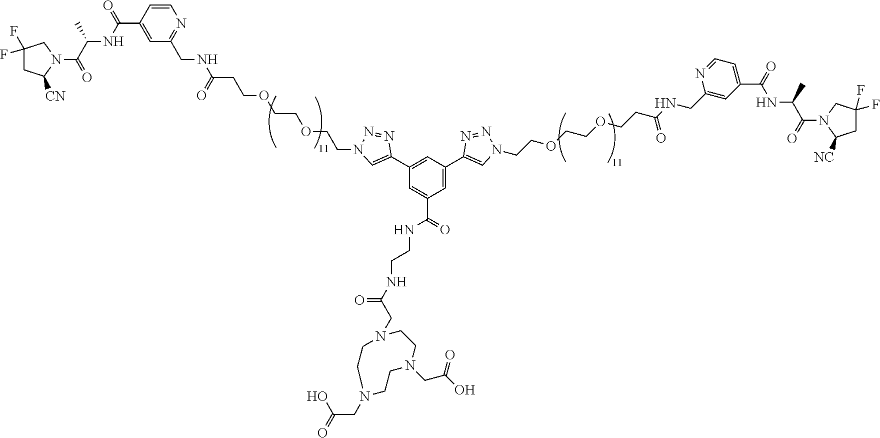

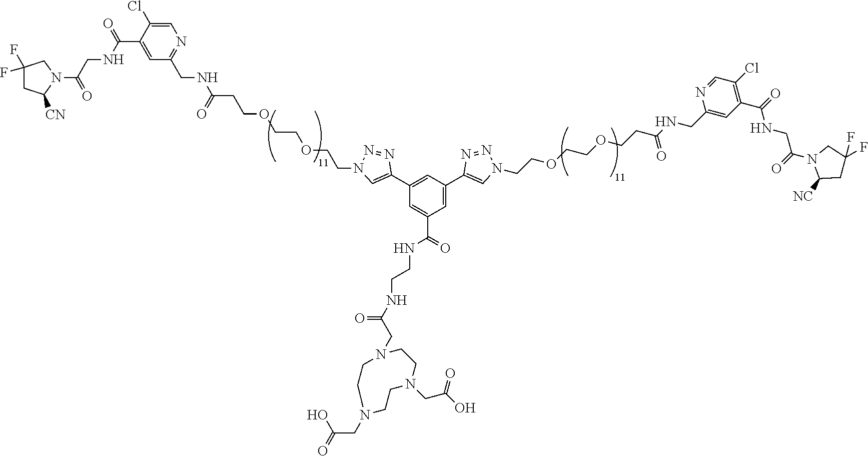

### Methods of Treatment

A compound can be effective in detecting or treating a disease. For example, a compound with a chelating agent X bound (e.g., in any suitable manner, such as through chelation) to a gamma-emitting radionuclide can be effective in detecting a tumor. Similarly, a compound with a chelating agent X bound to a strontium-89 or radium-223 metal can be effective in treating a tumor. A compound can be effective at treating or detecting non-tumor diseases, disorders, or conditions as well. A compound is effective in treating fibrosis, idiopathic pulmonary fibrosis (IPF), chronic kidney disease, skin fibrosis, fibrotic liver disease, cardiac fibrosis, cancer, melanoma, colorectal cancer, pancreatic cancer, breast cancer, sarcoma, esophageal cancer. Chagas disease cardiomyopathy (CCC), lung cancer, head and neck cancer, cancer of unknown primary (CUP), medullary thyroid cancer (MTC), thymus cancer, neuroendocrine tumors (NET), small-intestine cancer, prostate cancer, or a combination thereof. A compound is effective in treating fibrosis, idiopathic pulmonary fibrosis (1PF), chronic kidney disease, skin fibrosis, fibrotic liver disease, cardiac fibrosis, or a combination thereof. A compound is effective in treating fibrosis. A compound is effective in treating idiopathic pulmonary fibrosis (IPF), skin fibrosis, fibrotic liver disease, cardiac fibrosis, or a combination thereof. A compound is effective in treating cancer, melanoma, colorectal cancer, pancreatic cancer, breast cancer, sarcoma, esophageal cancer, CCC, lung cancer, head and neck cancer, CUP, MTC, thymus cancer, NET, small-intestine cancer, prostate cancer, or a combination thereof. A compound is effective in treating cancer. A compound is effective in treating melanoma. A compound is effective in treating colorectal cancer. A compound is effective in treating pancreatic cancer. A compound is effective in treating breast cancer. A compound is effective in treating esophageal cancer. A compound is effective in treating head and neck cancer. A compound is effective in treating lung cancer. A compound is effective in treating small intestine cancer. A compound is effective in treating prostate cancer. A compound is effective in treating CCC. A compound is effective in treating CUP. A compound is effective in treating MTC. A compound is effective in treating NET. A compound is effective in treating sarcoma. A compound is effective in treating a tumor. A compound is effective in treating a tumor associated with CAFs. A compound is effective in treating a tumor overexpressing FAP. A compound is effective in treating a disease associated with CAFs. A compound is effective in treating a disease associated with overexpression of FAP. A compound is effective in treating a disease characterized by overexpression, hyperproliferation, or otherwise aberrant function of fibroblasts. A compound is effective in treating a fibrotic disease associated with overexpression of FAP in myofibroblasts or activated fibroblasts.

A compound is effective in treating a disease or disorder (e.g., a cancer or fibrotic condition) that was previously refractory or resistant to treatment.

In some embodiments, a compound can be internalized. A compound binds two chains of a FAP dimer. Dimerization of FAP can facilitate catalytic activity, and anti-FAP antibodies but not monovalent Fabs internalize in FAP-positive cells after binding to FAP. In certain instances herein, a ligand-targeted agent containing two or more FAP-binding ligands (e.g., with desired spacer length and desired physiochemical properties) can be used to induce or enhance internalization of the drugs and/or imaging agents attached thereto. Binding a FAP dimer can facilitate internalization of the compound. A compound is more effective following internalization. A compound can be retained for 1, 2, 3, 4, 6, 8, 12, 18, 24, 36, 48 h or more. A compound can be detectable for 1, 2, 3, 4, 6, 8, 12, 18, 24, 36, 48 h or more. A dual-FAP compound can be eliminated or excreted more slowly than a mono-FAP or FAP antibody. A multivalent compound can be used in diagnosing a disease. The disease can be cancer. The disease can be a fibrotic disease or disorder. A multivalent compound can be used to identify the source of a disease (e.g., a cancer). A compound can deliver a radioactive payload to the interior of a cell (e.g., a fibroblast or CAF). A compound can deliver a near infrared dye to the interior of a cell (e.g., a fibroblast or CAF). A compound can deliver a fluorescent dye to the interior of a cell (e.g., a fibroblast or CAF). A compound can deliver an anticancer therapeutic to the interior of a cell (e.g., a fibroblast or CAF).

A method of providing an active agent in proximity to a CAF or FAP-expressing cell is also provided. The method comprises administering a compound to a CAF or a cell that expresses FAP. The compound is retained within the CAF or the FAP-expressing cell for at least 24 hours.

Another method of providing an active agent in proximity to a CAF or FAP-expressing cell is also provided. The method comprises administering a compound to a subject comprising, or suspected of comprising, a plurality of CAFs or FAP-expressing cells. The compound can be retained within the CAF or FAP-expressing cells for at least 24 hours. “Within the cell” can be that the compound can be retained inside the cell or on the membrane of the cell.

A method of detecting a tumor or fibrotic tissue in a subject is also provided. The method comprises (i) administering a compound to a subject suspected of having a tumor or fibrotic tissue, (ii) detecting the compound within the subject (e.g., optically or radiometrically), and (iii) identifying the tumor or fibrotic tissue in the subject based on the localization of the compound.

Also provided is a method for the treatment of a tumor or fibrotic tissue in a subject. The method comprises administering to the individual a therapeutically effective amount of an above-described compound.

### Pharmaceutical Compositions, Routes of Administration, and Dosing

Pharmaceutical compositions are also provided. In an embodiment, the pharmaceutical composition comprises a compound and a pharmaceutically acceptable carrier. In another embodiment, the pharmaceutical composition comprises a plurality of compounds and a pharmaceutically acceptable carrier. In yet another embodiment, the pharmaceutical composition comprises a prodrug of a compound, alone or in further combination with one or more other compounds described herein, or prodrugs thereof, and a pharmaceutically acceptable carrier.

A pharmaceutical composition can further comprise at least one additional pharmaceutically active agent other than a compound. The at least one additional pharmaceutically active agent can be, for example, an agent useful in the treatment of ischemia-reperfusion injury.

Pharmaceutical compositions can be prepared by combining one or more compounds with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound being administered, the size of the subject, or the severity of the disease or condition. One of ordinary skill in the art can empirically determine the effective amount of a particular compound and/or other therapeutic agent without necessitating undue experimentation. A maximum dose can be used, that is, the highest safe dose according to some medical judgment. Multiple doses per day are contemplated to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of a compound are, for human subjects, from about 0.01 milligrams/kg per day to 1000 milligrams/kg per day. Oral doses in the range of 0.5 to 50 milligrams/kg, in one or more administrations per day, can yield therapeutic results. Dosage can be adjusted appropriately to achieve desired drug levels, local or systemic, depending upon the mode of administration. For example, intravenous administration can vary from one order to several orders of magnitude lower dose per day. In the event that the response in a subject is insufficient at such doses, even higher doses (or effective higher doses by a different, more localized delivery route) can be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods are well-known in the art and well within the capabilities of the ordinarily skilled artisan.

For clinical use, any compound can be administered in an amount equal or equivalent to 0.2-2.000 milligram (mg) of compound per kilogram (kg) of body weight of the subject per day. The compounds can be administered in a dose equal or equivalent to 2-2,000 mg of conjugate per kg body weight of the subject per day. The compounds can be administered in a dose equal or equivalent to 20-2,000 mg of conjugate per kg body weight of the subject per day. The compounds can be administered in a dose equal or equivalent to 50-2,000 mg of conjugate per kg body weight of the subject per day. The compounds can be administered in a dose equal or equivalent to 100-2,000 mg of compound per kg body weight of the subject per day. The compounds can be administered in a dose equal or equivalent to 200-2,000 mg of conjugate per kg body weight of the subject per day. Where a precursor or prodrug of the compounds is to be administered rather than the compound, itself, it is administered in an amount that is equivalent to, i.e., sufficient to deliver, the above-stated amounts of the compounds.

The formulations of the compounds can be administered to human subjects in therapeutically effective amounts. Typical dose ranges are from about 0.01 microgram/kg to about 2 mg/kg of body weight per day. The dosage of drug to be administered is likely to depend on such variables as the type and extent of the disorder, the overall health status of the particular subject, the specific conjugate being administered, the excipients used to formulate the compound, and its route of administration. Routine experiments may be used to optimize the dose and dosing frequency for any particular compound.

The compounds can be administered at a concentration in the range from about 0.001 microgram/kg to greater than about 500 mg/kg. For example, the concentration can be 0.001 microgram/kg, 0.01 microgram/kg, 0.05 microgram/kg, 0.1 microgram/kg, 0.5 microgram/kg, 1.0 microgram/kg, 10.0 microgram/kg, 50.0 microgram/kg, 100.0 microgram/kg, 500 microgram/kg, 1.0 mg/kg, 5.0 mg/kg, 10.0 mg/kg, 15.0 mg/kg, 20.0 mg/kg, 25.0 mg/kg, 30.0 mg/kg, 35.0 mg/kg, 40.0 mg/kg, 45.0 mg/kg, 50.0 mg/kg, 60.0 mg/kg, 70.0 mg/kg, 80.0 mg/kg, 90.0 mg/kg, 100.0 mg/kg, 150.0 mg/kg, 200.0 mg/kg, 250.0 mg/kg, 300.0 mg/kg, 350.0 mg/kg, 400.0 mg/kg, 450.0 mg/kg, to greater than about 500.0 mg/kg or any incremental value thereof. It is to be understood that all values and ranges between these values and ranges are meant to be encompassed.

The compounds can be administered at a dosage in the range from about 0.2 milligram/kg/day to greater than about 100 mg/kg/day. For example, the dosage may be 0.2 mg/kg/day to 100 mg/kg/day, 0.2 mg/kg/day to 50 mg/kg/day, 0.2 mg/kg/day to 25 mg/kg/day, 0.2 mg/kg/day to 10 mg/kg/day, 0.2 mg/kg/day to 7.5 mg/kg/day, 0.2 mg/kg/day to 5 mg/kg/day, 0.25 mg/kg/day to 100 mg/kg/day, 0.25 mg/kg/day to 50 mg/kg/day, 0.25 mg/kg/day to 25 mg/kg/day, 0.25 mg/kg/day to 10 mg/kg/day, 0.25 mg/kg/day to 7.5 mg/kg/day, 0.25 mg/kg/day to 5 mg/kg/day, 0.5 mg/kg/day to 50 mg/kg/day, 0.5 mg/kg/day to 25 mg/kg/day, 0.5 mg/kg/day to 20 mg/kg/day, 0.5 mg/kg/day to 15 mg/kg/day, 0.5 mg/kg/day to 10 mg/kg/day, 0.5 mg/kg/day to 7.5 mg/kg/day, 0.5 mg/kg/day to 5 mg/kg/day, 0.75 mg/kg/day to 50 mg/kg/day, 0.75 mg/kg/day to 25 mg/kg/day, 0.75 mg/kg/day to 20 mg/kg/day, 0.75 mg/kg/day to 15 mg/kg/day, 0.75 mg/kg/day to 10 mg/kg/day, 0.75 mg/kg/day to 7.5 mg/kg/day, 0.75 mg/kg/day to 5 mg/kg/day, 1.0 mg/kg/day to 50 mg/kg/day, 1.0 mg/kg/day to 25 mg/kg/day, 1.0 mg/kg/day to 20 mg/kg/day, 1.0 mg/kg/day to 15 mg/kg/day, 1.0 mg/kg/day to 10 mg/kg/day, 1.0 mg/kg/day to 7.5 mg/kg/day, 1.0 mg/kg/day to 5 mg/kg/day, 2 mg/kg/day to 50 mg/kg/day, 2 mg/kg/day to 25 mg/kg/day, 2 mg/kg/day to 20 mg/kg/day, 2 mg/kg/day to 15 mg/kg/day, 2 mg/kg/day to 10 mg/kg/day, 2 mg/kg/day to 7.5 mg/kg/day, or 2 mg/kg/day to 5 mg/kg/day.

The compounds can be administered at a dosage in the range from about 0.25 milligram/kg/day to about 25 mg/kg/day. For example, the dosage may be 0.25 mg/kg/day, 0.5 mg/kg/day, 0.75 mg/kg/day, 1.0 mg/kg/day, 1.25 mg/kg/day, 1.5 mg/kg/day, 1.75 mg/kg/day, 2.0 mg/kg/day, 2.25 mg/kg/day, 2.5 mg/kg/day, 2.75 mg/kg/day, 3.0 mg/kg/day, 3.25 mg/kg/day, 3.5 mg/kg/day, 3.75 mg/kg/day, 4.0 mg/kg/day, 4.25 mg/kg/day, 4.5 mg/kg/day, 4.75 mg/kg/day, 5 mg/kg/day, 5.5 mg/kg/day, 6.0 mg/kg/day, 6.5 mg/kg/day, 7.0 mg/kg/day, 7.5 mg/kg/day, 8.0 mg/kg/day, 8.5 mg/kg/day, 9.0 mg/kg/day, 9.5 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 21 mg/kg/day, 22 mg/kg/day, 23 mg/kg/day, 24 mg/kg/day, 25 mg/kg/day, 26 mg/kg/day, 27 mg/kg/day, 28 mg/kg/day, 29 mg/kg/day, 30 mg/kg/day, 31 mg/kg/day, 32 mg/kg/day, 33 mg/kg/day, 34 mg/kg/day, 35 mg/kg/day, 36 mg/kg/day, 37 mg/kg/day, 38 mg/kg/day, 39 mg/kg/day, 40 mg/kg/day, 41 mg/kg/day, 42 mg/kg/day, 43 mg/kg/day, 44 mg/kg/day, 45 mg/kg/day, 46 mg/kg/day, 47 mg/kg/day, 48 mg/kg/day, 49 mg/kg/day, or 50 mg/kg/day.

The compounds can be administered in concentrations that range from 0.01 micromolar to greater than or equal to 500 micromolar. For example, the dose can be 0.01 micromolar, 0.02 micromolar, 0.05 micromolar, 0.1 micromolar, 0.15 micromolar, 0.2 micromolar, 0.5 micromolar, 0.7 micromolar, 1.0 micromolar, 3.0 micromolar, 5.0 micromolar, 7.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 60.0 micromolar, 70.0 micromolar, 80.0 micromolar, 90.0 micromolar, 100.0 micromolar, 150.0 micromolar, 200.0 micromolar, 250.0 micromolar, 300.0 micromolar, 350.0 micromolar, 400.0 micromolar, 450.0 micromolar, to greater than about 500.0 micromolar or any incremental value thereof. It is to be understood that all values and ranges between these values and ranges are meant to be encompassed.

The compounds can be administered at concentrations that range from 0.10 microgram/mL to 500.0 microgram/mL. For example, the concentration can be 0.10 microgram/mL, 0.50 microgram/mL, 1 microgram/mL, 2.0 microgram/mL, 5.0 microgram/mL, 10.0 microgram/mL, 20 microgram/mL, 25 microgram/mL, 30 microgram/mL, 35 microgram/mL, 40 microgram/mL, 45 microgram/mL, 50 microgram/mL, 60.0 microgram/mL, 70.0 microgram/mL, 80.0 microgram/mL, 90.0 microgram/mL, 100.0 microgram/mL, 150.0 microgram/mL, 200.0 microgram/mL, 250.0 g/mL, 250.0 micro gram/mL, 300.0 microgram/mL, 350.0 microgram/mL, 400.0 microgram/mL, 450.0 microgram/mL, to greater than about 500.0 microgram/mL or any incremental value thereof. It is to be understood that all values and ranges between these values and ranges are meant to be encompassed.

The formulations can be administered in pharmaceutically acceptable solutions, which can routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include, but are not limited to, intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well-known in the art. Such carriers enable the compounds to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose (MC), hydroxypropylmethyl-cellulose (HPMC), sodium carboxymethylcellulose, and/or polyvinylpyrrolidone (PVP). If desired, disintegrating agents can be added, such as the cross-linked PVP, agar, or alginic acid or a salt thereof such as sodium alginate. Optionally the oral formulations can also be formulated in saline or buffers, e.g., EDTA for neutralizing internal acid conditions or may be administered without any carriers.

Also contemplated are oral dosage forms of the compounds. The compounds can be chemically modified so that oral delivery of the derivative is efficacious. Generally, the chemical modification contemplated is the attachment of at least one moiety to the compound itself, where said moiety permits (a) inhibition of acid hydrolysis; and (b) uptake into the blood stream from the stomach or intestine. Also desired is the increase in overall stability of the compounds and increase in circulation time in the body. Examples of such moieties include PEG, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”. In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, PEG moieties are suitable.

The location of release of a compound can be the stomach, the small intestine (the duodenum, the jejunum, or the ileum), or the large intestine. One skilled in the art has available formulations which will not dissolve in the stomach yet will release the material in the duodenum or elsewhere in the intestine. The release can avoid the deleterious effects of the stomach environment, either by protection of the compound or by release of the compound beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings can be used as mixed films.

A coating or mixture of coatings can also be used on tablets, which are not intended to be protected from the stomach. These coatings can include sugar coatings, or coatings which make the tablet easier to swallow. Capsules can consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic agent can be included in the formulation as fine multi-particulates in the form of granules or pellets of particle size about 1 mm. The formulation of the material for capsule administration could also be as a powder, lightly compressed plugs or even as tablets. The therapeutic formulation can also be prepared by compression.

Colorants and flavoring agents can be included. For example, the compound can be formulated (such as by liposome or microsphere encapsulation) and then further contained within an edible product, such as a refrigerated beverage containing colorants and flavoring agents.

One can dilute or increase the volume of the therapeutic agent with an inert material. These diluents could include carbohydrates, especially mannitol, a-lactose, anhydrous lactose, cellulose, sucrose, modified dextrans and starch. Certain inorganic salts also can be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic agent into a solid dosage form. Materials used as disintegrates include, but are not limited to, starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite all can be used. Another form of the disintegrants are the insoluble cationic exchange resins. Powdered gums can be used as disintegrants and as binders and these can include powdered gums such as agar. Karaya or tragacanth. Alginic acid and its sodium salt are also useful as disintegrants.

Binders can be used to hold the therapeutic agent together to form a hard tablet and include materials from natural products such as acacia, tragacanth, starch and gelatin. Others include MC, ethyl cellulose (EC) and carboxymethyl cellulose (CMC). PVP and HPMC could both be used in alcoholic solutions to granulate the therapeutic agent.

An anti-frictional agent can be included in the formulation of the therapeutic agent to prevent sticking during the formulation process. Lubricants can be used as a layer between the therapeutic agent and the die wall, and these can include, but are not limited to, stearic acid including its magnesium and calcium salts, polytetrafluoroethylene (PTFE), liquid paraffin, vegetable oils and waxes. Soluble lubricants also can be used, such as sodium lauryl sulfate, magnesium lauryl sulfate, PEG of various molecular weights, and Carbowax 4000 and 6000.

Glidants that might improve the flow properties of the drug during formulation and to aid rearrangement during compression can be added. The glidants can include starch, talc, pyrogenic silica and hydrated silicoaluminate.

To aid dissolution of the therapeutic agent into the aqueous environment a surfactant can be added as a wetting agent. Surfactants can include anionic detergents such as sodium lauryl sulfate, dioctyl sodium sulfosuccinate and dioctyl sodium sulfonate. Cationic detergents, which can be used, include benzalkonium chloride and benzethonium chloride. Potential non-ionic detergents that can be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants can be present in the formulation of the compound or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid PEG. In addition, stabilizers can be added. Microspheres formulated for oral administration can also be used. Such microspheres have been well-defined in the art. All formulations for oral administration should be in dosages suitable for such administration.

For buccal administration, the compositions can take the form of tablets or lozenges formulated in conventional manner.

For topical administration, the compound can be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal, oral or pulmonary administration.

For administration by inhalation, compounds can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of gelatin, for example, for use in an inhaler or insufflator may be formulated containing a powder mix of the compound and a suitable powder base such as lactose or starch.

The compounds, when it is desirable to deliver them systemically, can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

A compound can be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. Suitable means for parenteral administration include needle (including microneedle) injectors, needle-free injectors and infusion techniques.

Parenteral formulations can be aqueous solutions which can contain carriers or excipients such as salts, carbohydrates and buffering agents (such as at a pH of from 3 to 9), but, for some applications, they may be more suitably formulated as a sterile non-aqueous solution or as a dried form to be used in conjunction with a suitable vehicle such as sterile, pyrogen-free water. In other embodiments, any of the liquid formulations described herein can be adapted for parenteral administration of the compounds described herein. The preparation of parenteral formulations under sterile conditions, for example, by lyophilization under sterile conditions, can readily be accomplished using standard pharmaceutical techniques well-known to those skilled in the art. The solubility of a compound in a parenteral formulation can be increased by the use of appropriate formulation techniques, such as the incorporation of solubility-enhancing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethylcellulose, sorbitol, or dextran. Optionally, the suspension can also contain suitable stabilizers or agents which increase the solubility of the compounds to allow for the preparation of highly concentrated solutions.

Formulations for parenteral administration can be formulated for immediate and/or modified release. Active agents (i.e., the compounds) can be administered in a time-release formulation (e.g., in a composition which includes a slow-release polymer). The active agents can be prepared with carriers that will protect the compound against rapid release, such as a controlled-release formulation, including implants and microencapsulated delivery systems. Biodegradable, biocompatible polymers can be used (e.g., ethylene vinyl acetate, polyanhydrides, polyglycolic acid, collagen, polyorthoesters, polylactic acid and polylactic, polyglycolic copolymers (PGLA)). Methods for the preparation of such formulations are generally known to those skilled in the art. In other embodiments, the compounds in accordance with the present teachings or compositions comprising the compounds can be continuously administered, where appropriate.

Alternatively, the active compounds can be in powder form for constitution with a suitable vehicle, e.g., sterile pyrogen-free water, before use.

The compounds can also be formulated in rectal or vaginal compositions such as suppositories or retention enemas, e.g., containing conventional suppository bases such as cocoa butter or other glycerides.

In addition to the formulations described above, a compound can also be formulated as a depot preparation. Such long-acting formulations can be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers, such as PEGs.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, R., Science 249:1527-33 (1990).

The compound and optionally other therapeutics can be administered per se (neat) or in the form of a pharmaceutically acceptable salt. When used in medicine the salts should be pharmaceutically acceptable, but non-pharmaceutically acceptable salts can conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions contain an effective amount of a compound and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also can be commingled with the compounds, and with each other, in a manner such that there is no interaction which would substantially impair the desired pharmaceutical efficiency.

The therapeutic agent(s), including specifically, but not limited to, a compound, can be provided in particles. Particles as used herein means nanoparticles or microparticles (or larger particles) which can consist in whole or in part of the compound or the other therapeutic agent(s) as described herein. The particles can contain the therapeutic agent(s) in a core surrounded by a coating, including, but not limited to, an enteric coating. The therapeutic agent(s) also can be dispersed throughout the particles. The therapeutic agent(s) also can be adsorbed into the particles. The particles can be of any order release kinetics, including zero-order release, first-order release, second-order release, delayed release, sustained release, immediate release, and any combination thereof, etc. The particle can include, in addition to the therapeutic agent(s), any of those materials routinely used in the art of pharmacy and medicine, including, but not limited to, erodible, nonerodible, biodegradable, or nonbiodegradable material or combinations thereof. The particles can be microcapsules which contain the compound in a solution or in a semi-solid state. The particles can be of virtually any shape.

Both non-biodegradable and biodegradable polymeric materials can be used in the manufacture of particles for delivering the therapeutic agent(s). Such polymers can be natural or synthetic polymers. The polymer is selected based on the period of time over which release is desired. Bioadhesive polymers of particular interest include bioerodible hydrogels described in Sawhney et al., Macromolecules 26:581-587 (1993), the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) can be contained in controlled release systems. The term “controlled release” is intended to refer to any drug-containing formulation in which the manner and profile of drug release from the formulation are controlled. This refers to immediate as well as non-immediate release formulations, with non-immediate release formulations including, but not limited to, sustained-release and delayed-release formulations. The term “sustained release” (also referred to as “extended release”) is used in its conventional sense to refer to a drug formulation that provides for gradual release of a drug over an extended period of time, and that can result in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant can be particularly suitable for treatment of chronic conditions. “Long-term” release, as used herein, means that the implant is constructed and arranged to deliver therapeutic levels of the active ingredient for at least 7 days, and up to 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the disclosure contained herein in view of information known to the ordinarily skilled artisan, and may be made without departing from the scope of the disclosure or any embodiment thereof.

### EXAMPLES

### Example 1: Synthesis of a Dual-FAP Pre-Functionalized Conjugate of the Formula (Q-L^{Q})₂-Y-L^{X}, where L^{X} is a Resin-Bound Spacer

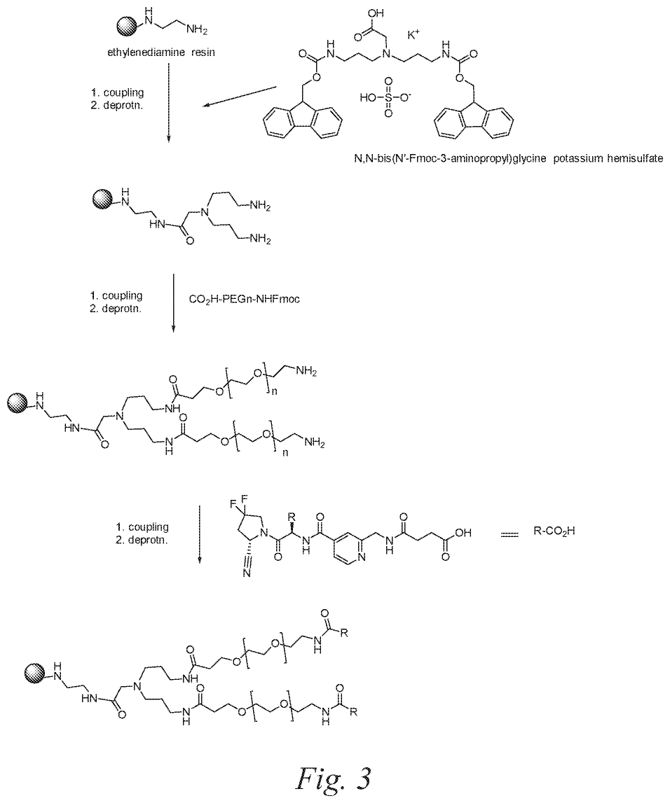

A representative synthetic scheme is outlined in FIG. 3, showing the synthesis of a resin-bound multivalent conjugate intermediate. To synthesize the conjugate intermediate of FIG. 3, a solution of anhydrous DMF, N,N-bis(N′-Fmoc-3-aminopropyl)glycine potassium hemisulfate (1 eq), 1-[Bis(dimethylamino)methylene]-1H-1,2,3-triazolo[4,5-b]pyridinium 3-oxide hexafluorophosphate (HATU) (2.5 eq), and anhydrous N,N-diisopropylethylamine (DIPEA) (5 eq) was combined with an ethylenediamine resin and stirred under argon atmosphere for 6 h. The coupling mix was washed from the resin, giving a resin-bound, Fmoc-protected glycine derivative. The resin was resuspended in a solution of anhydrous DMF and piperidine or piperazine, which was washed with excess DMF. The resin was then mixed with a solution of anhydrous DMF, CO₂H-PEGn-NHFmoc (2 eq), HATU (2.5 eq), and anhydrous DIPEA (5 eq) and stirred at r.t. for 6 h. The coupling solution was washed with solvent and the Fmoc groups were removed with a solution of piperidine as described previously. The deprotected amines were coupled to the carboxy tail of the FAP ligand shown in FIG. 3 by combining resin, FAP ligand (2 eq), HATU (2.5 eq), anhydrous DIPEA (5 eq), and anhydrous DMF while stirring at r.t. for 6 h. Following the final coupling step, the resin was washed with excess solvent (DMF) to remove residual coupling reagents.

### Example 2: Synthesis of a Dual-FAP Rhodamine Dye Conjugate 5b

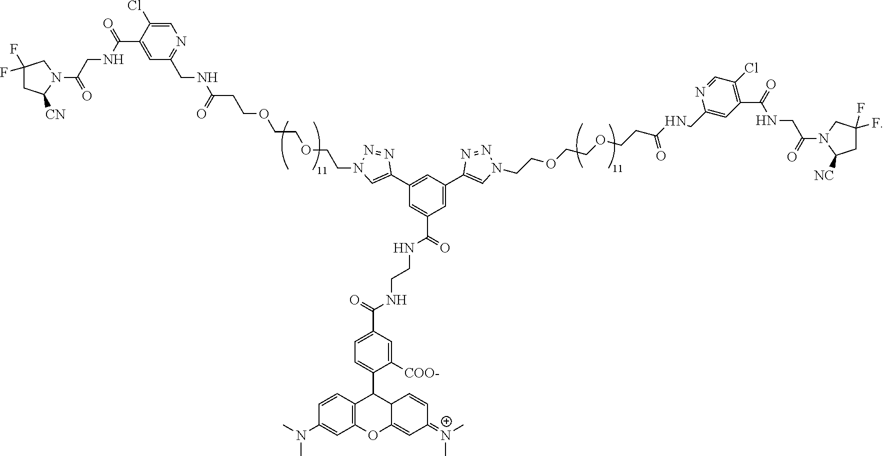

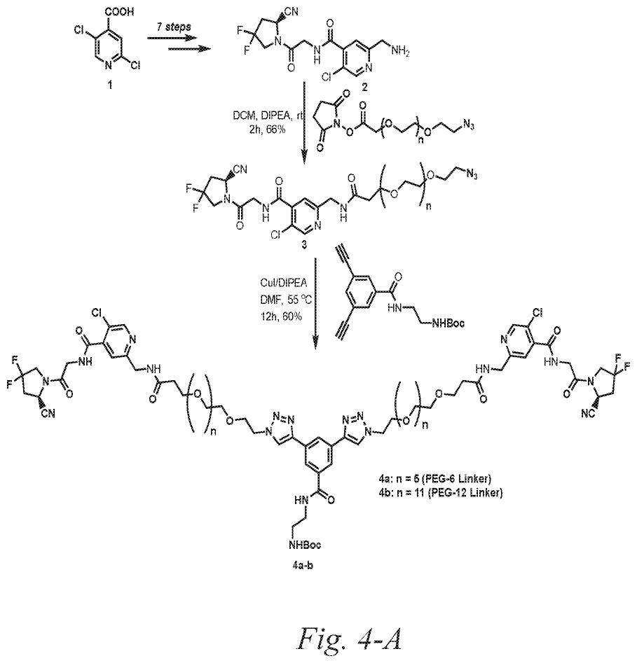

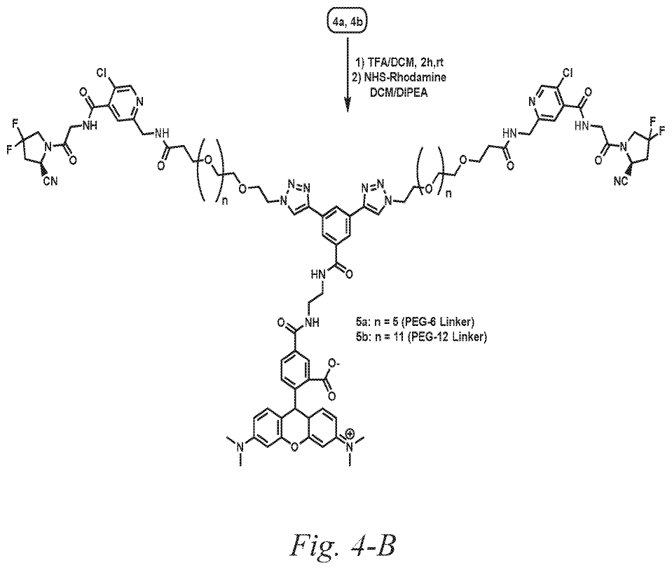

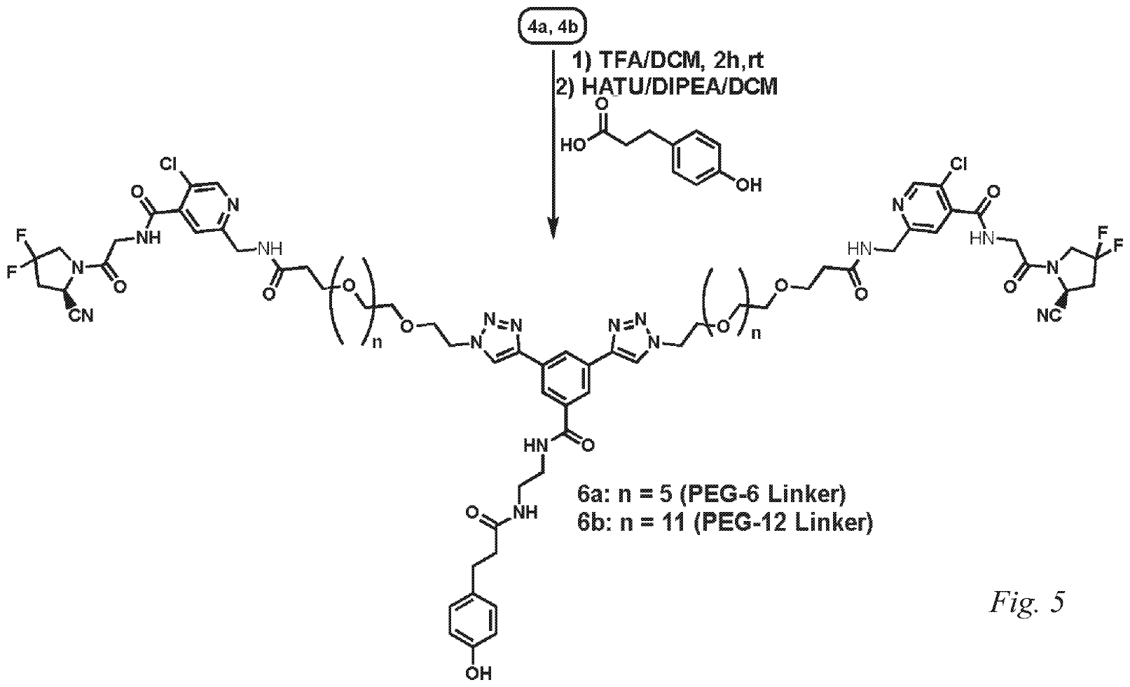

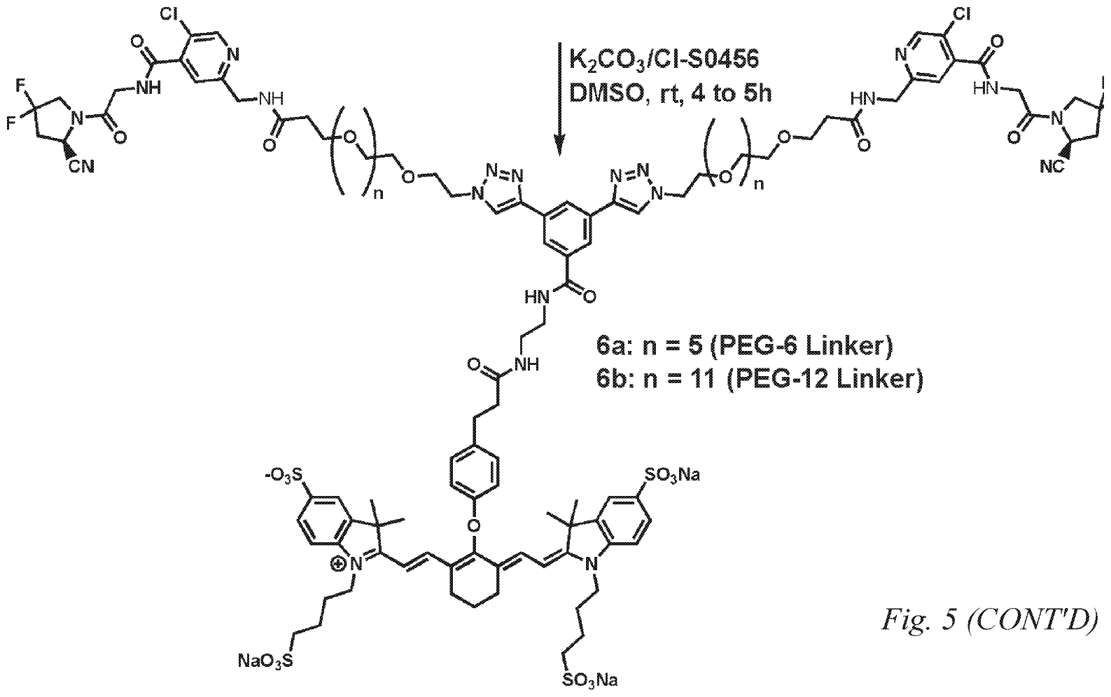

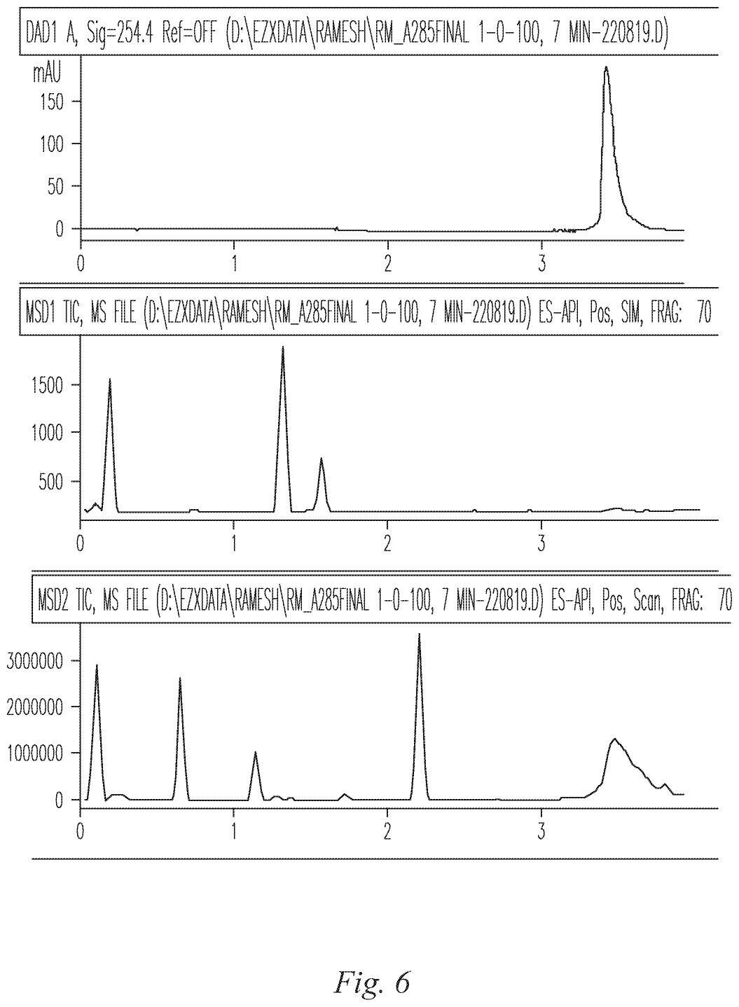

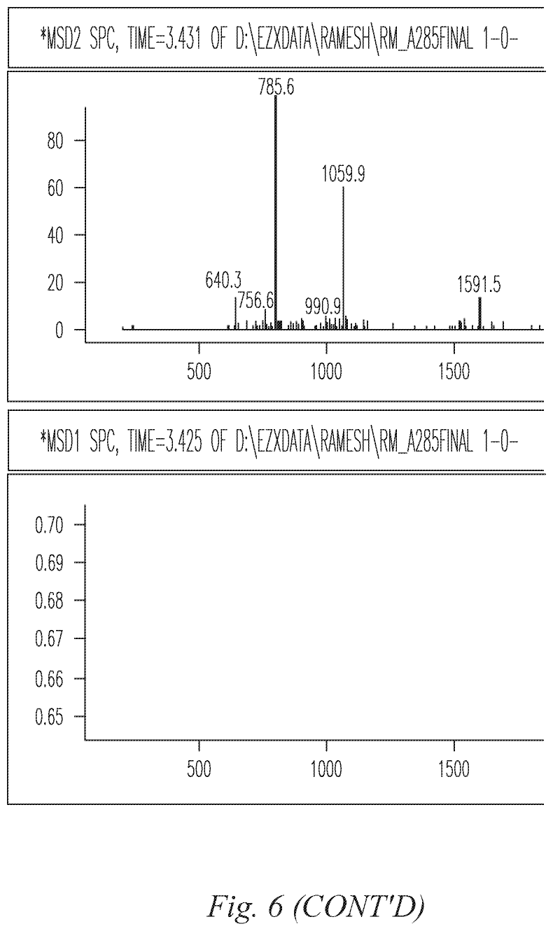

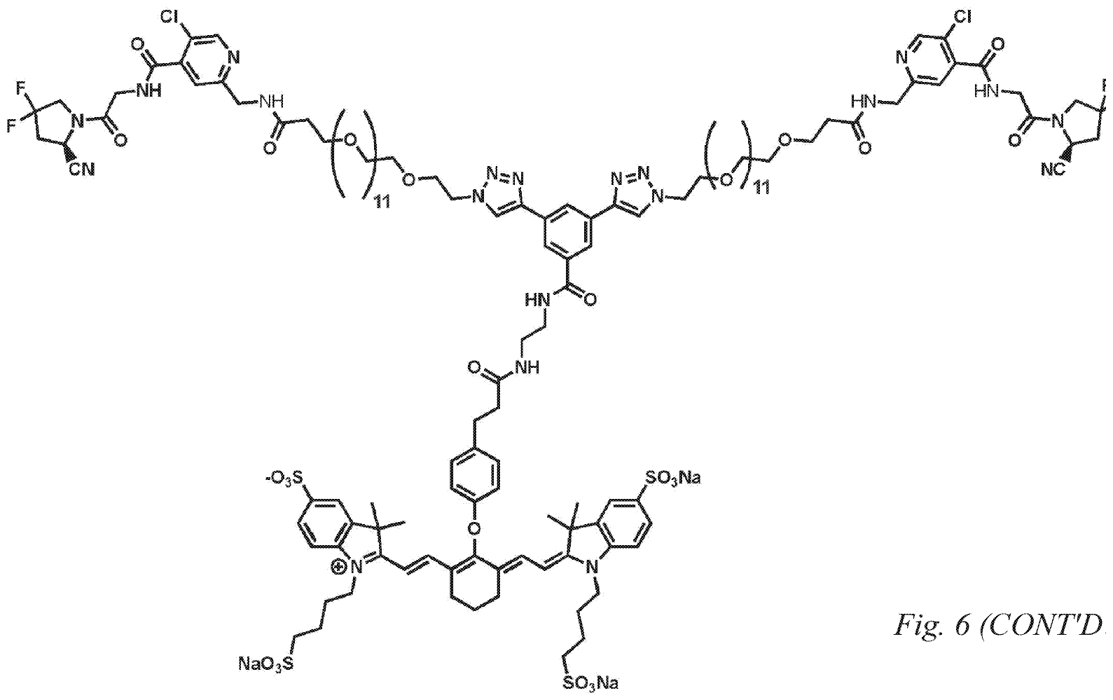

A multivalent conjugate (or compound) comprising two FAP-targeting ligands Q was synthesized as described in FIGS. 4A and 4B. Starting with a substituted pyridine, a FAP-targeting ligand such as Q^{A} was synthesized in 7 steps. The FAP ligand was coupled to an NHS PEG azide moiety using conditions similar to those described in Example 1. FAP ligand, NHS PEG azide, anhydrous DIPEA, and DCM were combined and stirred at r.t. for 2 h. The desired product was isolated in a 66% yield. The FAP-azide was then coupled to a di-alkyne core by combining the FAP azide intermediate, tert-butyl (2-(3,5-diethynylbenzamido)ethyl)carbamate, CuI, and anhydrous DIPEA in anhydrous DMF. The solution was heated to 55° C. for 12 h, resulting in a 60% yield of the desired Boc-protected intermediate. The Boc-protected intermediate (e.g. 4a or 4b) was treated with a mixture of trifluoroacetic acid (TFA) and DCM and stirred at r.t. for 2 h, resulting in cleavage of the Boc group. Lastly, to the deprotected intermediate was added NHS-Rhodamine, DCM, and DIPEA, resulting in compounds 5a or 5b.

### Example 3. Synthesis of a Dual-FAP Near-Infrared S0456 Dye Conjugate 6b

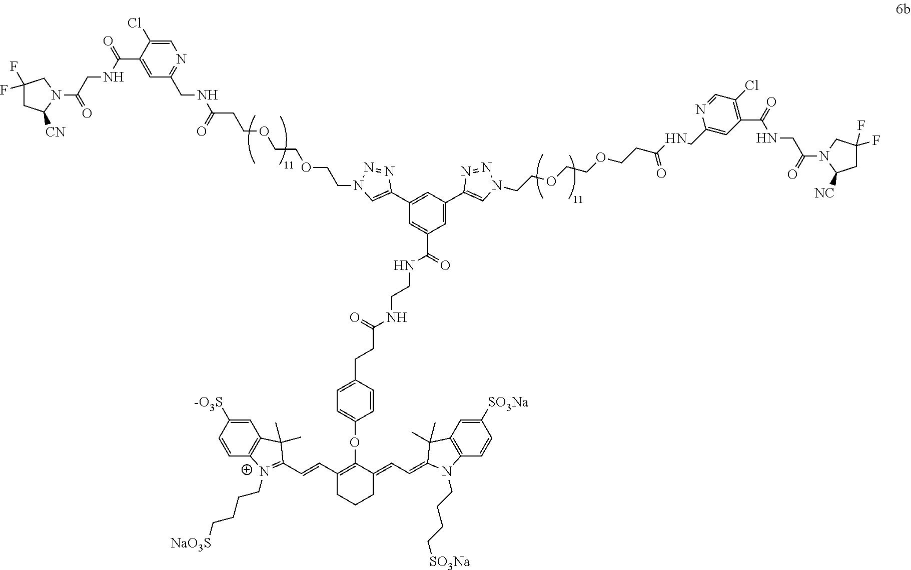

Intermediates 4a and 4b from Example 2 and FIG. 4A were used as the starting materials in the synthesis of compounds 6a and 6b. Intermediate 4b was first Boc-deprotected by combining 4b with a solution of TFA and DCM and stirring at r.t. for 2 h. The desired Boc-deprotected 4b intermediate was then combined with 3-(4-hydroxyphenyl)propanoic acid, HATU, DIPEA, and DCM using conditions similar to those described previously. The product of the coupling reaction was subsequently treated with K₂CO₃, and CI-S0456 in a DMSO solution and stirred at r.t. for 4-5 h, yielding compound 6b. Compound 6a was synthesized using similar conditions.

### Example 4. Synthesis of Dual-FAP Multivalent Conjugates

Using a similar process as described in Examples 1-3, various compounds, such as those below and in Table 9, are synthesized.

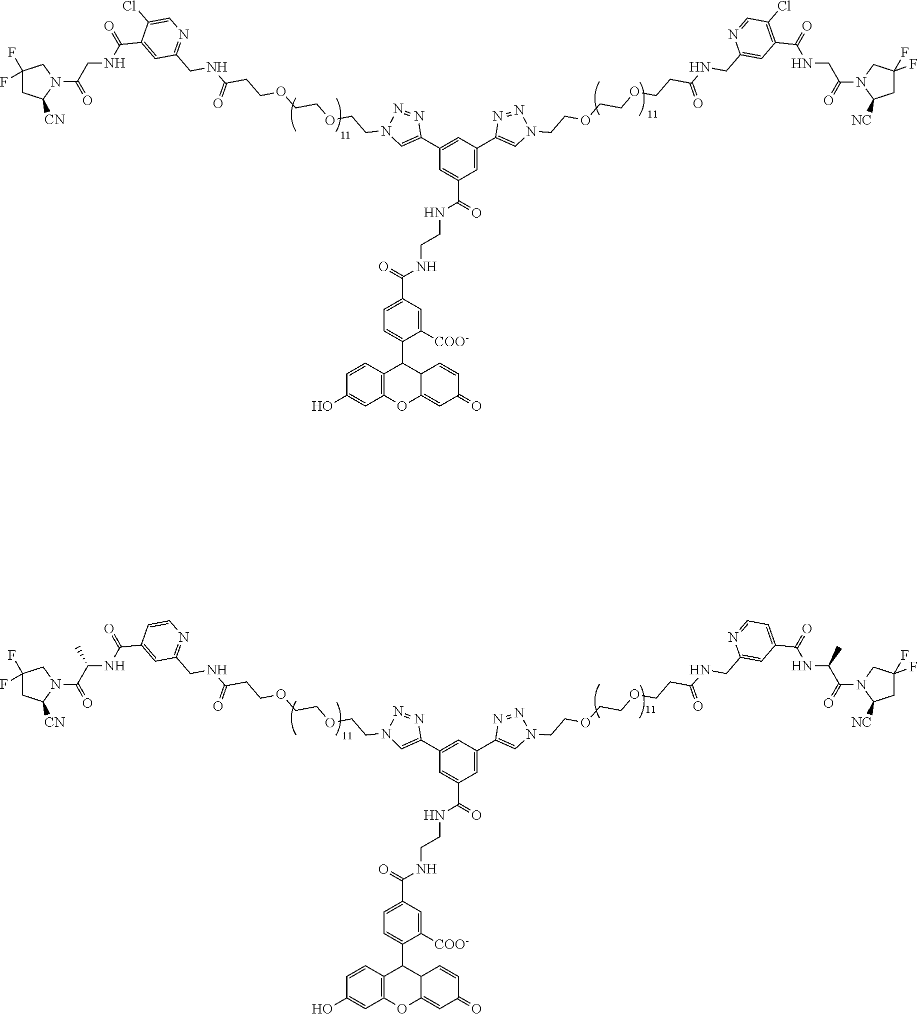

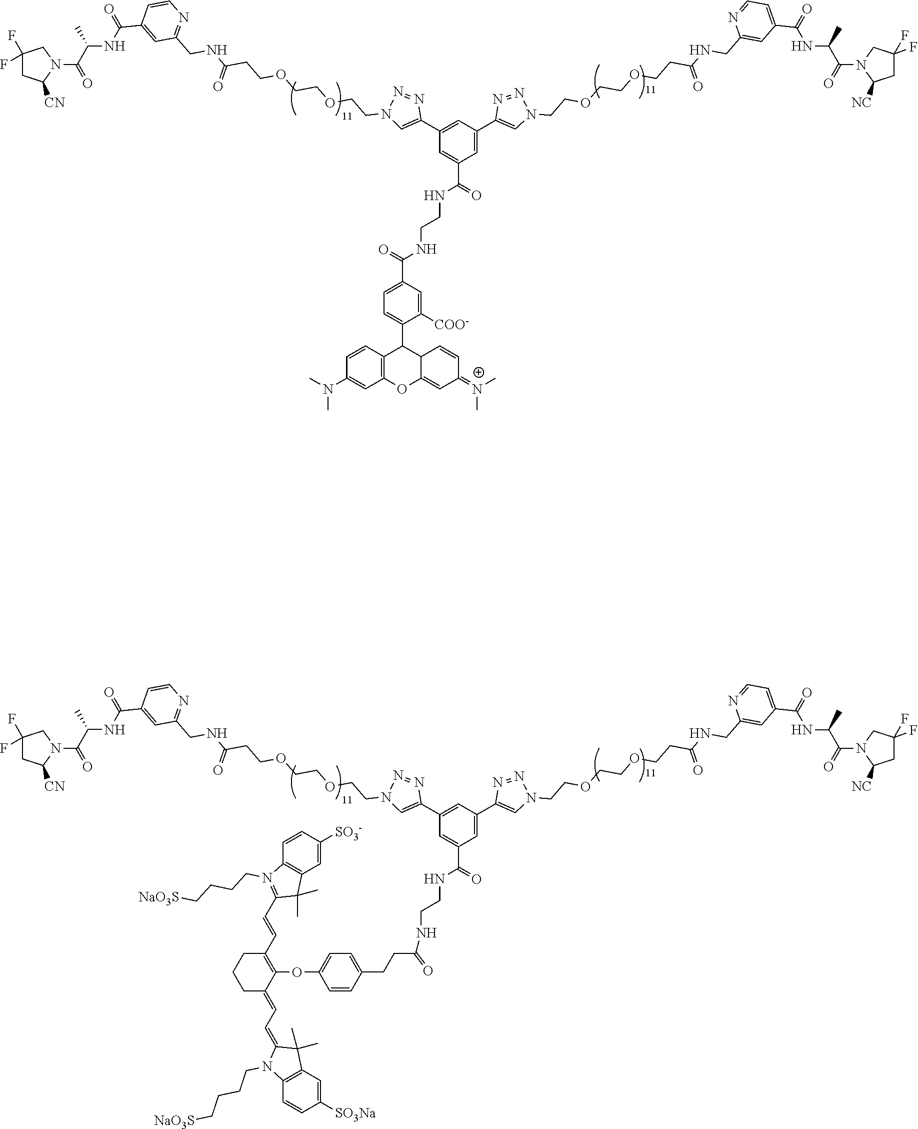

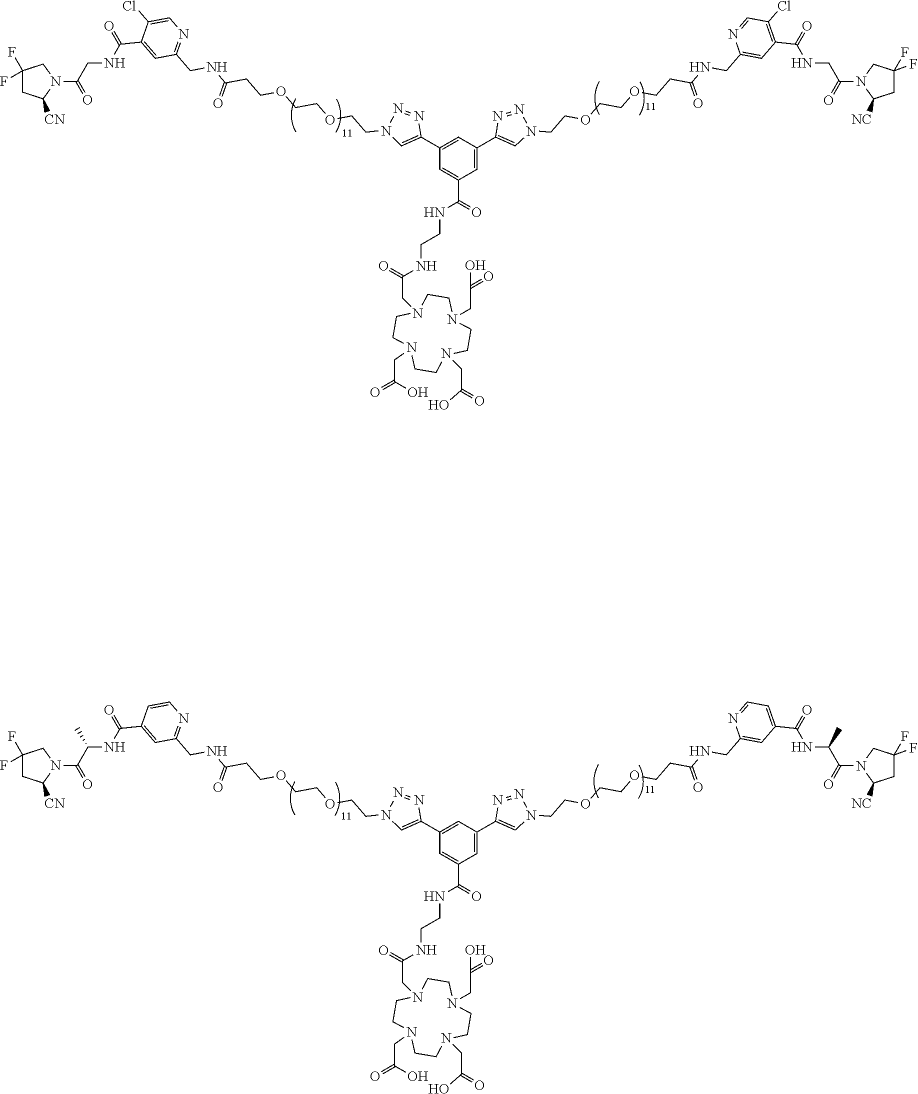

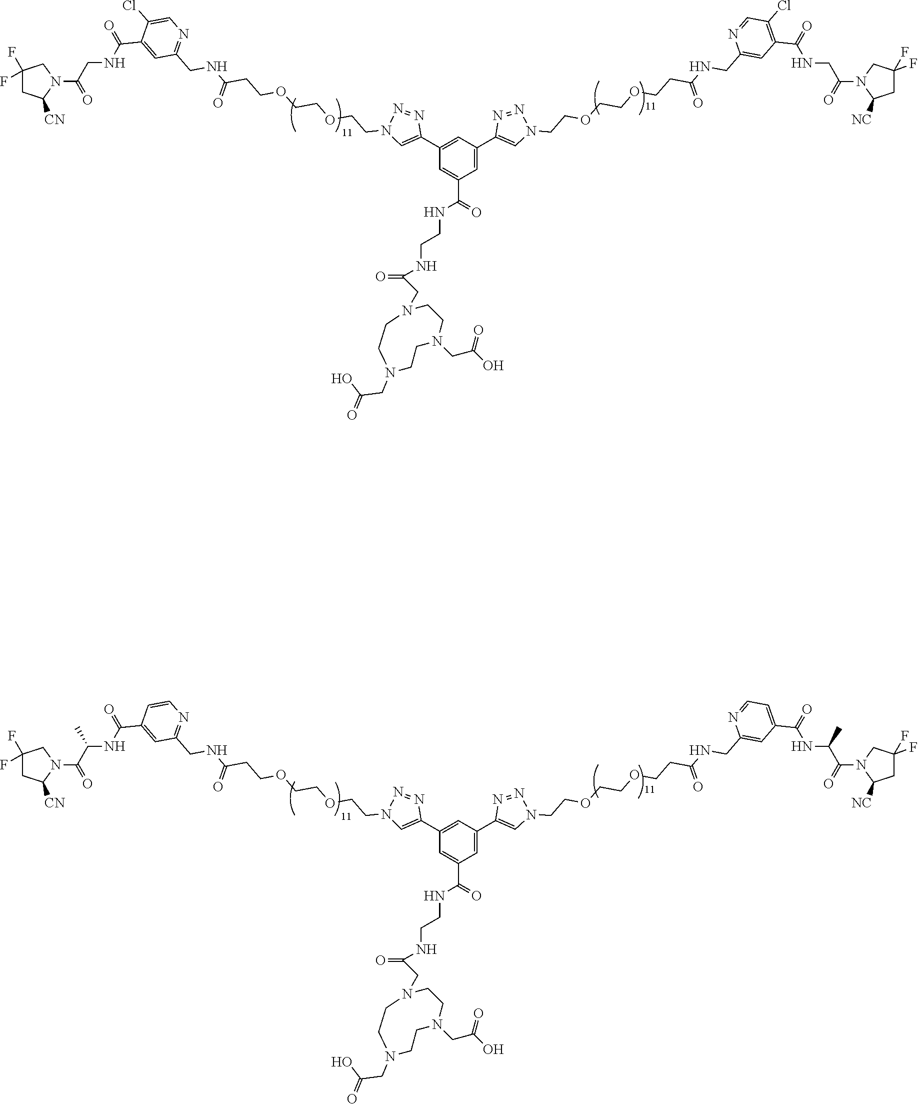

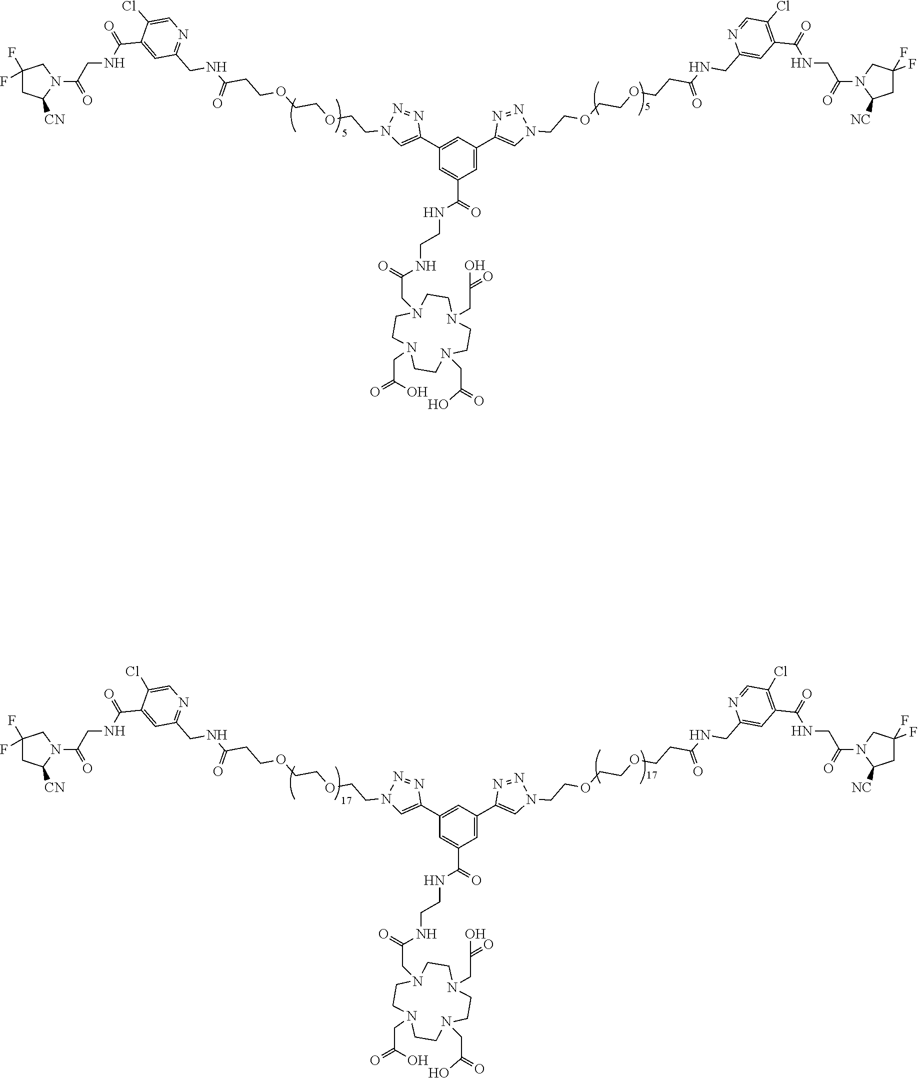

### Example 5. Binding Studies for Mono-FAP and Dual-FAP Targeting Ligands

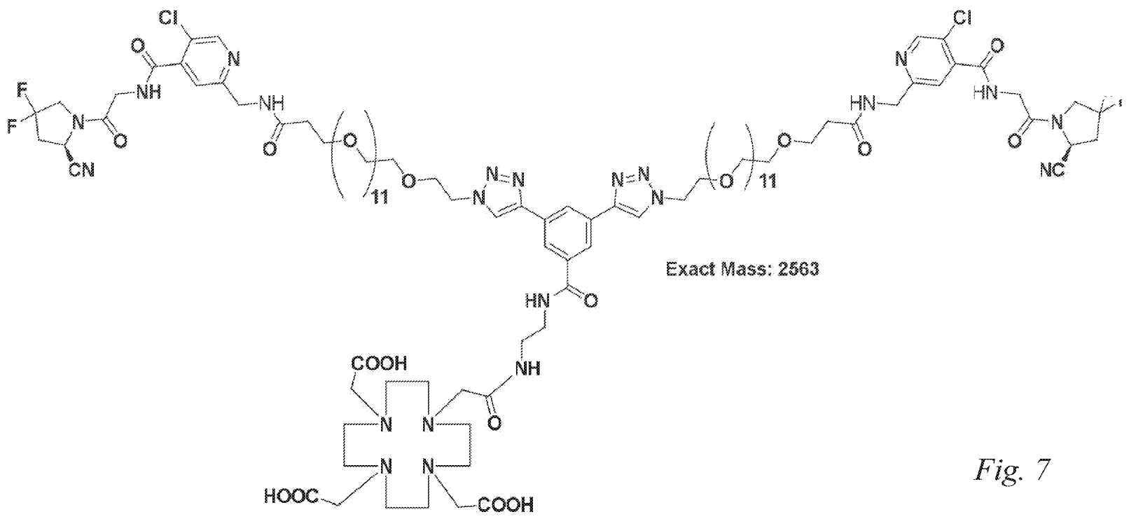

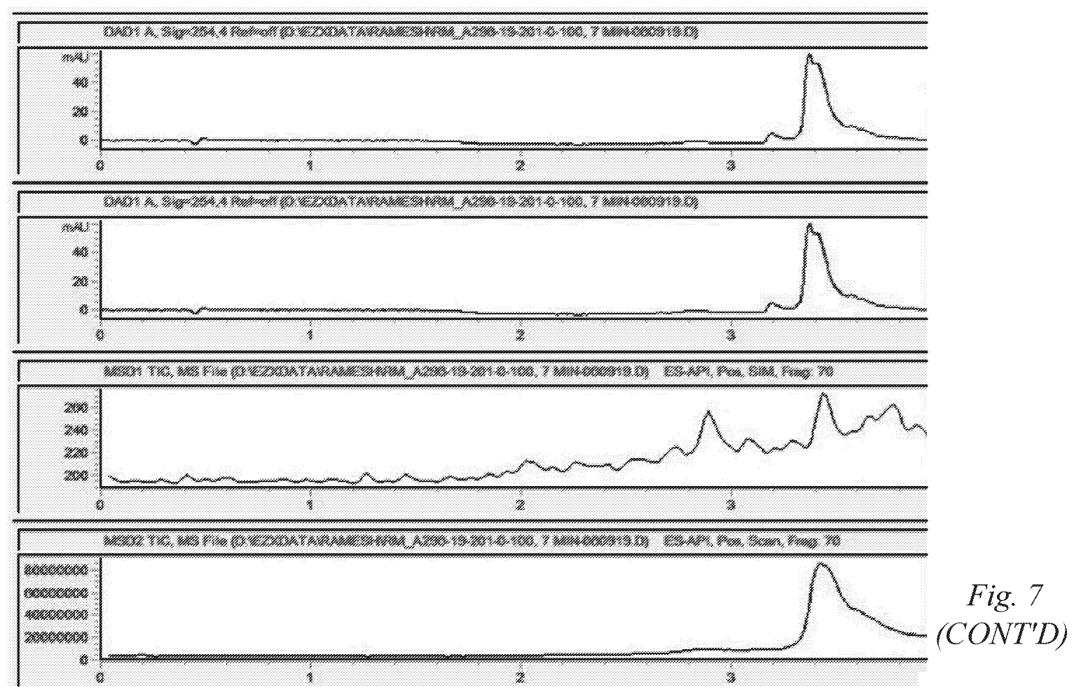

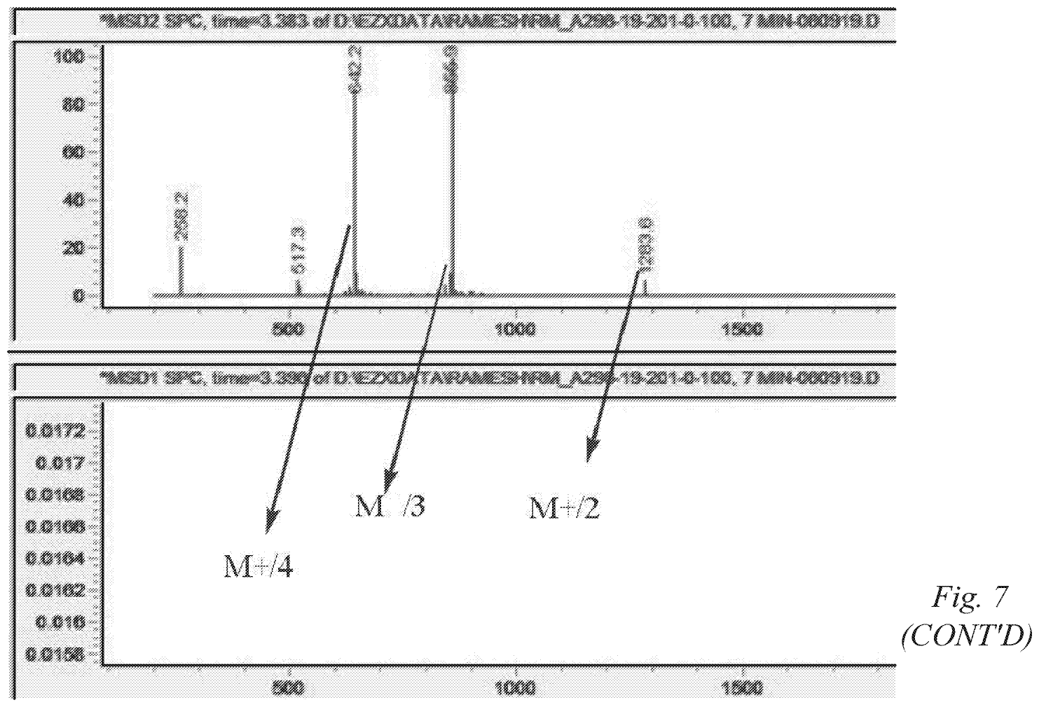

In this example, HT1080-FAP cells were seeded in 4 well confocal plates at 37° C. for 12 h. Cell growth media were removed, and the cells were incubated with FAP-ligand conjugates at concentration ranging from 3.0 nM (lowest) to 25 nM (highest) in 1% FBS in PBS. After incubation at 37° C. for 1 h, PBS was replaced with cell growth media and cells were incubated at 37° C. for 8 h to 48 h. The cells were washed with cold 1% FBS in PBS (3×300 uL). Images were acquired using confocal microscopy at 1 h and 8 h time points (FIG. 7), and 24 h and 48 h time points (FIG. 8). The dual-FAP-targeting conjugate was retained in cells up to 48 h following treatment with the conjugate, while mono-FAP-targeting conjugate was cleared by 24 h.

### Example 6. Binding Studies of Dual-FAP-Targeting Conjugates on Non-FAP HT1080 Cells

HT1080 cells not expressing FAP were used to study the binding of dual-FAP-targeting conjugates. As indicated in FIG. 9, binding of a dual-FAP-targeting conjugate was FAP-specific and the conjugates did not bind to non-FAP expressing HT1080 cells despite higher concentration of conjugates used (25 nM). FIG. 9 shows no detectable retention of the dual-FAP ligand at either 12.5 nM or 25 nM.

### Example 7. In Vivo Imaging of Dual-FAP Conjugate 6b on KB Tumor Bearing Mice

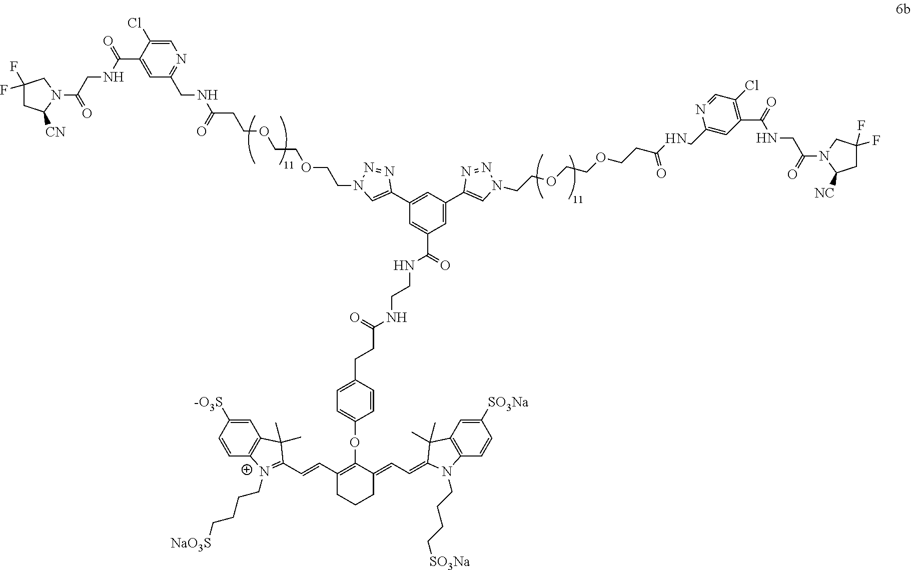

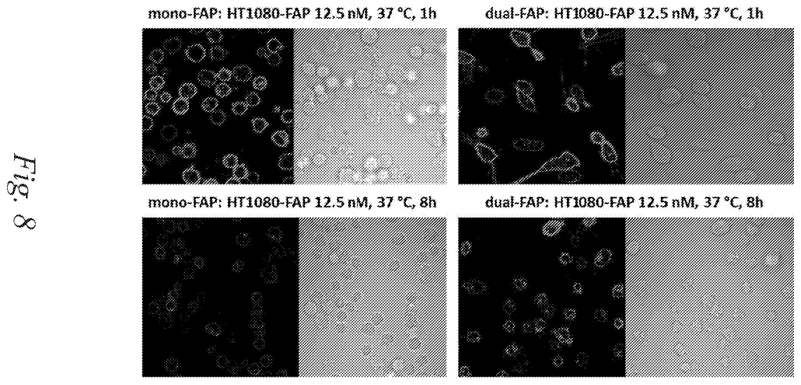

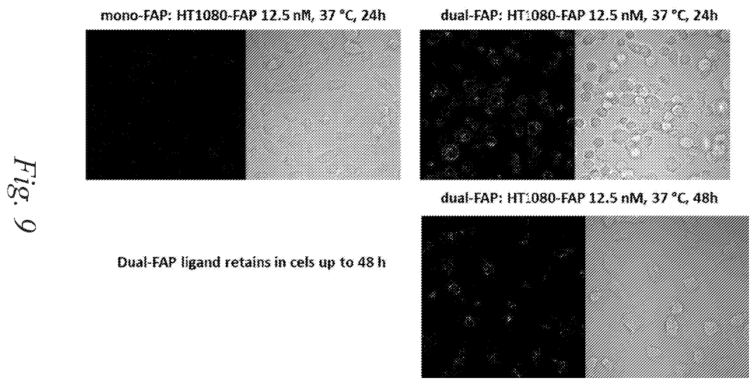

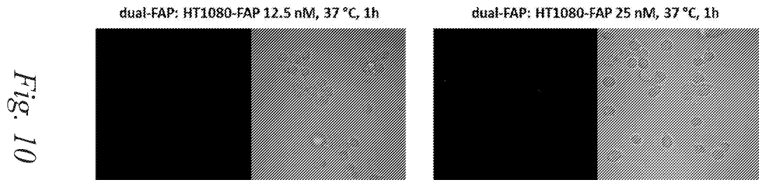

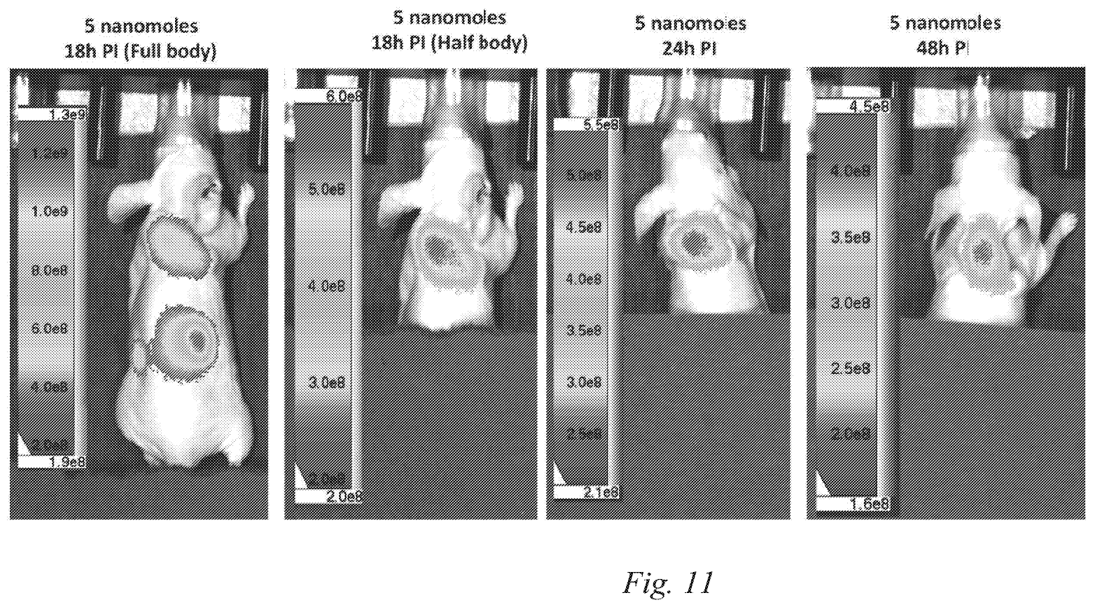

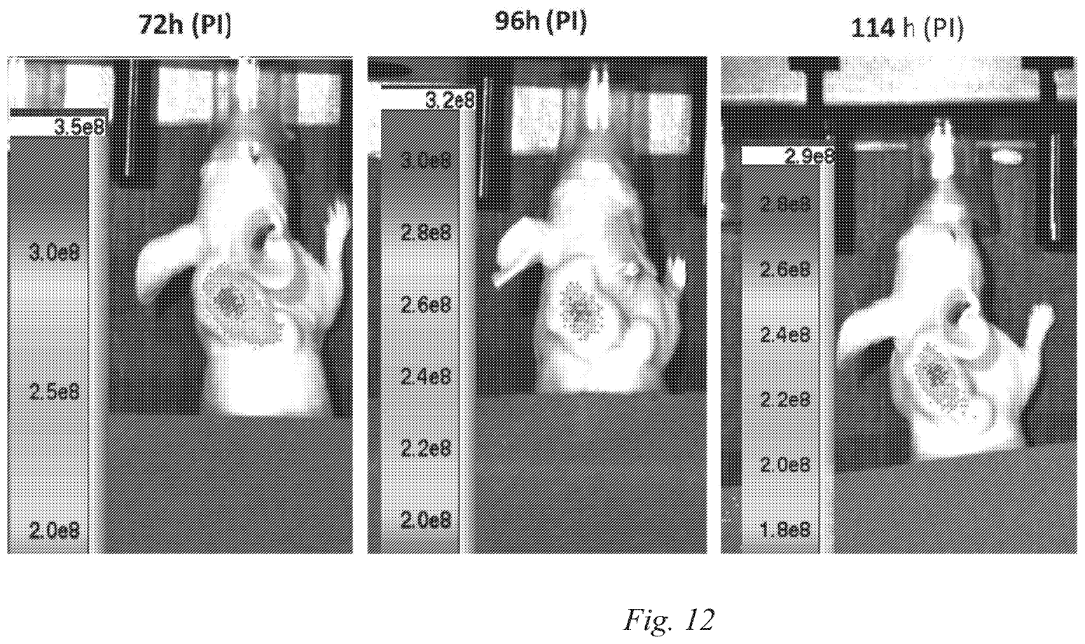

KB cells were cultured in RPMI (with 10% FBS, 1% penicillin streptomycin) growth media. 4 million KB cells/mouse were implanted in athymic female nude mice at the right shoulder (subcutaneous injection) and maintained until the tumor size reached 250-300 mm³. A dual-FAP-targeting conjugate 6b with the structure captioned above was prepared at 5 nanomoles per injection, diluted in 100 μL solution in PBS. Conjugate 6b was injected into KB cell-bearing mice via tail vein injection. Images of mice were acquired at different time points at 745/810 nm (excitation/emission). FIG. 10 shows a time course imaging study with dual-FAP conjugate 6b on KB bearing mice. FIG. 11 shows that dual-FAP-targeted S0456 was retained in KB tumors for up to 4 days. The biodistribution of dual-FAP-targeted SO456 at 114 hours post-injection is shown in FIG. 12, at left is the full body image, and at right, the kidney is covered by the imaged ligand.

### Example 8. In Vivo Evaluation of Dual-FAP Compound 7b on KB Tumor Bearing Mice

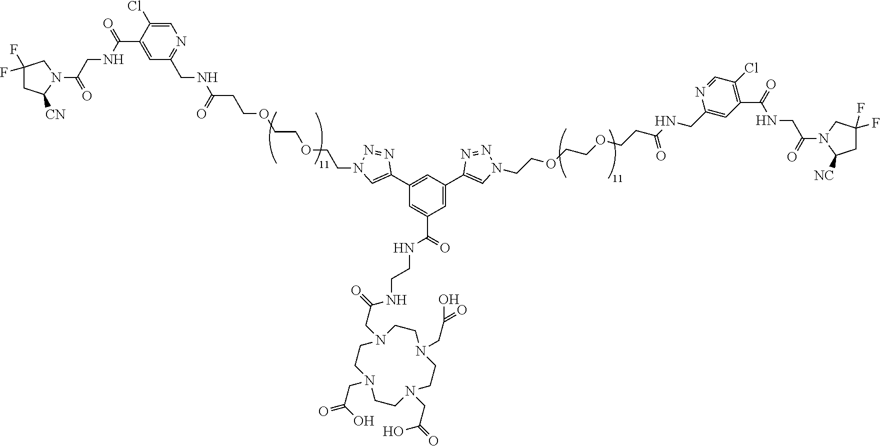

Using methods similar to those described in Example 7, compound 7b is evaluated in KB tumor bearing mice. In this example, 7b is ^{99m}Tc-DOTA-7b, wherein the DOTA moiety chelates ^{99m}Tc. On day 1, mice are treated with conjugate 6b as described in Example 7, to image tumor size. On day 5, and every fourth day thereafter for a period of 3 cycles, mice are treated with ^{9mm}Tc-DOTA-7b. On day 17, compound 6b is again administered as previously described to assess change in tumor size. Pre- and post-treatment images are taken to evaluate the efficacy of ^{9mm}Tc-DOTA-7b in reducing tumor size.

### Example 9

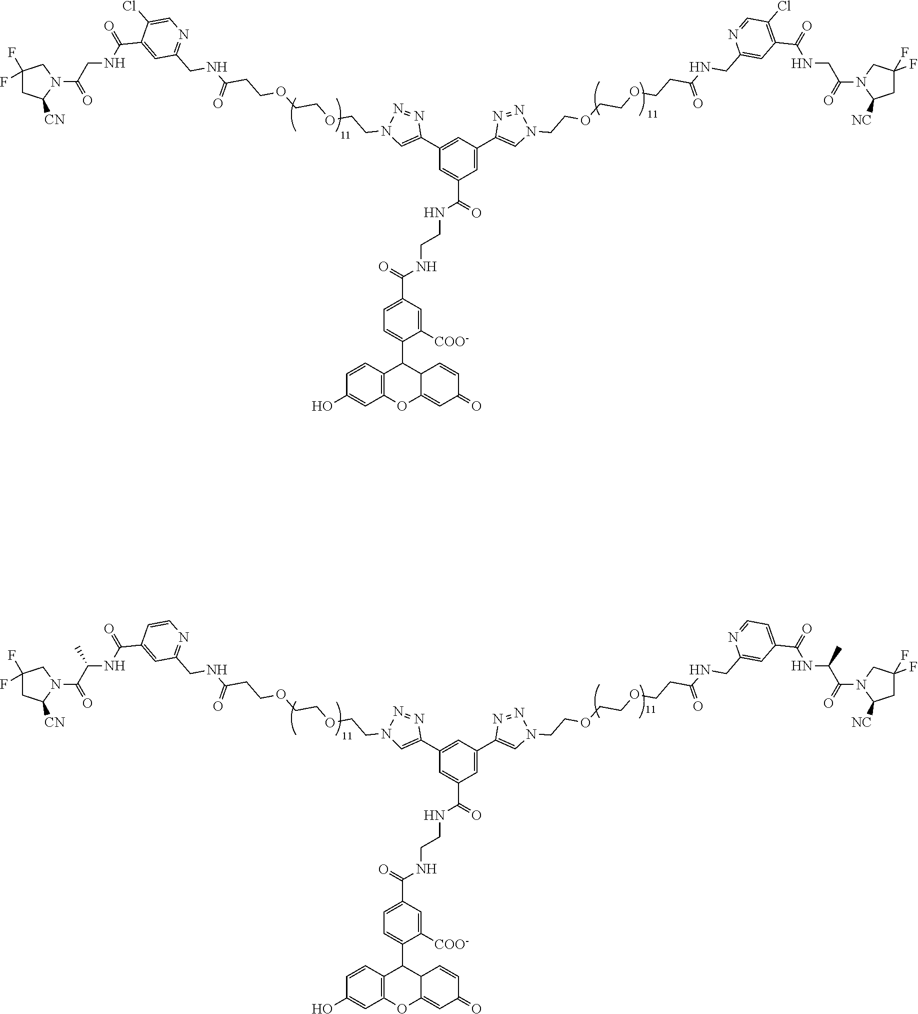

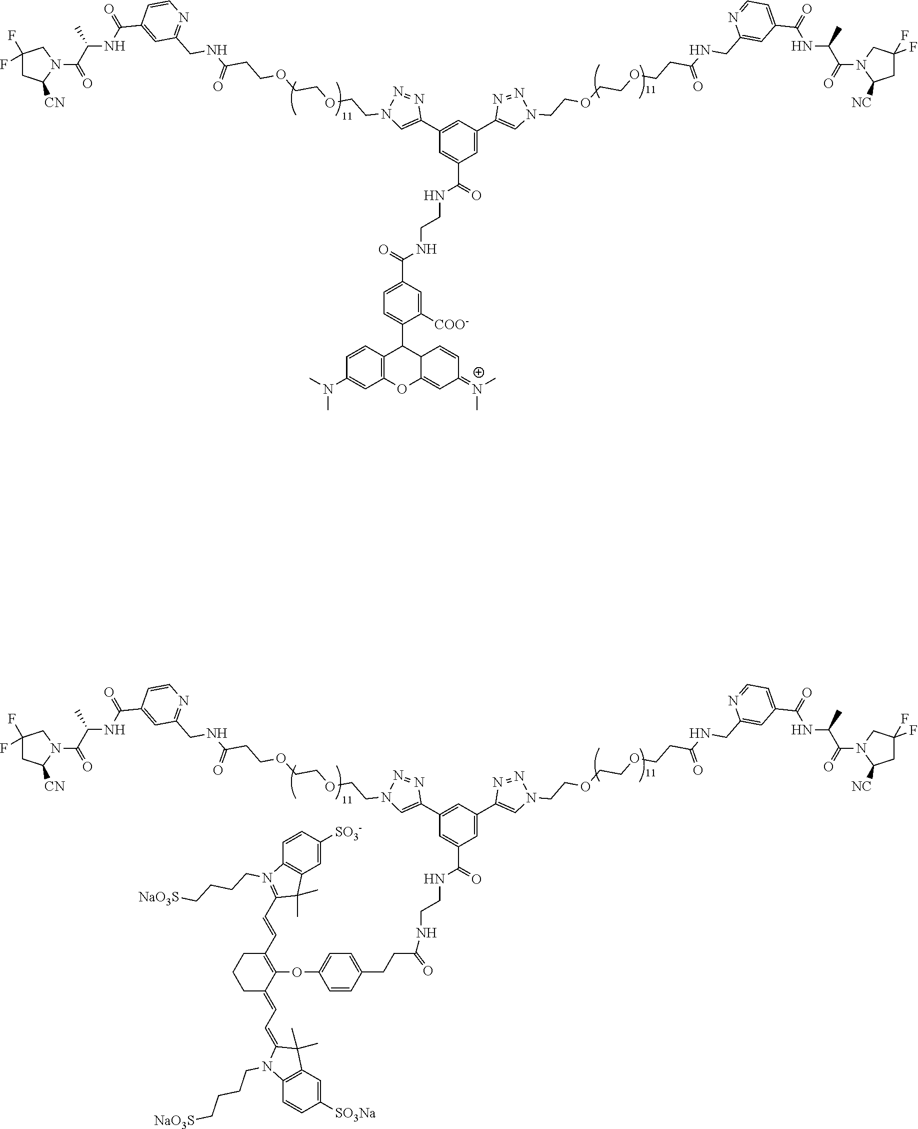

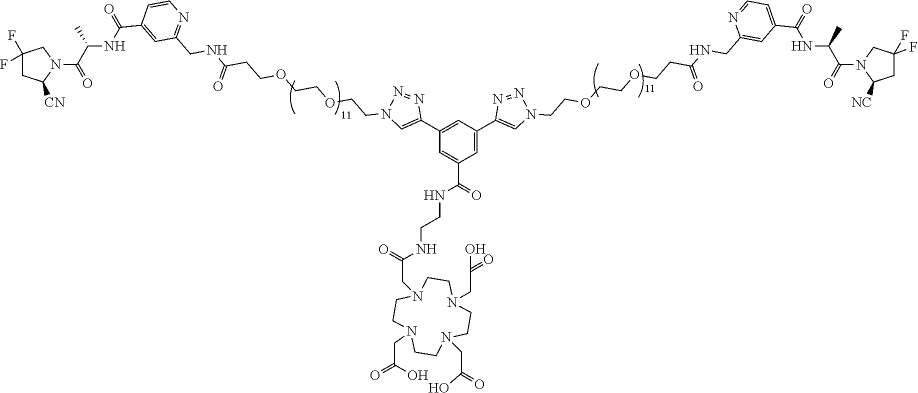

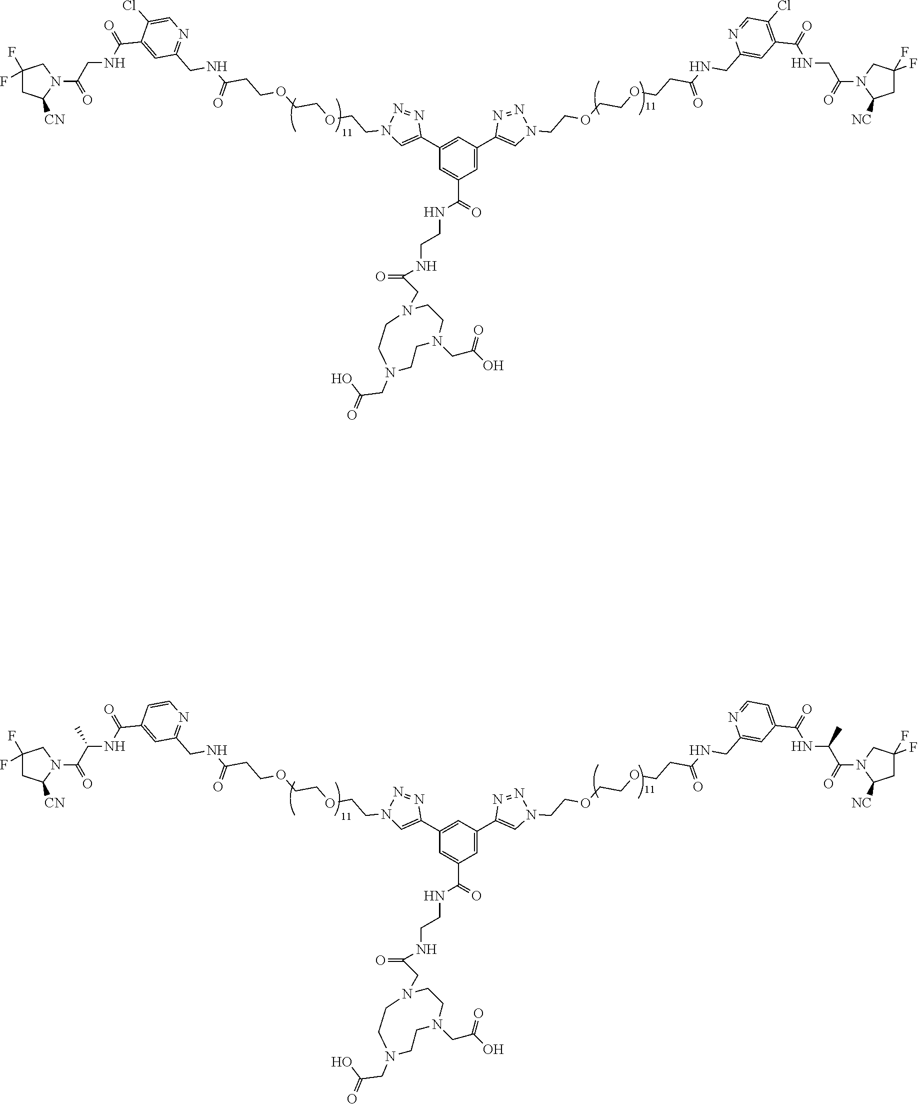

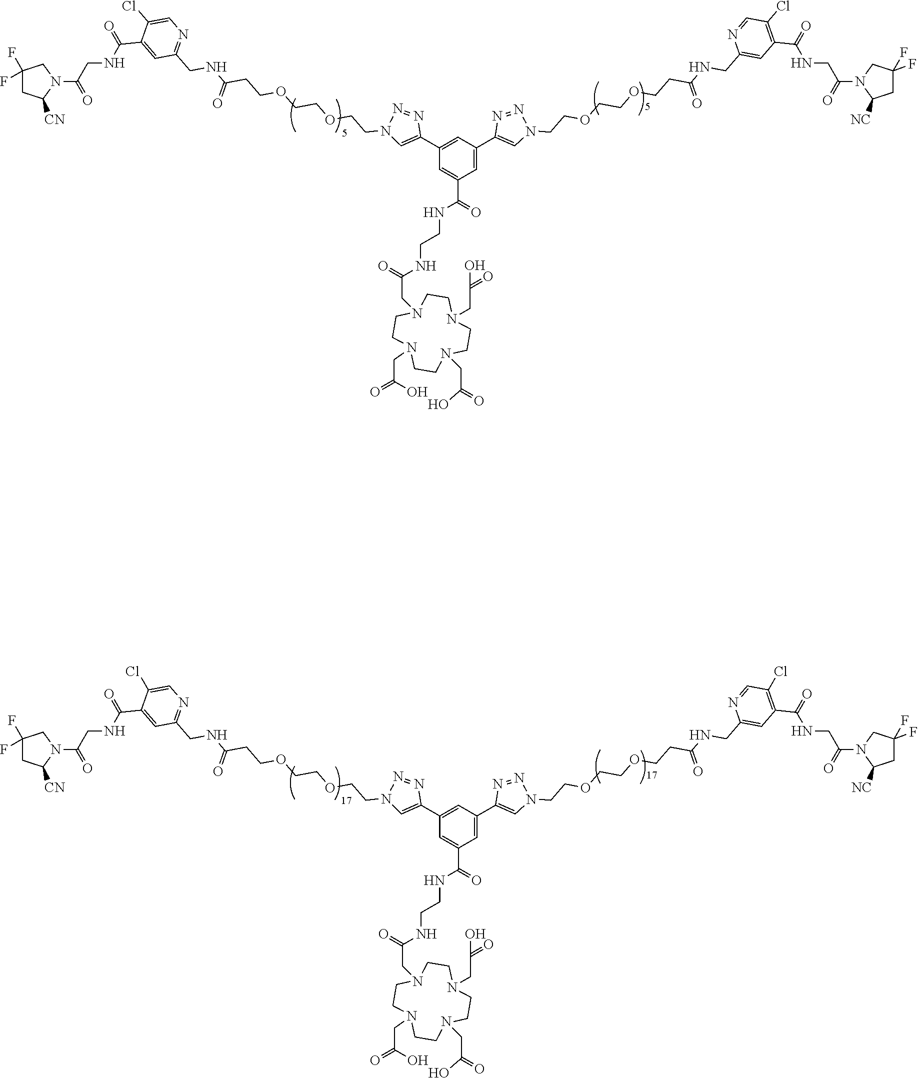

Compounds disclosed herein (e.g., those captioned above) are evaluated using methods consistent with those described in Examples 7 and 8 to determine the imaging and therapeutic efficacy of each compound. Spacer length, active agents, and FAP ligands are optimized based on the detectability and therapeutic efficacy of each trial.

### Example 10. Competition Experiment with Excess Free FAP Ligand without Imaging Dye Compared to Dual-FAP-Targeting Conjugate with Imaging Dye

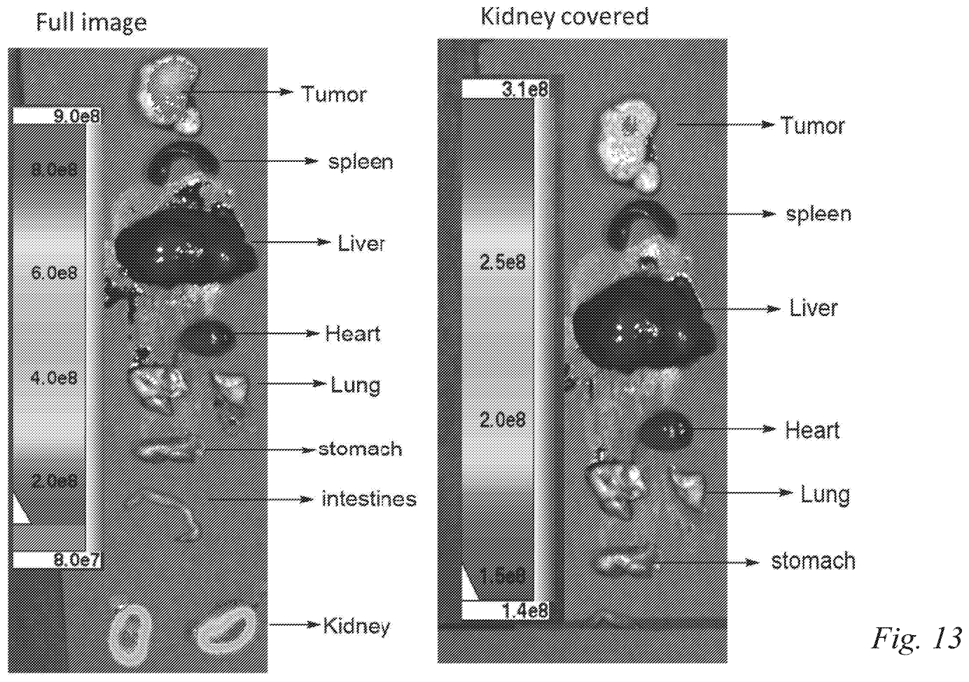

In this example, as shown in FIG. 13, each panel's left mouse was injected with a dual-FAP-targeting ligand conjugated with the imaging dye S0456, and each panel's right mouse was treated with free FAP ligand as a competition to the dual-FAP targeting conjugate. The mouse on the right in each panel was sequentially injected with 100-fold excess of 500 nanomoles competition ligand (i.e., free FAP ligand without S0456 dye), followed by 5 nanomole dual-FAP conjugate with S0456 dye. Images are acquired as described above 6 h post injection. Low fluorescence intensity at tumor site was observed in the competition mouse as compared to the targeted mouse (high intensity), which indicated the dual-FAP-targeting ligand was FAP-specific.

### Example 11. Monovalent Ligand Images at Different Time Points

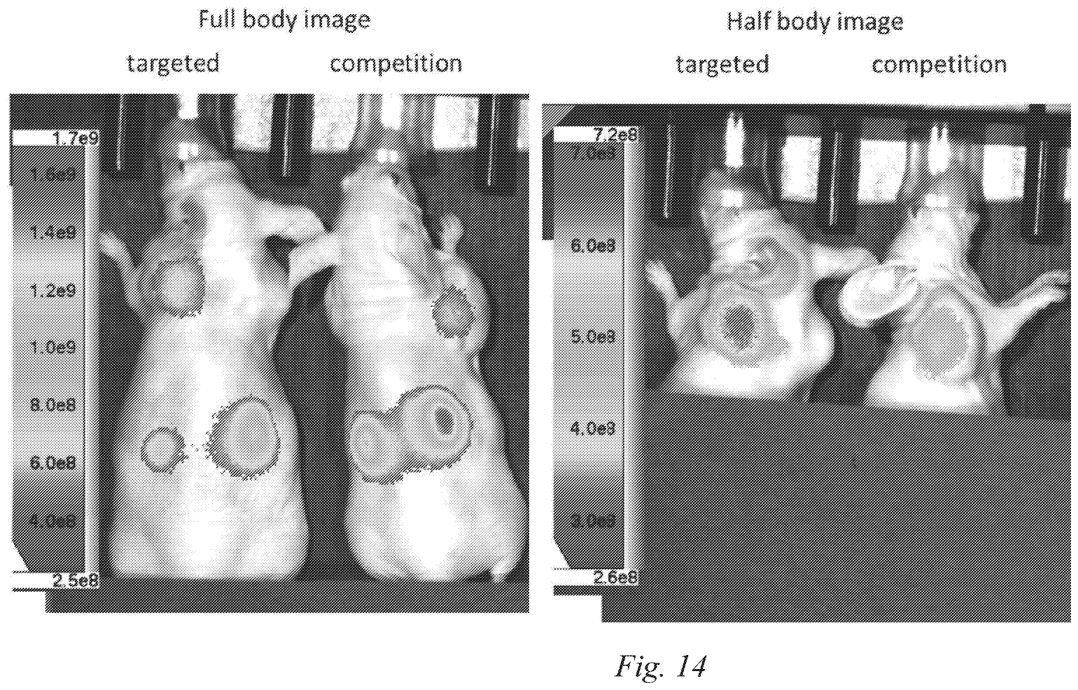

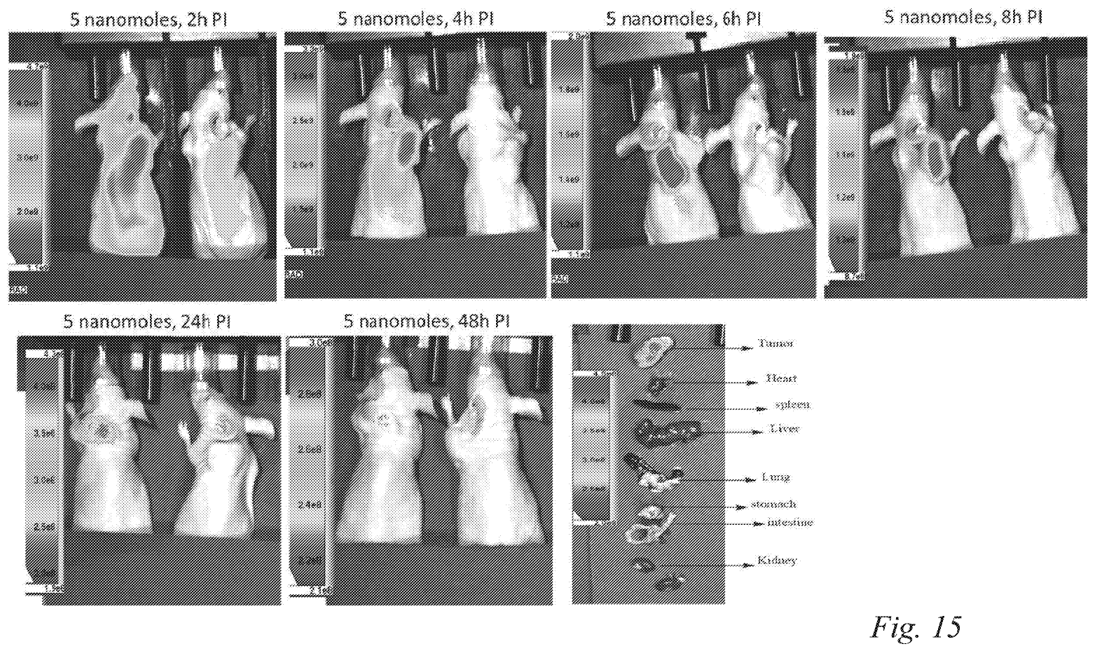

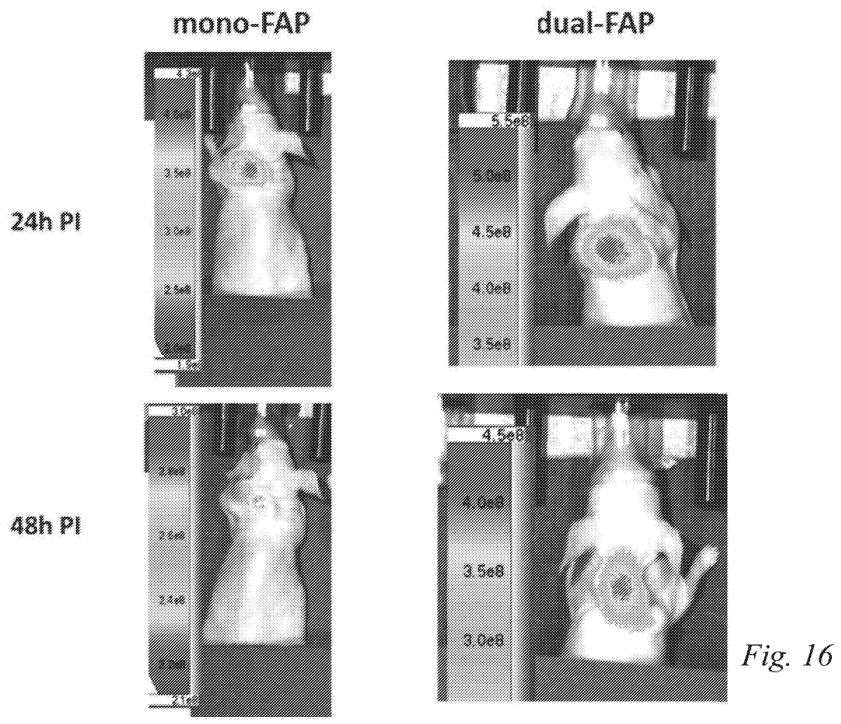

Athymic female nude mice were implanted with KB tumor cells until tumor size reached about 250-300 mm³. At extraction/emission of 745 nm/810 nm, imaging data were obtained. In FIG. 14, in each panel, the mouse on the left was targeted by injecting a mono-FAP-targeting ligand conjugated with the imaging dye S0456, and the mouse on the right was treated with free FAP ligand. The mouse on the right of each panel was sequentially injected with 100-fold excess of 500 nanomoles competition ligand (i.e., FAP ligand without S0456 dye), followed by 5 nanomole mono-FAP conjugate with S0456 dye. Images are acquired as described above at different time points post-injection. Complete absence of fluorescence at the tumor site in the competition mouse was observed as compared to the targeted mouse (high intensity), which indicated that the mono-FAP conjugate was highly FAP-specific. In FIG. 15, a comparison between mono-FAP and dual-FAP targeted conjugates at 24 h and 48 h time points indicated that the dual-FAP conjugate was retained beyond 48 h, whereas the mono-FAP conjugate was substantially less detectable at both time points, approaching the detectability threshold by 48 h.

### INCORPORATION BY REFERENCE

All the patents, patent application publications, journal articles, books and other publications cited herein are hereby incorporated by reference.

### EQUIVALENTS

Those skilled in the art will recognize or be able to ascertain using no more than routine experimentation, many equivalents to the various embodiments of the disclosure described herein. Such equivalents are encompassed by the following claims.

1. A compound having the structure (Q-L^{Q})ₘ-Y-L^{X}-X, wherein
each Q-L^{Q} is an arm of the compound;
m is the number of (Q-L^{Q}) arms in the compound,
m is an integer 2, 3, 4, 5, or 6;
Q is a ligand that binds to fibroblast activation protein (FAP) on a target cell;
L is a spacer that (i) connects Q to Y and (ii) provides length for the arms of the compound to reach multiple adjacent FAPs on the target cell;
Y is a multipoint template to which the multiple arms of the compound connect;
L^{X} is a spacer that connects X to Y; and
X is an active agent.

2.-4. (canceled)

5. The compound of claim 1, wherein Q has the structure: 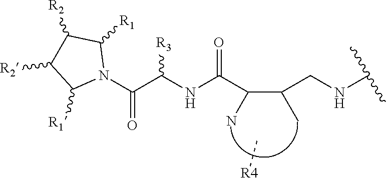
wherein
R₁, R₁′ are the same or different, and are independently selected from the group consisting of —H, alkyl, aryl, nitrile, —COOH, —B(OH)₂, SO₃H, and PO₃H;
R₂, R₂′ are the same or different, and are independently selected from the group consisting of H, halo, dihalo, alkyl, aryl, and heteroaryl;
R₃ is H, CH₃, alkyl, alkenyl, aryl, or heteroaryl; and
R₄ is H, alkyl, alkenyl, aryl, heteroaryl, halo, dihalo, dialkyl, or diaryl.

6.-10. (canceled)

11. The compound of claim 5, wherein the heterocycle 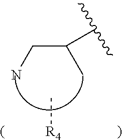
is selected from the group consisting of:

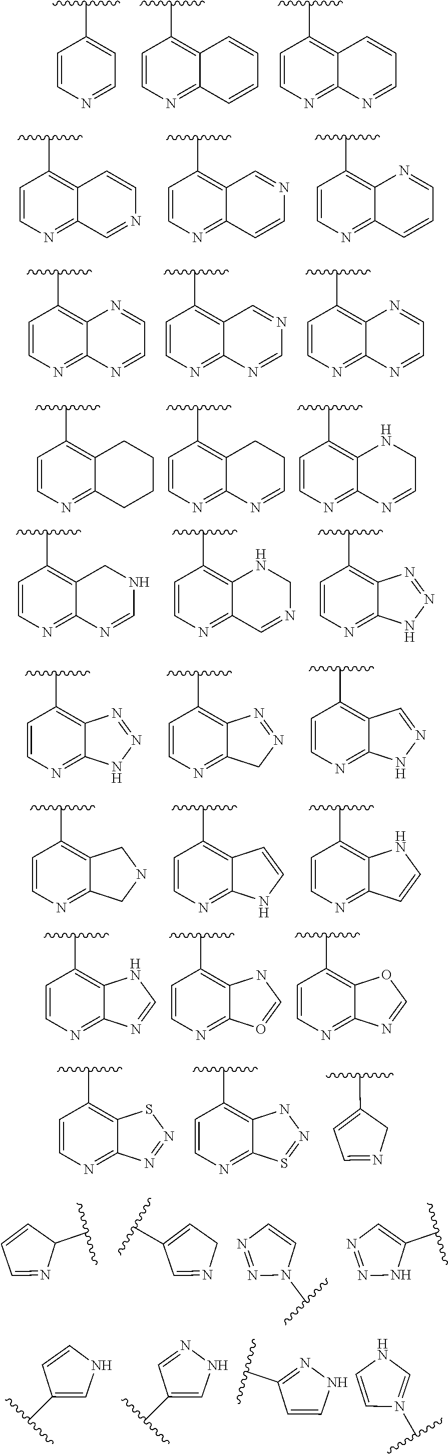

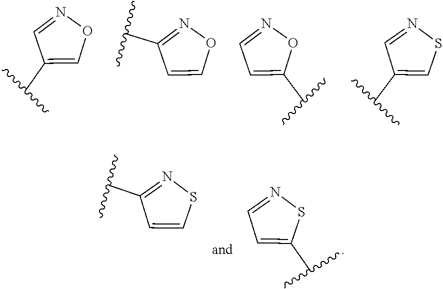

12. (canceled)

13. The compound of claim 1, wherein L^{Q} comprises an oligoethylene glycol, a polyethylene glycol, an alkyl chain, a peptidoglycan, an oligopeptide, or a polypeptide.

14.-17. (canceled)

18. The compound of claim 1, wherein L^{Q} is a spacer with a length between 15-200 angstroms.

19.-22. (canceled)

23. The compound of claim 1, wherein Y comprises one of the following structures: 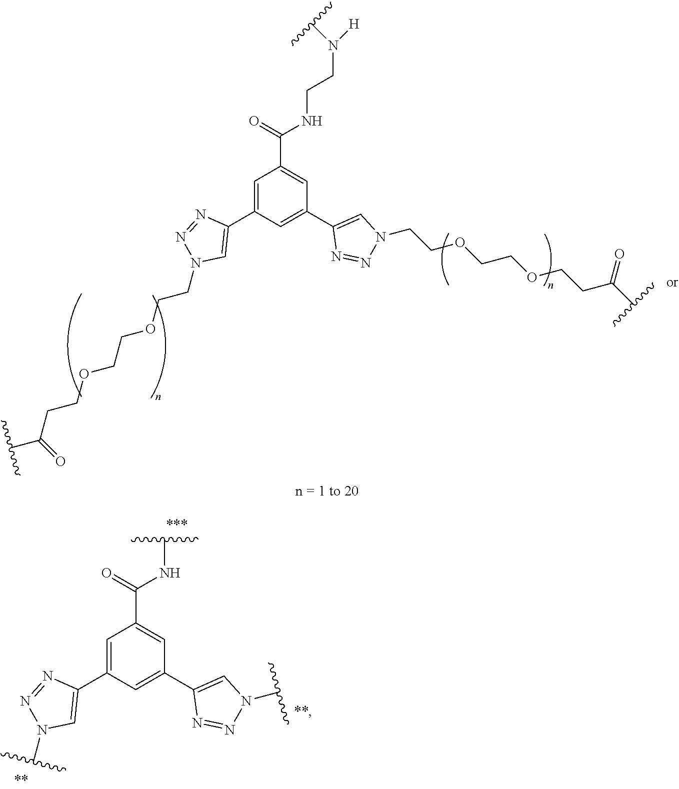
wherein ** indicates the point of attachment between Y and L^{Q}, and *** indicates the point of attachment between Y and L^{X}.

24. The compound of claim 1, wherein Y comprises the following structure: 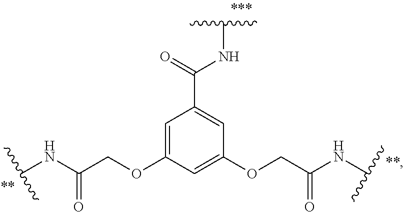 [SMILES: *NC(=O)COc1cc(OCC(=O)N*)cc(C(=O)N*)c1]
wherein ** indicates the point of attachment between Y and L9, and *** indicates the point of attachment between Y and L^{X}.

25. The compound of claim 1, wherein the compound comprises the following structure:

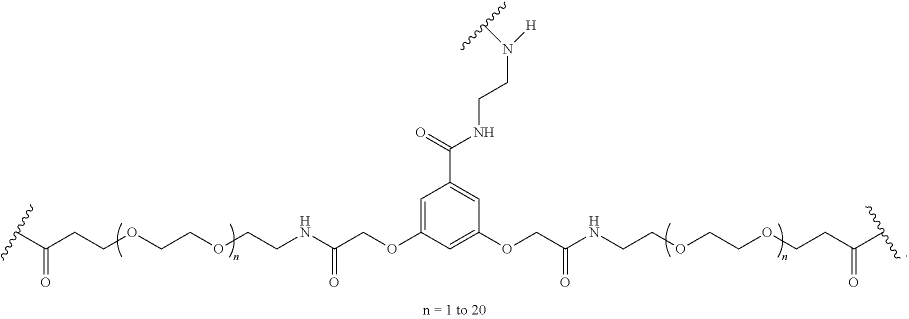

26.-34. (canceled)

35. The compound of claim 1 wherein L^{X} comprises one of the following structures between Y and X: 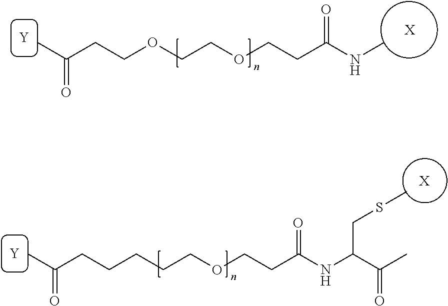
wherein n=1 to 32, and X and Y are points of attachment.

36. The compound of claim 1, wherein L^{X} comprises one of the following structures: 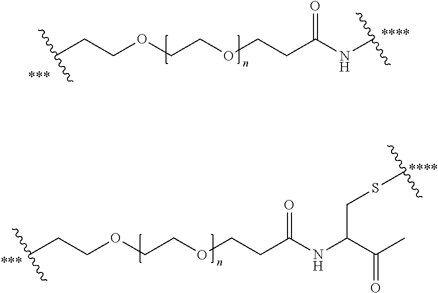
wherein n=1 to 32, *** indicates a point of attachment between Y and L^{X}, and **** indicates a point of attachment between X and L^{X}.

37.-38. (canceled)

39. The compound of claim 1, wherein L^{X} comprises one of the following structures, exclusive of Y and X: 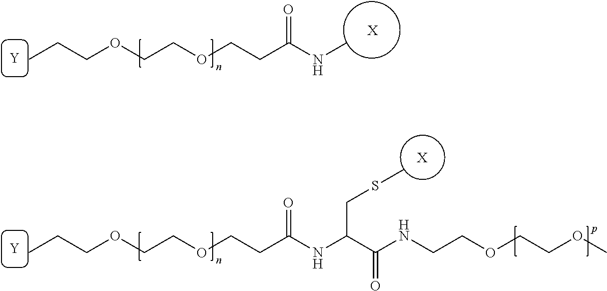
wherein n=1 to 32, p=1-32, and X and Y are points of attachment.

40. The compound of claim 1, wherein L^{X} comprises one of the following structures: 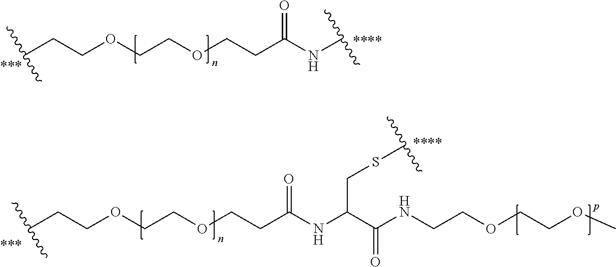
wherein n=1 to 32, p=1-32, *** indicates a point of attachment between Y and L^{X}, and **** indicates a point of attachment between X and L^{X}.

41.-42. (canceled)

43. The compound of claim 1, wherein L^{X} comprises one of the following structures exclusive of Y and X: 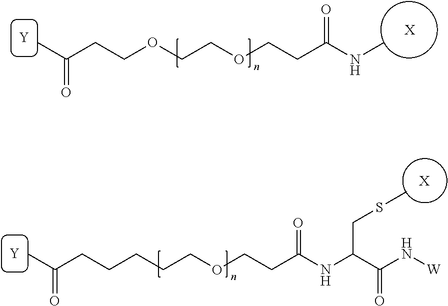
wherein W comprises a solubility enhancer, a PK/PD modulator, or a combination of two or more of either or both the foregoing, and wherein X and Y are points of attachment.

44. The compound of claim 1, wherein L^{X} comprises one of the following structures: 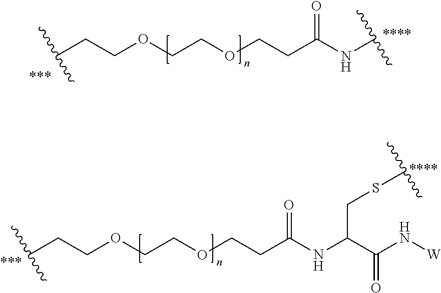
wherein W comprises a solubility enhancer, a PK/PD modulator, or a combination of two or more of either or both the foregoing, and wherein *** represents an attachment between Y and L^{X}, and **** represents an attachment between X and L^{X}.

45.-50. (canceled)

51. The compound of claim 1, wherein X comprises a chelating group with a structure selected from the group consisting of:

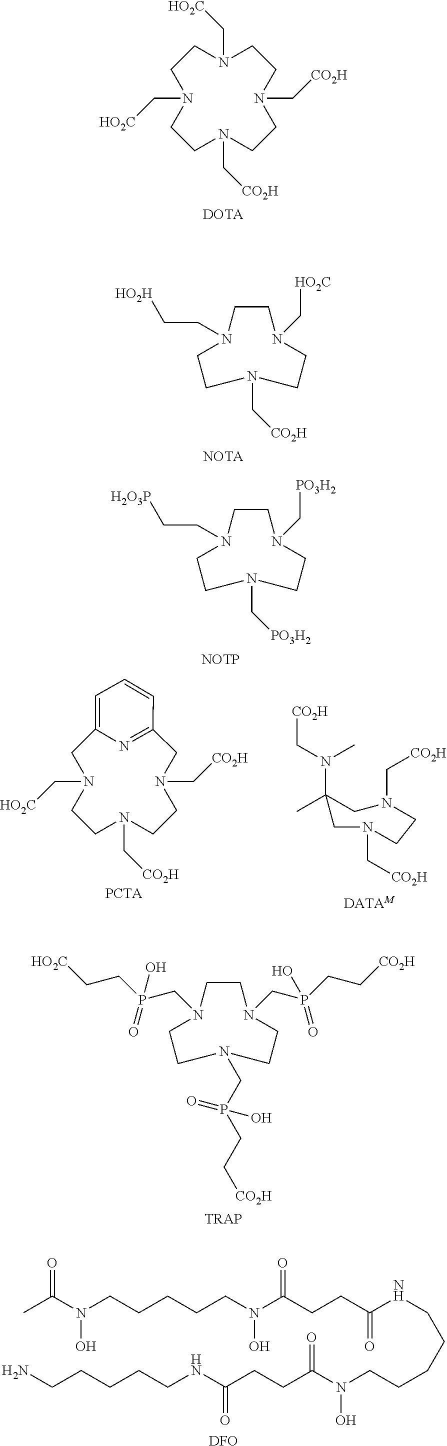

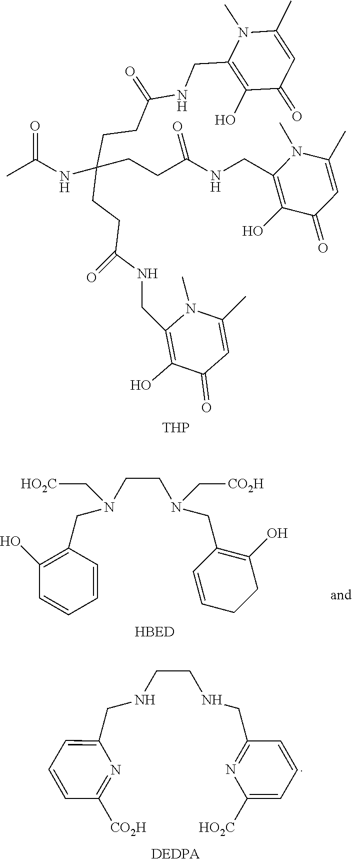 [SMILES: CC(=O)NC(CCC(=O)NCc1c(O)c(=O)cc(C)n1C)(CCC(=O)NCc1c(O)c(=O)cc(C)n1C)CCC(=O)NCc1c(O)c(=O)cc(C)n1C ; "THP" ; O=C(O)CN(CCN(CC(=O)O)Cc1ccccc1O)CC1=C(O)CCC=C1 ; O=C(O)c1cccc(CNCCNCc2cccc(C(=O)O)n2)n1]

52.-62. (canceled)

63. A compound that has the following structure:

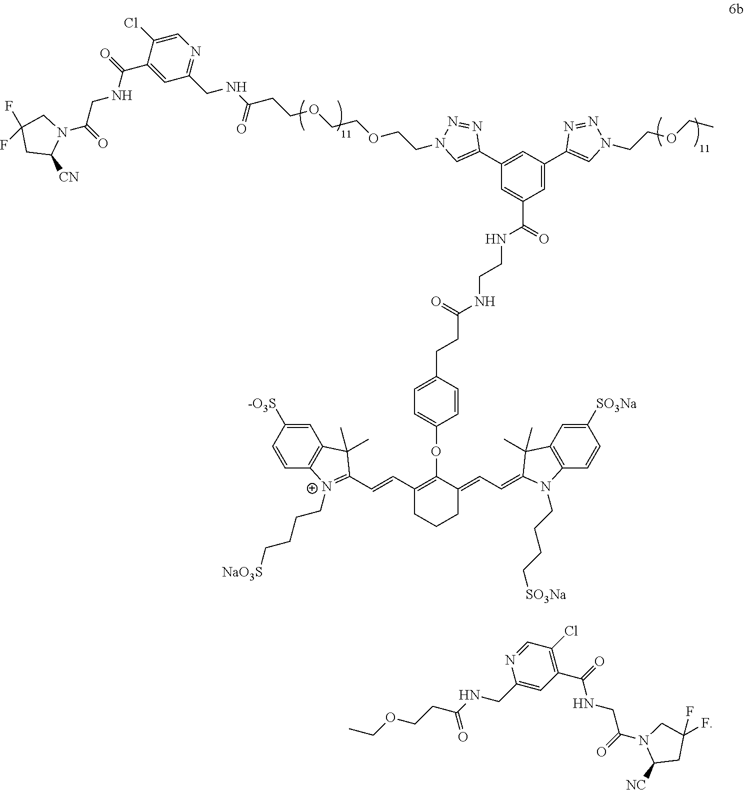

64. A compound that has the following structure:

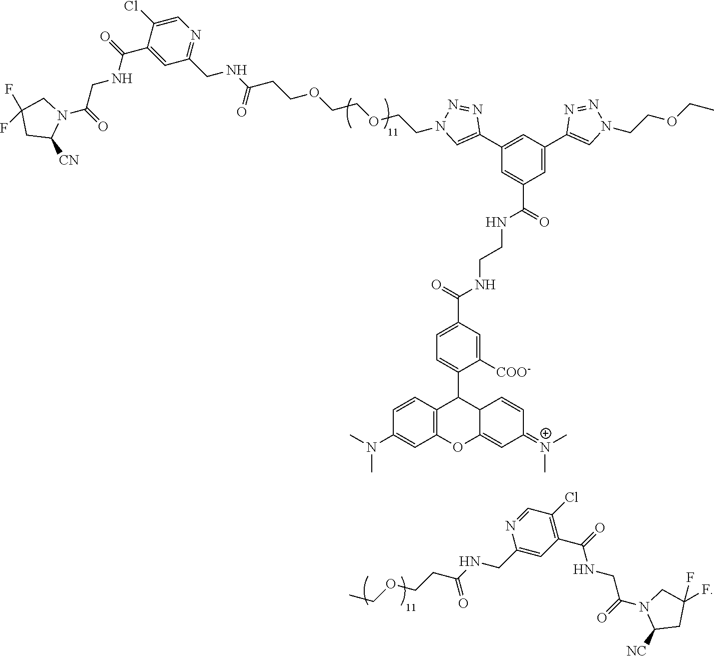

65. A compound that has the following structure:

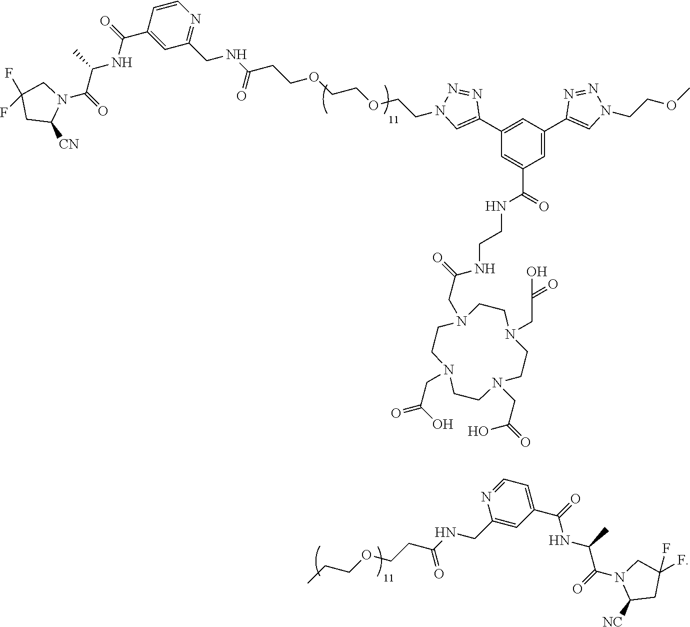

66. A compound that has the following structure:

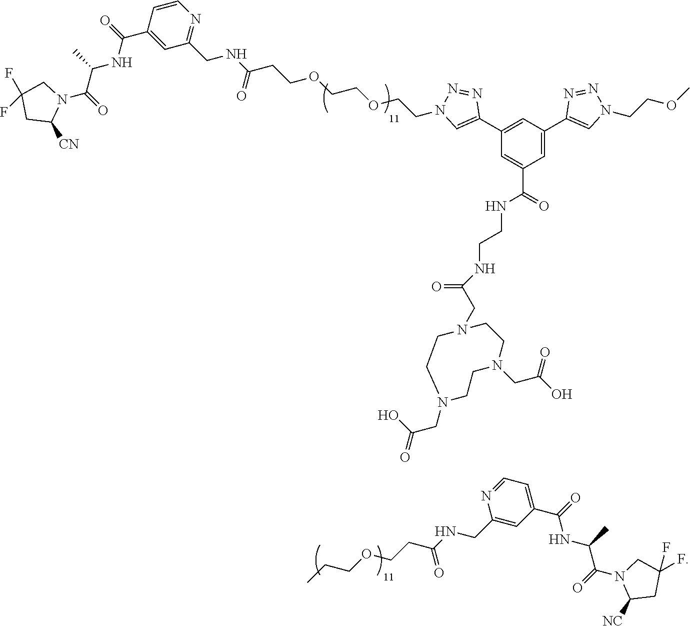

67. A compound that has the following structure:

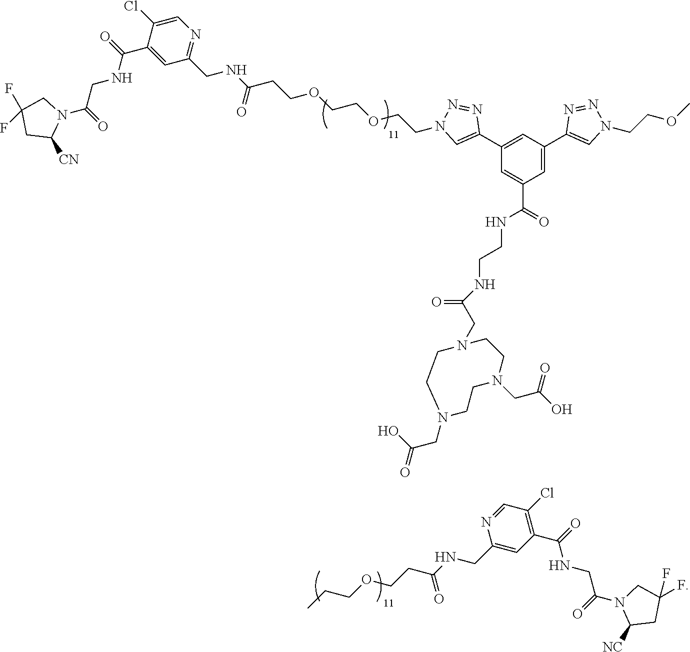

68. A pharmaceutical composition comprising a compound of claim 1 and a pharmaceutically acceptable carrier.

69. A method of delivering an active agent in proximity to a cancer-associated fibroblast (CAF) or a fibroblast activation protein (FAP)-expressing cell, comprising administering a compound of claim 1 to a cell expressing CAF or FAP, whereupon the compound is retained within the CAF or FAP-expressing cell for at least 24 hours.

70. A method of detecting the presence of a tumor or a fibrotic tissue in a subject, comprising (i) administering a compound of claim 1 to the subject, (ii) detecting the compound within the subject (e.g., optically or radiometrically), and (iii) identifying a tumor or a fibrotic tissue in the subject based on the localization of the compound, whereupon the presence of a tumor or a fibrotic tissue is detected in the subject.

71. A method of treating a tumor or a fibrotic tissue in a subject, comprising administering to the subject a therapeutically effective amount of a compound of claim 1, whereupon the subject is treated for a tumor or a fibrotic tissue.
